(19) United States
(12) Patent Application Publication
(10) Pub. No.: US 2019/0231761 A1
(43) Pub. Date: Aug. 1, 2019

(54) COMPOSITIONS AND METHODS FOR TARGETING FRUCTOSE ENZYMES AND TRANSPORTERS FOR THE TREATMENT OF CANCER

(71) Applicant: Duke University, Durham, NC (US)

(72) Inventor: Xiling Shen, Durham, NC (US)

(21) Appl. No.: 16/261,341

(22) Filed: Jan. 29, 2019

Related U.S. Application Data
(60) Provisional application No. 62/741,710, filed on Oct. 5, 2018.
(60) Provisional application No. 62/658,168, filed on Apr. 16, 2018.
(60) Provisional application No. 62/623,065, filed on Jan. 29, 2018.

(51) Int. Cl. A61K 31/44; C12N 15/113; C07K 16/40; A61K 35/00

(52) U.S. Cl. CPC A61K 31/44; C12N 15/1137; C07K 16/40; C12N 2310/141; C12N 2310/14; C12N 2310/122; A61K 35/00

(57) ABSTRACT

The disclosure relates to compositions and methods of treating cancer in a subject in need thereof, the method comprising administering to the subject an effective amount of a therapeutic agent capable of down-regulating and/or inhibiting a fructose enzyme or fructose transporter in a cell of the subject such that the cancer growth is suppressed.

### CROSS-REFERENCE TO RELATED APPLICATIONS

This application claims priority to U.S. Provisional Patent Application No. 62/623,065, filed Jan. 29, 2018, U.S. Provisional Patent Application No. 62/658,168, filed Apr. 16, 2018, and U.S. Provisional Patent Application No. 62/741,710, filed Oct. 5, 2018, the disclosure of each of which is hereby incorporated by cross-reference in its entirety.

### STATEMENT REGARDING FEDERALLY SPONSORED RESEARCH OR DEVELOPMENT

This application was made with United States government support under Federal Grant No. R21CA201963 awarded by the NIH-NCI. The United States government has certain rights in this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 5.58 kilobytes in size, and titled 19-037-US_SequenceListing_ST25.txt.

### BACKGROUND OF DISCLOSURE

### Field

The present disclosure provides compositions and methods for treating cancer in a subject in need thereof, the method comprising administering to the subject an effective amount of a therapeutic agent capable of down-regulating and/or inhibiting a fructose enzyme or a fructose transporter in a cell of the subject such that the cancer growth is suppressed.

### Technical Background

Primary tumors gradually accumulate genetic alterations and are influenced by their microenvironment until they acquire the ability to metastasize to distant organs (Gupta, G. P. et al., (2006) Cell, 127:679-695; Valastyan, S. et al., (2011) Cell, 147:275-292). Typical of this process, colorectal (CRC) progresses through an adenoma-to-carcinoma sequence that eventually leads to metastasis (Barker, N., et al., (2009) Nature, 457:608-611; Clevers, H., (2006) Cell, 127:469-480), preferentially (˜70% patients) to the liver (Rothbarth, J., et al., (2005) Ann. Oncol., 16 Supp. 2:ii144-149). At this phase, the disease becomes challenging to treat and eventually develops resistance to most forms of combination therapy, making CRC metastasis a leading cause of cancer-related deaths (Andre, T. et al., (2004) New England J. of Medicine, 350:2343-2351; Meyerhardt, J. A., (2005), New England J. of Medicine, 352:476-487). Patients with inoperable liver metastasis respond poorly to chemotherapeutic intervention and have a median survival of 6 to 9 months (Alberts, S. R., et al., (2005) J. of Clinical Oncology, 23:9243-9249). Liver lesions have also been shown to seed tertiary tumors in the lungs of patients (Nguyen, D. X., et al., (2009) Nat. Rev. Cancer, 9:274-284).

Cancer metastasis continues to account for the majority of cancer-related deaths and remains a clinical challenge. Current chemotherapy for advanced CRC does not target liver metastases specifically. This is partly based on observations that CRC metastases are not consistently associated with any specific genetic mutations (Jones, S., et al., (2008) Proc Natl Acad Sci USA, 105:4283-4288) and they generally resemble cells in the primary tumor. But it remains largely unclear how metastatic cancer cells may be influenced by the physiology of the organs they colonize. However, emerging evidence suggests that non-genetic alterations, such as epigenetic and metabolic reprogramming, may promote cancer metastasis, including CRC (Dupuy, F., et al., (2015) Cell Metab, 22:577-589; LeBleu, V. S, et al., (2014) Nature Cell Biology, 16:992-1003; Loo J. M., et al., (2015) Cell, 160:393-406; Piskounova, E. et al., (2015) Nature, 527:186-191; Ragusa, S., et al., (2014) Cell Rep, 8:1957-1973; Singovski, G., et al., (2016) J Mol Cell Biol, 8(2):157-173; Wu, Z., et al., (2015) Cell Stem Cell, 17:47-59). In particular, via GATA6, liver metastases upregulate ALDOB, an enzyme involved in fructose metabolism. Given that 70% of fructose is metabolized in the liver (Mayes, P. A., (1993) Nutrition, 58:754S-765S), targeting such mechanisms can enhance therapeutics against metastasis.

Thus, there is a need for treatments that target metastases. Described herein is a unique treatment for metastatic liver cancer using compositions and methods that target enzymes and proteins involved in fructose catalysis, transport, and metabolism.

### BRIEF SUMMARY OF DISCLOSURE

The present disclosure provides compositions and methods of treating metastatic cancer. One aspect of the disclosure provides a composition comprising a therapeutic agent for targeting a fructose enzyme or a fructose transporter in a cell, the composition being capable of inhibiting the function of a fructose enzyme or fructose transporter and/or down-regulating the gene expression of a fructose enzyme or fructose transporter in a cell. In some embodiments of the disclosure, the fructose enzyme or fructose transporter is selected from the group consisting of aldolase B (ALDOB), ketohexokinase (KHK), aldose reductase, sorbitol dehydrogenase, GLUT5, or GLUT2. In some embodiments of the disclosure, the therapeutic agent is an RNAi polynucleotide, a small molecule, or an antibody.

In some embodiments of the disclosure, the therapeutic agent is a small molecule inhibitor of KHK that is selected from the group consisting of, pyrimidinopyrimidine 1, indazole 2, pyrrolopyridine 7, pyrrolopyridine 9, pyrrolopyridine 10, pyridine 8, pyridine 11, pyridine 12, any combinations thereof, and any salts, esters, isomers, and derivatives thereof. In other embodiments of the disclosure, the therapeutic agent is a small molecule inhibitor of aldose reductase that is selected from the group consisting of alrestatin, epairestat, fidarestat, imirestat, lidoestat, minalrestat, ponalrestat, ranirestat, salfredin B₁₁, sorbinil, tolrestat, zenarestat, zopolrestat, any combinations thereof, and any salts, esters, isomers, and derivatives thereof. In other embodiments of the disclosure, the therapeutic agent is a small molecule inhibitor of sorbitol dehydrogenase that is selected from the group consisting of CP-470711 (SDI-711), WAY-135706, any combinations thereof, and any salts, esters, isomers, and derivatives thereof. In other embodiments of the disclosure, the therapeutic agent is a small molecule inhibitor of GLUT5 that is selected from the group consisting of N-[4-(methylsulfonyl)-2-nitrophenyl]-1,3-benzodioxol-5-amine, glyco-1,3-oxazolidin-2-thiones (OZT), glyco-1,3-oxazolidin-2-ones (OZO), any combinations thereof, and any salts, esters, isomers, and derivatives thereof. In other embodiments of the disclosure, the therapeutic agent is a small molecule inhibitor of GLUT2 that is selected from the group consisting of ertugliflozin, empagliflozin, canagliflozin, dapagliflozin, ipragliflozin, phloretin, myricetin, fisetin, quercetin, isoquercitrin, sappanin-type (SAP) homoisoflavonoids, any combinations thereof, and any salts, esters, isomers, and derivatives thereof.

In some embodiments of the disclosure, the therapeutic agent is an antibody against a fructose enzyme or fructose transporter. In some embodiments of the disclosure, the antibody is a neutralizing antibody against a fructose enzyme or fructose transporter. In other embodiments of the disclosure, the therapeutic agent is an anti-GLUT5 antibody selected from the group consisting of AGT-025, ab36057, ab41533, ab113931, ab87847, ab190555, or ab111299, OTI20E1. In other embodiments of the disclosure, the therapeutic agent is an anti-GLUT2 antibody selected from the group consisting of AGT-022, 600-401-GN3, LS-B15821, or LS-B4177.

In some embodiments of the disclosure, the therapeutic agent is an RNA interference (RNAi) polynucleotide that is capable of knocking down a fructose enzyme or a fructose transporter in a cell. In some embodiments of the disclosure, the RNAi polynucleotide is an shRNA. In some embodiments of the disclosure, the shRNA has a nucleotide sequence of any of SEQ ID NOS:1-2 and is capable of knocking down ALDOB in a cell. In other embodiments of the disclosure, the shRNA has a nucleotide sequence of any of SEQ ID NOS:3-7 and is capable of knocking down KHK in a cell. In other embodiments of the disclosure, the shRNA has a nucleotide sequence of any of SEQ ID NOS: 18-22 and is capable of knocking down aldose reductase in a cell. In yet other embodiments of the disclosure, the shRNA has a nucleotide sequence of any of SEQ ID NOS:23-27 and is capable of knocking down sorbitol dehydrogenase. In yet other embodiments of the disclosure, the shRNA has a nucleotide sequence of SEQ ID NOS: 8-12 and is capable of knocking down GLUT5 in a cell. In yet other embodiments of the disclosure, the shRNA has as sequence of SEQ ID NOS: 13-17 and is capable of knocking down GLUT2 in a cell.

Another aspect of the disclosure provides a method of treating cancer in a subject in need thereof, the method comprising administering to the subject an effective amount of a therapeutic agent capable of down-regulating and/or inhibiting a fructose enzyme or fructose transporter in a cell of the subject such that the cancer growth is suppressed. In some embodiments of the disclosure, the cancer is a metastatic cancer. In other embodiments of the disclosure, the cancer is a liver cancer. In other embodiments of the disclosure, the cancer is a metastatic liver cancer.

In some embodiments of the disclosure, the therapeutic agent is an RNAi polynucleotide, a small molecule, or an antibody.

In some embodiments of the disclosure, the RNAi polynucleotide is selected from the group consisting of small interfering RNA (siRNA), short hairpin RNA (shRNA), and microRNA (miRNAs) oligonucleotides.

In some embodiments of the disclosure, the fructose enzyme or fructose transporter is selected from aldolase B (ALDOB), ketohexokinase (KHK), aldose reductase, sorbitol dehydrogenase, GLUT5, or GLUT2.

In some embodiments of the disclosure, the small molecule is an inhibitor of aldolase B (ALDOB), ketohexokinase (KHK), aldose reductase, sorbitol dehydrogenase, GLUT5, or GLUT2. In other embodiments of the disclosure, the small molecule blocks de novo fructose synthesis in a cell of the subject.

In some embodiments of the disclosure, the small molecule is selected from the group consisting of pyrimidinopyrimidine 1, indazole 2, pyrrolopyridine 7, pyrrolopyridine 9, pyrrolopyridine 10, pyridine 8, pyridine 11, pyridine 12, any combinations thereof, and any salts, esters, isomers, and derivatives thereof. In other embodiments of the disclosure, the small molecule is pyridine 12.

In some embodiments of the disclosure, the small molecule is selected from the group consisting of alrestatin, epairestat, fidarestat, imirestat, lidoestat, minalrestat, ponalrestat, ranirestat, salfredin B₁₁, sorbinil, tolrestat, zenarestat, zopolrestat, any combinations thereof, and any salts, esters, isomers, and derivatives thereof.

In some embodiments of the disclosure, the small molecule is CP-470711 (SDI-711) and any salts, esters, isomers, and derivatives thereof.

In some embodiments of the disclosure, the method of treating cancer in a subject in need thereof further comprises restricting the dietary intake of fructose in the subject. In some embodiments of the disclosure, the subject has no dietary intake of fructose.

Another aspect of the disclosure provides a method of treating cancer in a subject in need thereof, the method comprising administering to the subject an effective amount of a therapeutic agent capable of blocking de novo fructose synthesis in the subject such that the cancer growth is suppressed.

In some embodiments of the disclosure, the therapeutic agent is a small molecule inhibitor of or antibody against aldose reductase or sorbitol dehydrogenase.

Another aspect of the disclosure provides a method of suppressing cancer growth in a subject in need thereof, the method comprising down-regulating and/or inhibiting a fructose enzyme or fructose transporter in a cell of the subject. In some embodiments, the fructose enzyme or fructose transporter is selected from the group consisting of aldolase B (ALDOB), aldose reductase, sorbitol dehydrogenase, ketohexokinase (KHK), GLUT5, or GLUT2.

In some embodiments, the cell is contacted with a fructose enzyme or fructose transporter inhibitor selected from the group consisting of pyrimidinopyrimidine 1, indazole 2, pyrrolopyridine 7, pyrrolopyridine 9, pyrrolopyridine 10, pyridine 8, pyridine 11, pyridine 12, alrestatin, epairestat, fidarestat, imirestat, lidoestat, minalrestat, ponalrestat, ranirestat, salfredin B₁₁, sorbinil, tolrestat, zenarestat, zopolrestat, CP-470711 (SDI-711), AGT-025, ab36057, ab41533, ab113931, ab87847, ab190555, ab111299, OTI20E1, AGT-022, 600-401-GN3, LS-B15821, or LS-B4177 and any salts, esters, isomers, and derivatives thereof.

Additional features and advantages are described herein, and will be apparent from the Drawings, Detailed Description, and the Claims.

### BRIEF DESCRIPTION OF THE DRAWINGS

The foregoing aspects and other features of the disclosure are explained in the following description, taken in connection with the accompanying drawings, wherein:

FIG. 1A-1E illustrates a comparison of metabolic states of primary CRC and liver metastasis. FIG. 1A is a volcano plot of differential metabolic gene expression between paired normal colon and primary CRC samples from 30 CRC liver metastasis patients selected from 4 public GEO datasets. Each circle represents a gene. Significantly up-regulated genes have a p value <0.05 and fold change >2, while significantly down-regulated genes have a p value <0.05 and fold change <0.5. FIG. 1B is a volcano plot of differential metabolic gene expression between paired primary CRC and liver metastases samples from 30 CRC liver metastasis patients selected from 4 public GEO datasets. Each circle represents a gene. Significantly up-regulated genes have a p value <0.05 and fold change >2, while significantly down-regulated genes have a p value <0.05 and fold change <0.5. FIG. 1C is a graph of Gene Set Enrichment Analysis (GSEA) of up-regulated metabolic pathways in liver metastases based on comparison of the paired samples. FIG. 1D is a Venn Diagram of differential analysis. Top: the significantly up-regulated (p value<0.05, fold change>2) genes in Liver Mets and Lung Mets comparing to primary CRCs. Bottom: the significantly down-regulated (p value<0.05, fold change<0.5) genes in Liver Mets and Lung Mets comparing to primary CRCs. FIG. 1E is a graph of Gene Set Enrichment Analysis. Each panel shows the pathway analysis of the up-regulated (right-facing bars) or down-regulated genes (left-facing bars) in liver metastases only, lung metastases only, and commonly altered genes respectively.

FIG. 2 is a representation of the up-regulated glycolysis/gluconeogenesis (top) and pentose phosphate (bottom) pathways and MS peak intensity of their corresponding intermediate metabolites of primary colon tumor (left bar in each panel) and liver metastases (right bar in each panel). *, p<0.05; ***, p<0.001. p-value was calculated based on linear model.

FIG. 3A-3D illustrates metabolomic analysis of the in vivo model. FIG. 1A are MA plots of Liver metastases vs primary tumors (left), lung metastases vs primary tumors (middle), and liver metastases vs lung metastases (right) based on differential analysis. Each dot represents a compound. Darker dot are differentially regulated (p value<0.05) compounds: up-regulation (logFC>1); down-regulation (logFC<−1). The radius of the dot is associated with p value-larger dots correspond to smaller p values. FIG. 3B is a graph showing Metabolite Set Enrichment Analysis (MSEA) comparing liver and lung metastases using MetaboloAnalyst. The metabolites sets shown were filtered based on p value <0.05 and FDR <0.1. Hits refer to the number of compounds overlapping with the compounds list in the pathways. FIG. 3C is a matrix analysis of metabolite clustering on the metabolomics of primary colon tumors, lung metastases and liver metastases. The similarity matrix is based on Euclidean distance analysis to evaluate the metabolomics difference between samples using Morpheus. FIG. 3D are FACS plots isolating mCherry+HCT116 cells from primary cecum tumors and liver metastases.

FIG. 4 is a graph showing integrated pathway analysis of transcriptomic and metabolomics data. The significantly enriched (p value<0.05, fold change>1) genes from RNA-seq and significantly enriched (p value<0.05, fold change>1) metabolites from metabolomics comparing liver metastases samples to primary tumor samples were integrated by combining hypergeometric test for enrichment analysis and degree centrality in topology analysis based on gene-metabolite pathways using Metabolyst. The identified enrichment pathway list is compared with the clinical enriched pathway (FIG. 1C) and the consistently enriched pathways are shown. x axis: p values from hypergeometric test, y axis: hits refers to the number of upregulated metabolites/genes overlapping with the ones in the metabolic pathway. The bar refers to the topology analysis that calculates the importance of the genes and metabolites on its position within a metabolic pathway based on degree centrality.

FIG. 5 is a diagram of ALDOB in fructose metabolism.

FIG. 6 are paired box plots comparing expression levels of ALDOB, ALDOA, KHK, HK1, HK2 and GLUT5 between matched samples of normal colon, primary CRC, and liver metastasis from 30 patients in 4 GEO datasets (Table 2). Dots refer to different samples, and lines connect the paired samples. Different shapes refer to different datasets. ***, p<0.001. p-values were calculated based on paired linear model using Limma.

FIG. 7 shows FACS analysis of ALDOB, KHK and HK levels in HCT116, CRC119 and CRC57 CRC cells.

FIG. 8 are paired box plots showing the expression levels of ALDOB, ALDOA, KHK, HK1, HK2 and GLUT5 in DNA microarray data analysis on 39 primary colon carcinoma and 74 liver metastasis samples from stage IV CRC patients. p-values were calculated based on linear model using Limma. ***, p<0.001.

FIG. 9 is a schematic and representative IVIS luciferase in vivo images of the orthotopic/metastatic cecum injection mouse model.

FIG. 10 are paired box plots showing the expression levels of ALDOB, ALDOA, KHK, HK1, HK2 and GLUT5 in DNA microarray data analysis on 39 primary colon carcinoma, 74 liver metastasis and 8 lung metastasis samples from stage IV CRC patients. p-values were calculated based on linear model using Limma. ***, p<0.001.

FIG. 11 are graphs showing Gene Set Enrichment Analysis (GSEA) of up-regulated metabolic pathways in liver metastases based on comparison of the paired samples.

FIG. 12A is a Western blot showing ALDOB expression increased in liver metastases compared to primary cecum tumors derived from cecum-injected HCT116 cells. FIG. 12B is a Western blot showing ALDOB expression increased in liver metastases compared to primary cecum tumors derived from cecum-injected CRC119 cells. FIG. 12C is a Western blot showing ALDOB expression increased in liver metastases compared to primary cecum tumors derived from cecum-injected and CRC57 cells.

FIG. 13 are Western blots of ALDOB levels in CRC cells isolated from primary cecum tumor (C) and lung metastases (L).

FIG. 14 is a schematic and representative IVIS luciferase in vivo images of simultaneous cecum and intrahepatic injection mouse model.

FIG. 15A is a Western blot showing higher ALDOB expression in liver tumors than in cecum tumors from HCT116 cells. FIG. 15B is a Western blot showing higher ALDOB expression in liver tumors than in cecum tumors from CRC119 cells. FIG. 15C is a Western blot showing higher ALDOB expression in liver tumors than in cecum tumors from CRC57 cells.

FIG. 16A is a schematic of the trans-well migration assay. FIG. 16B is a Western blot showing ALDOB expression in migrated and nonmigrated HCT116 CRC cells. FIG. 16C is a Western blot showing ALDOB expression in migrated and nonmigrated CRC119 CRC cells.

FIG. 16D is a Western blot showing ALDOB expression in migrated and nonmigrated CRC57 CRC cells.

FIG. 17 are Western blots of ALDOB levels in CRC cells isolated from primary cecum tumor (C) and liver metastases (L) after culturing in vitro for 3 days.

FIG. 18 is a schematic of GATA6 binding motif in ALDOB promoter.

FIG. 19 is a graph of ChIP-qPCR showing enrichment of GATA6 binding to the ALDOB promoter in CRC cells isolated from liver metastases compared to those from primary cecum tumors. Error bars denote SD of triplicates.

FIG. 20 is a Western blot showing up-regulation of ALDOB in response to fructose under hypoxia is dependent on GATA6.

FIG. 21A-21D illustrates metabolism analysis of CRC liver metastases. FIG. 21A are images of Periodic Acid Schiff (PAS) staining of normal colon, colon tumor, normal liver and liver metastases harvested from HCT116 cells tumor-bearing mice. FIG. 21B are images of Oil Red O (ORO) staining of normal colon, colon tumor, normal liver and liver metastases harvested from HCT116 cells tumor-bearing mice. FIG. 21C are images of PAS staining coupled with amylase digestion to identify glycogen deposits in the colon. Hematoxylin and Eosin staining of normal and tumor tissues harvested from tumor-bearing mice. Top: PAS staining; bottom: AS staining coupled with amylase digestion. FIG. 21D are images of PAS staining coupled with amylase digestion to identify glycogen deposits in the liver. Hematoxylin and Eosin staining of normal and tumor tissues harvested from tumor-bearing mice. Top: PAS staining; bottom: AS staining coupled with amylase digestion.

FIG. 22 are graphs of a seahorse assay measuring ECAR and OCR in HCT116 cells derived from liver metastases at baseline and following injection of 11 mM Fructose. Error bars denote SD of triplicates.

FIG. 23 is a Western blot showing ALDOB knockdown efficiency by two shRNAs (shALDOB1 and shALDOB2) in HCT116, CRC119, CRC57 and CT26 cells.

FIG. 24A-24B illustrate that ALDOB regulates fructose metabolism. FIG. 24A is a graph showing WST-1 cell proliferation assay of CRC cells with control or anti-ALDOB shRNA vectors cultured in glucose containing media with dialyzed FBS under hypoxia. Error bars denote SD of triplicates. FIG. 24B is a graph showing WST-1 cell proliferation assay of CRC cells with control or anti-ALDOB shRNA vectors cultured in fructose containing media with dialyzed FBS under hypoxia. Error bars denote SD of triplicates.

FIG. 25 is a tracing analysis using ¹³C labeled fructose by LC-MS. ¹³C labeled carbon was analyzed after cells were incubated in ¹³C labeled fructose containing medium for 24 hours. Three cell lines were measured in wild-type (WT) condition and ectopic ALDOB expression (OE). The bar diagrams show the enrichment percent, and error bars denote SD of triplicates. The schematic diagrams show the corresponding isotopomer transition from ¹³C labeled fructose, and the red circles represents the number of detected 13C labeled carbons in the intermediate metabolites.

FIG. 26 is a tracing analysis using ¹³C labeled fructose by LC-MS. ¹³C-labeled carbon was analyzed by GC-MS after cells were incubated in media containing ¹³C-labeled fructose and dialyzed FBS for 9 hours. WT and OE indicate ALDOB levels in wild type and over expression. Error bars denote SD of triplicates.

FIG. 27 is tracing analysis using ¹³C labeled carbon of other sugar monomers. Error bars denote SD of triplicates.

FIG. 28A-28C illustrates that silencing of ALDOB suppresses CRC liver metastasis. FIG. 28A shows a trans-well migration assay showing ALDOB knockdown does not affect HCT116 cell migration. Error bars denote SD of triplicates. FIG. 28B shows a trans-well migration assay showing ALDOB knockdown does not affect CRC119 cell migration. Error bars denote SD of triplicates. FIG. 28C shows a trans-well migration assay showing ALDOB knockdown does not affect CRC57 cell migration.

FIGS. 29A-29E illustrates CRC liver metastasis in mice with cecum injection of HCT116, CRC119, and CRC57 cells carrying dual luciferase/fluorescent reporter constructs. FIG. 29A is a schematic of the cecum injection model. FIG. 29B are representative IVIS luciferase in vivo images of mice with cecum injection of HCT116, CRC119 and CRC57 cells carrying dual luciferase/fluorescent reporter constructs. FIG. 29C show bright field and fluorescent images of livers, and quantification of liver metastasis in mice with cecum injection of HCT116 cells show ALDOB knockdown suppressed liver metastasis. FIG. 29D show bright field and fluorescent images of livers, and quantification of liver metastasis in mice with cecum injection of CRC119 cells show ALDOB knockdown suppressed liver metastasis. FIG. 29E show bright field and fluorescent images of livers, and quantification of liver metastasis in mice with cecum injection of CRC57 cells show ALDOB knockdown suppressed liver metastasis.

FIG. 30A shows a schematic of the intrahepatic injection model. FIG. 30B are IVIS luciferase in vivo images showing CRC growth in liver with intrahepatic injection of HCT116, CRC119 and CRC57 cells carrying dual luciferase/fluorescence reporter constructs show ALDOB knockdown suppressed CRC growth in the liver. FIG. 30C are bright-field and fluorescent images of livers with intrahepatic injection of HCT116, CRC119 and CRC57 cells carrying dual luciferase/fluorescence reporter constructs show ALDOB knockdown suppressed CRC growth in the liver.

FIG. 31 are images and graphs of Ki-67 staining showing ALDOB knockdown suppressed CRC cells proliferation in the liver. Error bars denote SEM of 5 mice per group. *** p<0.001. p-values were calculated based on one-way ANOVA.

FIG. 32A are images and a graph showing CRC lung metastasis with cecum injection of HCT116 cells with ALDOB knockdown, or high or low fructose diets. FIG. 32B are images and a graph showing CRC lung metastasis with cecum injection of CRC119 cells, with ALDOB knockdown, or high or low fructose diets. FIG. 32C are images and a graph showing CRC lung metastasis with cecum injection of CRC57 cells with ALDOB knockdown, or high or low fructose diets.

FIG. 33A are images and a graph showing CRC lung metastasis with tail vein injection of HCT116 cells with ALDOB knockdown, or high or low fructose diets. Error bars denote SEM of 5 mice per group. FIG. 33B are images and a graph showing CRC lung metastasis with tail vein injection of CRC119 cells with ALDOB knockdown, or high or low fructose diets. Error bars denote SEM of 5 mice per group.

FIG. 34A are IVIS luciferase in vivo images showing CRC liver metastasis in BALB/c mice with cecum injection of CT26 cells. FIG. 34B is a graph showing that knockdown of ALDOB suppressed liver metastasis in immunocompetent BALB/c mice.

FIG. 35 is a graph showing Ki-67 quantification of staining showing knockdown of ALDOB suppressed CT26 cell proliferation in the liver.

FIG. 36A are representative IVIS luciferase in vivo images of mice with CRC cell HCT116, CRC119 and CRC57 injected in cecum and fed with a regular diet, a fructose-high diet, a fructose-restricted diet, or a fructose restricted diet+ALDOB knockdown. FIG. 36B, FIG. 36C, and FIG. 36D show bright field and fluorescent images of liver tissue from the mice in (FIG. 36A) with CRC cell HCT116, CRC119, and CRC57, respectively. Liver metastasis was quantified using the Image J software. Error bars denote SEM of 5 mice per group. p-values were calculated based on oneway ANOVA.

FIG. 37 are representative IVS images showing cecum injection of CT26 cells showing fructose-high diet promoted liver metastasis, while fructose-restricted diet with ALDOB knockdown suppressed liver metastasis in immunocompetent BALB/c mice.

FIG. 38 is a graph showing cecum injection of CT26 cells showing fructose-high diet promoted liver metastasis, while fructose-restricted diet with ALDOB knockdown suppressed liver metastasis in immunocompetent BALB/c mice. Error bars denote SEM of 5 mice per group. p-values were calculated based on one-way ANOVA. **, p<0.01; ***, p<0.001.

FIG. 39 are graphs showing survival curves of mice intrahepatically injected with CRC cells and fed with a regular diet, a fructose-high diet, a fructose-restricted diet, or a fructose restricted diet+ALDOB knockdown. Error bars denote s.d. of 5 mice per group. p value was calculated in comparison with normal diet group on the base of log-rank test. **, p<0.01; *** p<0.001.

FIG. 40 are graphs showing survival curves of mice in cecum injection mice model with CRC cells and fed with a regular diet, a fructose-high diet, a fructose-restricted diet, or a fructose-restricted diet+ALDOB knockdown. p value was calculated in comparison with normal diet group on the base of logrank test. *, p<0.05, **, p<0.01; ***, p<0.001.

FIG. 41A is representative IVIS luciferase in vivo images of mice with intravenous injection of liver-derived HCT116 cells with or without ALDOB knockdown. FIG. 41B are bright field and fluorescent (mCherry) images of livers of mice with intravenous injection of liver-derived HCT116 cells with or without ALDOB knockdown. FIG. 41C is a graph showing quantification of liver metastasis of mice with intravenous injection of liver-derived HCT116 cells with or without ALDOB knockdown, and show knockdown of ALDOB suppressed CRC liver lesions. FIG. 41D are representative IVIS luciferase in vivo images of mice with intravenous injection of liver-derived HCT116 with fructose diet. FIG. 41E are bright field and fluorescent (mCherry) images of livers of mice with intravenous injection of liver-derived HCT116 with fructose diet. FIG. 41F is a graph of quantification of liver metastasis of mice with intravenous injection of liver-derived HCT116 with fructose diet that show mice fed with fructose-restricted diet suppressed liver lesions. FIG. 41G are representative images of mice injected with liver metastasis derived mcherry labeled HCT116 and treated with normal saline, 5-Fluorouracil (5FU, 100 mg/kg), or Oxaliplatin (OXA, 6 mg/kg). FIG. 41H are fluorescent imaging of liver tissue from mice in FIG. 41G. FIG. 41I is a graph showing quantification of liver lesions. FIG. 41J is a graph showing the survival curve analysis of treated and untreated tumor-bearing mice from FIG. 41G. Error bars denote SEM of 3 mice per group. **, p<0.01; ***, p<0.001. p-value was calculated based on one-way ANOVA.

FIG. 42A-42B illustrates KHK knockdown suppresses liver metastasis. FIG. 42A is a Western blot showing KHK knockdown efficiency. FIG. 42B are representative IVIS luciferase in vivo images, bright field and fluorescent images of livers, and quantification of liver metastasis. Error bars denote SEM of 5 mice per group. p-values were calculated based on one-way ANOVA. ***, p<0.001.

FIG. 43A-43B illustrates GLUT5 knockdown suppresses liver metastasis. FIG. 43A is a Western blot showing GLUT5 knockdown efficiency. FIG. 43B are representative IVIS luciferase in vivo images of liver metastasis taken on Day 11, Day 14, and Day 17 following CRC transplant.

### DETAILED DESCRIPTION

For the purposes of promoting an understanding of the principles of the present disclosure, reference will now be made to specific embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

It will be further understood that a number of aspects and embodiments are disclosed. Each of these has individual benefit and each can also be used in conjunction with one or more, or in some cases all, of the other disclosed aspects and embodiments, whether specifically delineated or not. Accordingly, for the sake of clarity, this description will refrain from repeating every possible combination of the individual aspects and embodiments in an unnecessary fashion. Nevertheless, the specification and claims should be read with the understanding that such combinations are implicitly disclosed, and are entirely within the scope of the disclosure and the claims, unless otherwise specified.

Articles “a” and “an” are used herein to refer to one or to more than one (i.e. at least one) of the grammatical object of the article. By way of example, “an element” means at least one element and can include more than one element.

As used herein, the term “and/or” includes any and all combinations of one or more of the associated listed items. It will be further understood that the use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “about” in association with a numerical value means that the numerical value can vary plus or minus by 5% or less of the numerical value.

The inventor has discovered that metastatic colorectal cancer (CRC) cells reprogram their metabolism when they metastasize to the liver. As described herein, meta-analyses of extensive clinical datasets as well as integrated transcriptomics/metabolomics analysis of in vivo metastasis models systematically characterized metabolic alterations in CRC liver metastasis compared to primary tumor. Particularly, CRC cells up-regulate ALDOB to metabolize fructose, which is especially abundant in the liver, to fuel major pathways of central carbon metabolism (glycolysis/gluconeogenesis, PPP, and pyruvate entry into TCA) to promote tumor cell proliferation. The inventor has discovered that targeting ALDOB and other enzymes and transporters associated with fructose, and/or dietary restriction of fructose dramatically suppresses liver metastasis and outperforms frontline chemotherapy drugs. Furthermore, the inventor has discovered that the unique fructose-rich and hypoxic liver environment contributes to its popularity for cancer metastasis, and liver metastasis of other cancer types. Accordingly, the invention comprises compositions and methods for down-regulating and/or inhibiting fructose enzymes involved in fructose metabolism in the liver, or other upstream catalytic events involving the absorption, formation, or transport of fructose, to treat and suppress liver metastases.

Cancer and Liver Metastases

Cancer is generally considered a group of diseases involving abnormal, uncontrolled cell growth with the potential to spread, or metastasize, to other parts of the body. The term “cell” as used herein refers to the basic structural, functional, and biological unit of a living organism. A cell can be a cancer cell or a non-cancer cell. The term “cancer cell” as used herein refers to a cell that divides relentlessly, forming solid tumors or flooding the blood with abnormal cells, and that is able to spread from one part of the body to another. The term “non-cancer cell” as used herein refers to a cell that does not have the characteristics of a cancer cell (e.g., abnormal growth and spreading to other areas of the body). Non-cancer cells tend to stop growing when enough cells are present, respond to other cell signals to stop growth, and repair themselves or die when they are unhealthy. A non-cancer cell can be, for example, a normal liver cell.

The therapeutic agents and methods of the present disclosure can be used to treat any cancer, and any metastases thereof, including, but not limited to, carcinoma, lymphoma, blastoma, sarcoma, and leukemia. More particular examples of such cancers include breast cancer, prostate cancer, colorectal cancer (CRC), squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. As used herein, a “primary” tumor or cancer is a tumor growing at the anatomical site where the tumor progression began (e.g., primary liver cancer originates in the liver). In some embodiments, the cancer comprises liver cancer. In certain embodiments, the cancer comprises metastatic liver cancer.

A liver metastasis is a cancerous tumor that has spread to the liver from a cancer that started in another place in the body. The cancer cells found in a metastatic liver cancer cell are cells from the part of the body where the cancer originated (e.g., breast cancer cells, colon cancer cells, or lung cancer cells). Primary cancers that can spread to the liver include cancers of the breast, colon, rectum, kidney, esophagus, lung, skin, ovaries, uterus, pancreas, and stomach. Liver metastasis is also referred to as secondary liver cancer, liver metastases, metastases to the liver, and stage IV or advanced cancer. Thus, as used herein, “metastatic liver cancer” or “liver metastases” refers to cancerous cells that are found in the liver but originated outside of the liver.

Human liver metastases, among other cancers, can be studied in mouse models according the methods described herein and those known in the art. In particular, human cancer cell lines to CRC can be manipulated in vitro (e.g., undergo knockdown) and then implanted into livers of mice (e.g., NOD/SCID mice) using the CRC metastatic model. Human cancer cell lines can be implanted into the mouse, for example, via a cecum injection model or intrahepatic injection model. Human cancer cell lines that can be studied in a mouse model include, but are not limited to, the CRC cell line HCT116, patient derived xenograft human CRC cell line CRC119 and CRC57. Mouse cancer cell lines that can be used to study liver metastases include the BALB/c mouse colon cancer cell line CT26.

Fructose in the Liver

As described herein, liver metastases can be treated by down-regulating or silencing the gene expression of proteins and enzymes involved in absorbing, catalyzing, transporting, and metabolizing fructose.

Fructose can be found in foods either as a monosaccharide (free fructose) or as a unit of a disaccharide (sucrose). Free fructose is absorbed directly by the intestine. When fructose is consumed in the form of sucrose, it is broken down and then absorbed as free fructose. As sucrose comes into contact with the membrane of the small intestine, the enzyme sucrase catalyzes the cleavage of sucrose to yield one glucose unit and one fructose unit, which are then each absorbed.

Fructose absorption occurs on the mucosal membrane via facilitated transport involving fructose transporters. As used herein, the term “fructose transporter” refers to a trans membrane protein that moves fructose from one cellular environment to another. Fructose transporters include, but are not limited to, glucose transporter 5 (GLUT5), glucose transporter 2 (GLUT2), glucose transporter 3 (GLUT3), and glucose transporter 4 (GLUT4). Fructose transporters can be expressed on the surface of a cancer cell (e.g., a CRC cell) or on the surface of a non-cancer cell.

Fructose can also be formed from the sorbitol-aldose reductase pathway, or the polyol pathway. The sorbitol-aldose reductase pathway is a two-step process that converts glucose to fructose. First, aldose reductase reduces glucose to sorbitol. Second, sorbitol is oxidized by sorbitol dehydrogenase to fructose.

Among the altered metabolic pathways, fructose metabolism is unique in the context of the liver, because more than 70% of fructose is metabolized in the liver (Mayes, 1993). Fructose is therefore an abundant nutrient in the liver microenvironment and constitutes a significant carbon source for bioenergetics. Fructose contributes to de novo glucose production through its entrance at the triose kinase-mediated step. Fructose is first metabolized by ketohexokinase (KHK) or hexokinase (HK). Subsequently, fructose-1-phosphate (F1P) is converted into glyceraldehyde and dihydroxyacetone phosphate (DHAP) in a reversible reaction catalyzed by ALDOB. Glyceraldehyde is then phosphorylated by the triose kinase and the resulting glyceraldehyde-3-phosphate (GAP) can either serve as a glycolytic substrate or condense with DHAP into F1,6BP through the action of ALDOB to enter the gluconeogenic pathway (Feinman, R. D., et. al., (2013) Nutrition & Metabolism, 10:45-45). As one of the three aldolase isoforms (A, B, and C), ALDOB shows comparable activity toward F1P and F1,6BP and participates in both glycolysis and gluconeogenesis pathways (Penhoet, E., et al., (1966) Proc of Nat Acad Sci USA, 56:1275-1282). The products of ALDOB-mediated reaction could contribute to glucose, glycogen, lactate, and lipid synthesis, all essential for sustaining highly proliferative cells. Fructose metabolism could also cause glycogen and lipid deposits (Stanhope, K. L., et al., (2009) J. of Clinical Investigation, 119:1322-1334).

The impact of fructose on CRC liver metastasis may not be limited to cancer cells alone. Fructose-enriched diets can induce liver damage, obesity, glucose intolerance, hepatomegaly, and nonalcoholic fatty liver disease in animal models. Fructose can enhance the progression of non-alcoholic fatty liver disease and clinical liver fibrosis, which are risk factors for liver cancer (Abdelmalek, M. F. et al., (2012) Hepatology, 56:952-960; Abdelmalek, M. F., et al., (2010) Hepatology, 51:1961-1971). Hence, diets high in fructose may disrupt normal liver homeostasis to create a more conducive environment for tumor growth in addition to providing fuel for CRC cell metabolism.

The enzymes that are involved in the absorption, formation (e.g., via the sorbitol-aldose reductase pathway or hydrolysis of sucrose to fructose), or metabolism of fructose (e.g., enzymes in the KHK metabolic pathway, enzymes in the ALDOB metabolic pathway) are referred to as “fructose enzymes.” Fructose enzymes include, but are not limited to, sucrase, fructokinase, which is also referred to as ketohexokinase (KHK), hexokinase (HK), aldolase A, aldolase B (ALDOB), aldolase C, aldose reductase, and sorbitol dehydrogenase.

Therapeutic Agents for Targeting Fructose Enzymes and Transporters

The present disclosure provides, in part, therapeutic agents for targeting fructose enzymes and fructose transporters for the treatment of cancer (e.g., liver metastases) in a subject. One aspect of the present disclosure provides a composition comprising a therapeutic agent for targeting a fructose enzyme or a fructose transporter in a cell, the composition being capable of inhibiting the function of a fructose enzyme or fructose transporter and/or down-regulating the gene expression of a fructose enzyme or fructose transporter in a cell.

As used herein, the term “therapeutic agent” refers to a compound that is capable of inhibiting the function of a fructose enzyme or fructose transporter protein or down-regulating or inhibiting the gene expression of a fructose enzyme or a fructose transporter protein in a cell (e.g., a cancer cell or non-cancer cell). A therapeutic agent can be, for example, a small molecule, an RNA interference (RNAi) polynucleotide, an oligonucleotide, a peptide, or an antibody.

In some embodiments, the therapeutic agent is a small molecule or antibody that targets and binds with high affinity (e.g., an apparent Kd value in the micromolar, nanomolar, or picomolar range) and specificity to a fructose enzyme or fructose transporter and inhibits the function of the fructose enzyme or fructose transporter. Thus, the term “inhibitor” as used herein refers to a therapeutic agent that inhibits the function of a fructose enzyme or fructose transporter.

As used herein, the terms “inhibits the function” or “the function is inhibited” and the like in reference to a fructose enzyme means that the ability of the fructose enzyme to catalyze a reaction is lost or reduced relative to the activity of the enzyme in the absence of an inhibitor or below the level observed in the presence of a control. The terms “inhibits the function” or “the function is inhibited” and the like in reference to a fructose transporter means that the ability of the fructose transporter to transport a fructose unit is lost or reduced relative to the activity of the transporter in the absence of an inhibitor or below the level observed in the presence of a control.

In some embodiments, more than one therapeutic agent can be used in combination to target multiple different fructose enzymes and/or multiple different fructose transporters at the same time.

In some embodiments, the therapeutic agent targets and inhibits the function of ketohexokinase. KHK loss of function mutations in humans are asymptopmatic, making it a safe therapeutic target. Examples of small molecules inhibitors of KHK are provided in Huard, K. et al. (2017) J. Med. Chem., 60, 7835-7849, the entirety of which is hereby incorporated by reference. Examples of KHK small molecule inhibitors include, but are not limited to, pyrimidinopyrimidine 1, indazole 2, pyrrolopyridine 7, pyrrolopyridine 9, pyrrolopyridine 10, pyridine 8, pyridine 11, pyridine 12, any combinations thereof, and any salts, esters, isomers, and derivatives thereof, the structures of which are shown below.

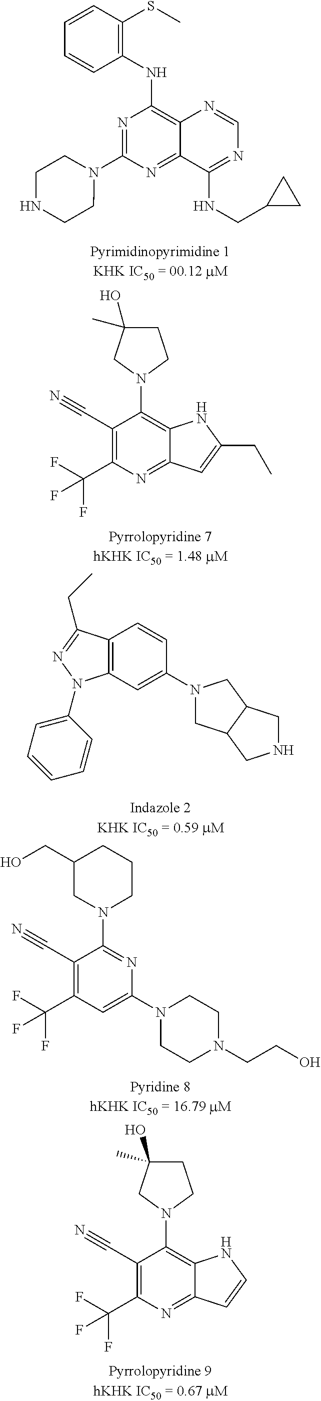 [SMILES: CSc1ccccc1Nc1nc(N2CCNCC2)nc2c(NCC3CC3)ncnc12 ; CCc1cc2nc(C(F)(F)F)c(C#N)c(N3CCC(C)(O)C3)c2[nH]1 ; CCc1nn(-c2ccccc2)c2cc(N3CC4CNCC4C3)ccc12 ; N#Cc1c(C(F)(F)F)cc(N2CCN(CCO)CC2)nc1N1CCCC(CO)C1 ; C[C@]1(O)CCN(c2c(C#N)c(C(F)(F)F)nc3cc[nH]c23)C1]

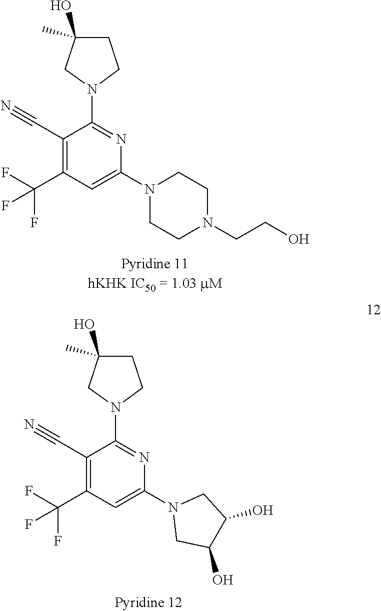 [SMILES: C[C@]1(O)CCN(c2nc(N3CCN(CCO)CC3)cc(C(F)(F)F)c2C#N)C1 ; C[C@]1(O)CCN(c2nc(N3C[C@H](O)[C@@H](O)C3)cc(C(F)(F)F)c2C#N)C1]

In certain embodiments, the small molecule comprises pyridine 12 and any salts, esters, isomers, and derivatives thereof.

In some embodiments, the therapeutic agent targets and inhibits the function of aldose reductase. Examples of aldose reductase small molecule inhibitors include, but are not limited to, alrestatin, epairestat, fidarestat, imirestat, lidoestat, minalrestat, ponalrestat, ranirestat, salfredin B₁₁, sorbinil, tolrestat, zenarestat, zopolrestat, any combinations thereof, and any salts, esters, isomers, and derivatives thereof, the structures of which are shown below:

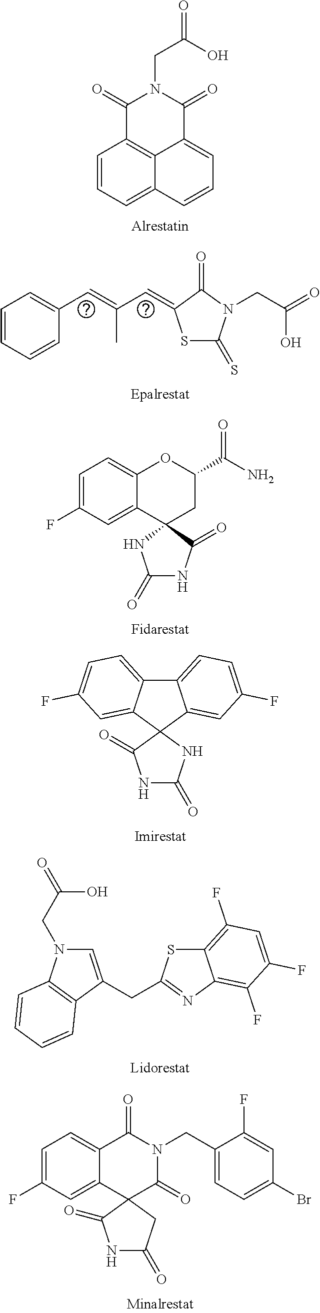 [SMILES: O=C(O)CN1C(=O)c2cccc3cccc(c23)C1=O ; CC(/C=C1\SC(=S)N(CC(=O)O)C1=O)=C\c1ccccc1 ; NC(=O)[C@@H]1C[C@]2(NC(=O)NC2=O)c2cc(F)ccc2O1 ; O=C1NC(=O)C2(N1)c1cc(F)ccc1-c1ccc(F)cc12 ; O=C(O)Cn1cc(Cc2nc3c(F)c(F)cc(F)c3s2)c2ccccc21 ; O=C1CC2(C(=O)N1)C(=O)N(Cc1ccc(Br)cc1F)C(=O)c1ccc(F)cc12]

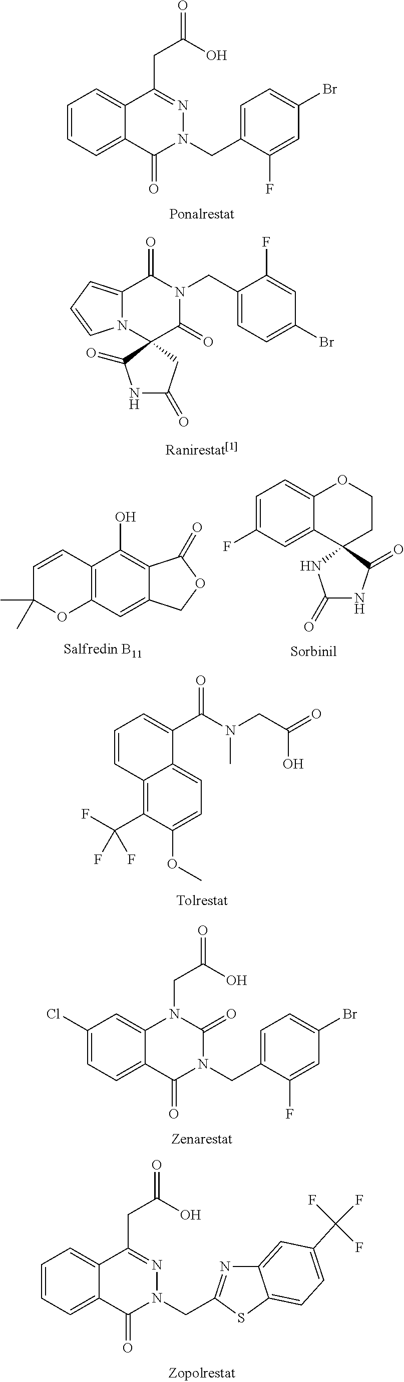

In another embodiment, the therapeutic agent targets and inhibits the function of sorbitol dehydrogenase. Small molecule inhibitors of sorbitol dehydrogenase include, but are not limited to, CP-470711 (SDI-711), WAY-135706, any combinations thereof, and any salts, esters, isomers, and derivatives thereof.

In another embodiment, the therapeutic agent targets and inhibits the function of aldolase (e.g., aldolase A, B, or C). Small molecule inhibitors of aldolase include, but are not limited to, phosphorylated α-dicarbonyl compounds (e.g., phosphoric acid mono-(2,3-dioxo-butyl) ester; Charbot, N. et al. (2008) J. of Enzyme Inhibition and Med. Chem., 23(1):21-27), and Compounds 1 and 2 as described in Daher, M. et al., (2010) ACS Med. Chem. Lett., 1:101-104, and any salts, esters, isomers, and derivatives thereof.

In another embodiment, the therapeutic agent targets and inhibits the function of a fructose transporter. In some embodiments, the therapeutic agent targets and inhibits the function of GLUT5. Small molecule inhibitors of GLUT5 include, but are not limited to, N-[4-(methylsulfonyl)-2-nitrophenyl]-1,3-benzodioxol-5-amine (MSNBA) (WO2016201214), glyco-1,3-oxazolidin-2-thiones (OZT) (e.g., D-arabinose-OZT, L-arabinose-OZT, D-xylose-OZT, D-ribose-OZT, D-fructose-Bn-OZT, D-fructose-allyl-OZT, D-fructose-OZT, L-sorbose-allyl-OZT, L-sorbose-Bn-OZT, and L-sorbose-OZT), and glyco-1,3-oxazolidin-2-ones (OZO) (e.g., D-fructose-Bn-OZO, D-fructose-OZO, L-sorbose-Bn-OZO, and L-sorbose-OZO) (Girniene, J., et al., (2003)) Carbohydrate Research, 338:711-719, and any salts, esters, isomers, and derivatives thereof. In other embodiments, the therapeutic agent targets and inhibits the function of GLUT2. Small molecule inhibitors of GLUT2 include, but are not limited to ertugliflozin, empagliflozin, canagliflozin, dapagliflozin, ipragliflozin, phloretin, myricetin, fisetin, quercetin, isoquercitrin, sappanin-type (SAP) homoisoflavonoids, and any salts, esters, isomers, and derivatives thereof.

In other embodiments, the therapeutic agent is an antibody that targets and binds with specific activity against a fructose enzyme or fructose transporter such that the function of the fructose enzyme or fructose transporter is inhibited. Antibodies against a fructose enzyme include, but are not limited to anti-human ALDOB antibody (e.g., PA5-30218, 1:2000, Pierce), anti-Hexokinase antibody (e.g., C35C4, 1:1000, Cell Signaling), and anti-ketohexokinase antibody (e.g., 4B8, 1:2000, Abcam). In some embodiments, the antibody is a neutralizing antibody against a fructose enzyme or fructose transporter. The term “neutralizing antibody” as used herein refers to an antibody that binds to and inhibits the function of the antigen (e.g., a fructose enzyme or fructose transporter). Neutralizing antibodies against a fructose transporter include, but are not limited to anti-GLUT5 antibodies (e.g., AGT-025, ab36057, ab41533, ab113931, ab87847, ab190555, ab111299, OTI20E1) and anti-GLUT2 antibodies (e.g., AGT-022, 600-401-GN3, LS-B15821, LS-B4177). In some embodiments, more than one antibody against a fructose enzyme and/or fructose transporter can be used to target and inhibit multiple different fructose enzymes and/or multiple different fructose transporters at the same time.

In other embodiments, the therapeutic agent is an RNA interference (RNAi) polynucleotide that targets and knocks down the gene of a fructose enzyme or fructose transporter. In some embodiments, more than one RNAi polynucleotide can be used to target and knockdown multiple different fructose enzymes and/or multiple different fructose transporters at the same time.

The term “RNA interference (RNAi) polynucleotide” as used herein refers to a molecule capable of inhibiting, down-regulating, or reducing expression or translation of a target gene by neutralizing target mRNA. Examples of an RNA interference (RNAi) polynucleotide include, but are not limited to, double stranded RNA (dsRNA), antisense oligonucleotides (ASO), small interfering RNA (siRNA), short hairpin RNA (shRNA), microRNA (miRNAs) oligonucleotides, and aptamers, and the like.

The terms “down-regulate” or “knockdown” are used herein to refer to reducing the level of RNA transcribed from the target gene or the level of a polypeptide, protein or protein subunit translated from the RNA, below the level that is observed in the absence of the blocking therapeutic agent of the disclosure or below the level observed in the presence of a control inactive therapeutic agent (e.g., a polynucleotide with a scrambled sequence or with inactivating mismatches). RNAi-mediated degradation of the target mRNA can be detected by measuring levels of the target mRNA or protein in the cells of a subject, using standard techniques for isolating and quantifying mRNA or protein. In some embodiments, knocking down ALDOB, KHK, aldose reductase, sorbitol dehydrogenase, GLUT2, or GLUT5 in a cancer cell (e.g., a CRC cell) and/or a non-cancer cell can significantly suppress metastatic growth in the liver.

The term “up-regulate” as used herein refers to increasing the level of RNA transcribed from the target gene, or the level of a polypeptide, protein or protein subunit translated from the RNA, or the level of metabolites produced by a cell, above the level that is observed in the absence of a therapeutic agent, a control inactive therapeutic agent, or in the absence of an abnormal cellular state (e.g., liver metastases).

The terms “polynucleotide” and “oligonucleotide” refer to sequences with conventional nucleotide bases, sugar residues and internucleotide phosphate linkages, but also to those that contain modifications of any or all of these moieties. The term “nucleotide” as used herein includes those moieties that contain not only the natively found purine and pyrimidine bases adenine (A), guanine (G), thymine (T), cytosine (C), and uracil (U), but also modified or analogous forms thereof. Polynucleotides include RNA and DNA sequences of more than one nucleotide in a single chain. Modified RNA or modified DNA, as used herein, refers to a molecule in which one or more of the components of the nucleic acid, namely sugars, bases, and phosphate moieties, are different from that which occurs in nature.

Design, synthesis, and purification of RNA interference (RNAi) polynucleotides can be performed by established methods known in the art.

As used herein, the term “target mRNA” means mRNA of a fructose enzyme or fructose transporter of the subject (e.g., a human, mouse, rat, etc.).

Expression of RNAi (e.g., shRNA) in cells can be achieved by delivery plasmids or through vectors (e.g., bacterial or viral vectors). Delivery of plasmids to cells through transfection to obtain RNAi expression can be accomplished using commercially available reagents in vitro. RNAi expression in cells can also be achieved by using a bacterial vector. For example, recombinant Escherichia coli containing a plasmid with RNAi that is fed to mice can knock-down target gene expression in the intestinal epithelium. A variety of viral vectors can be used to obtain RNAi expression in cells including adenoviruses, lentiviruses, and adeno-associated viruses (AAVs).

The term “double-stranded RNA (dsRNA)” is RNA with two complementary strands, similar to the DNA found in all cells. dsRNA forms the genetic material of some viruses (double-stranded RNA viruses). Double-stranded RNA such as viral RNA or siRNA can trigger RNA interference in eukaryotes, as well as interferon response in vertebrates.

The term “antisense oligonucleotides (ASO)” as used herein refers to the use of a nucleotide sequence, complementary by virtue of Watson-Crick base pair hybridization, to a specific mRNA to inhibit its expression and then induce a blockade in the transfer of genetic information from DNA to protein. The ASO molecule can be complementary to a portion of the coding or noncoding region of an RNA molecule, e.g., a pre-mRNA or mRNA. An ASO molecule can be, for example, about 10 to 25 nucleotides in length. An ASO molecule can be constructed using chemical synthesis and/or enzymatic ligation reactions using procedures known in the art. Alternatively, the ASO molecule can be transcribed biologically using an expression vector into which a nucleic acid has been subcloned in an antisense orientation (i.e., RNA transcribed from the inserted nucleic acid will be of an antisense orientation to a target nucleic acid of interest).

The term “small interfering RNA (siRNA),” also known as short interfering RNA or silencing RNA, is used herein to refer to a class of double-stranded RNA molecules, approximately 10-50 base pairs in length, but preferably 19-25 base pairs in length that interferes with the expression of specific target genes with complementary nucleotide sequences by degrading mRNA after transcription, preventing translation. An siRNA can have a nucleotide sequence identical (perfectly complementary) or substantially identical (partially complementary) to a portion of the coding sequence in an expressed target gene or RNA within the cell. An siRNA may have short 3′ overhangs. An siRNA may be composed of two annealed polynucleotides or a single polynucleotide that forms a hairpin structure. An siRNA molecule of the disclosure comprises a sense region and an antisense region. In one embodiment, the siRNA of the disclosure is assembled from two oligonucleotide fragments wherein one fragment comprises the nucleotide sequence of the antisense strand of the siRNA molecule and a second fragment comprises the nucleotide sequence of the sense region of the siRNA molecule. In certain embodiments, the siRNA are chemically modified. In another embodiment, the sense strand is connected to the antisense strand via a linker molecule, such as a polynucleotide linker or a non-nucleotide linker. An siRNA can be constructed using chemical synthesis and/or enzymatic ligation reactions using procedures known in the art. Alternatively, the siRNA nucleic acid can be transcribed biologically using an expression vector into which a nucleic acid has been subcloned in an antisense orientation (i.e., RNA transcribed from the inserted nucleic acid will be of an antisense orientation to a target nucleic acid of interest).

The term “short hairpin RNA (shRNA)” as used herein refers to an artificial RNA molecule with a tight hairpin turn that can be used to silence target gene expression via RNAi. shRNA is an advantageous mediator of RNAi because it has a relatively low rate of degradation and turnover. Due to the ability of shRNA to provide specific, long-lasting, gene silencing, shRNA is a promising candidate for gene therapy applications, such as for cancer treatment. An shRNA can be constructed using chemical synthesis and/or enzymatic ligation reactions using procedures known in the art. Alternatively, the shRNA nucleic acid molecule can be transcribed biologically using an expression vector (plasmids or viral or bacterial vectors) into which a nucleic acid has been subcloned in an antisense orientation (i.e., RNA transcribed from the inserted nucleic acid will be of an antisense orientation to a target nucleic acid of interest). An shRNA of the present disclosure can contain about 45 to 65 nucleotides (e.g., about 45, 46, 47, 48, 49, 50, 51, 52, 53, 54, 55, 56, 57, 58, 59, 60, 61, 62, 63, 64, 65 nucleotides).

In some embodiments, an shRNA molecule can be used to knockdown a fructose enzyme (e.g., ALDOB, KHK, aldose reductase, or sorbitol dehydrogenase) or a fructose transporter (e.g., GLUT5 or GLUT2) in a cancer cell (e.g., a CRC cell) and/or a non-cancer cell. shRNA molecules that can be used to knockdown ALDOB in a cell include, but are not limited to, the nucleotide sequences of SEQ ID NOS: 1-2. shRNA molecules that can be used to knockdown KHK in a cell include, but are not limited to, the nucleotide sequences of SEQ ID NOS:3-7. shRNA molecules that can be used to knockdown aldose reductase in a cell include, but are not limited to, the nucleotide sequences of SEQ ID NOS:18-22. shRNA molecules that can be used to knockdown sorbitol dehydrogenase in a cell include, but are not limited to, the nucleotide sequences of SEQ ID NOS:23-27. shRNA molecules that can be used to knockdown GLUT5 in a cell include, but are not limited to, the nucleotides of SEQ ID NOS: 8-12. shRNA molecules that can be used to knockdown GLUT2 in a cell include, but are not limited to, the nucleotide sequences of SEQ ID NOS: 13-17.

The nucleotide sequences for shRNA molecules that can be used to knockdown the gene expression of a fructose enzyme and/or a fructose transporter are shown in Table 1.

TABLE 1
shRNA knockdown sequences against fructose enzymes and
transporters

| Gene Name | shRNA ID | Colon ID | Species | Sequence |
|---|---|---|---|---|
| ALDOB | SHCLNG- | TRCN000 | Human | CCGGCTCAGAAATTGCCCAGAGCATCTCGAGAT |
| | NM_000035 | 0052511 | | GCTCTGGGCAATTTCTGAGTTTTTG (SEQ ID NO: 1) |
| ALDOB | SHCLNG- | TRCN000 | Human | CCGGGTGGGAATCAAGTTAGACCAACTCGAGTT |
| | NM_000035 | 0052510 | | GGTCTAACTTGATTCCCACTTTTTG (SEQ ID NO: 2) |
| Fructokinase | SHCLNG- | TRCN000 | Human | CCGGGCTACAGACTTTGAGAAGGTTCTCGAGAA |
| (KHK) | NM_000221 | 0037725 | | CCTTCTCAAAGTCTGTAGCTTTTTG (SEQ ID NO: 3) |
| Fructokinase | SHCLNG- | TRCN000 | Human | CCGGCCTAAGGAGGACTCGGAGATACTCGAGTA |
| (KHK) | NM_000221 | 0037727 | | TCTCCGAGTCCTCCTTAGGTTTTTG (SEQ ID NO: 4) |
| Fructokinase | SHCLNG- | TRCN000 | Human | CCGGGCAGCGGATAGACGCACACAACTCGAGTT |
| (KHK) | NM_000221 | 0037728 | | GTGTGCGTCTATCCGCTGCTTTTTG (SEQ ID NO: 5) |
| Fructokinase | SHCLNG- | TRCN000 | Human | CCGGGACTCGGAGATAAGGTGTTTGCTCGAGCA |
| (KHK) | NM_000221 | 0199677 | | AACACCTTATCTCCGAGTCTTTTTTG (SEQ ID |
| | | | | NO: 6) |
| Fructokinase | SHCLNG- | TRCN000 | Human | CCGGGCCAGATGTGTCTGCTACAGACTCGAGTCT |
| (KHK) | NM_000221 | 0199463 | | GTAGCAGACACATCTGGCTTTTTTG (SEQ ID |
| | | | | NO: 7) |
| GLUT5 | SHCLNG- | TRCN000 | Human | CCGGGCACTGCTCATGCAACAATTTCTCGAGAAA |
| | NM_003039 | 0043003 | | TTGTTGCATGAGCAGTGCTTTTTG (SEQ ID NO: 8) |
| GLUT5 | SHCLNG- | TRCN000 | Human | CCGGCGCCACATCATTTGAGCTTATCTCGAGATA |
| | NM_003039 | 0043004 | | AGCTCAAATGATGTGGCGTTTTTG (SEQ ID NO: 9) |
| GLUT5 | SHCLNG- | TRCN000 | Human | CCGGCCCGTACAGCTTCATTGTCTTCTCGAGAAG |
| | NM_003039 | 0043005 | | ACAATGAAGCTGTACGGGTTTTTG (SEQ ID |
| | | | | NO: 10) |
| GLUT5 | SHCLNG- | TRCN000 | Human | CCGGCCTTGCTGTTCAACAACATATCTCGAGATA |
| | NM_003039 | 0043006 | | TGTTGTTGAACAGCAAGGTTTTTG (SEQ ID NO: 11) |
| GLUT5 | SHCLNG- | TRCN000 | Human | CCGGCAAGACGTTCATAGAGATCAACTCGAGTT |
| | NM_003039 | 0043007 | | GATCTCTATGAACGTCTTGTTTTTG (SEQ ID |
| | | | | NO: 12) |
| GLUT2 | SHCLNG- | TRCN000 | Human | CCGGGCCCACAATCTCATACTCAATCTCGAGATT |
| | NM_000340 | 0043598 | | GAGTATGAGATTGTGGGCTTTTTG (SEQ ID NO: 13) |
| GLUT2 | SHCLNG- | TRCN000 | Human | CCGGGCAAACATTCTGTCATTAGTTCTCGAGAAC |
| | NM_000340 | 0043600 | | TAATGACAGAATGTTTGCTTTTTG (SEQ ID NO: 14) |
| GLUT2 | SHCLNG- | TRCN000 | Human | CCGGGCACCTCAACAGGTAATAATACTCGAGTAT |
| | NM_000340 | 0043602 | | TATTACCTGTTGAGGTGCTTTTTG (SEQ ID NO: 15) |
| GLUT2 | SHCLNG- | TRCN000 | Human | CCGGCGACGTTCTCTCTTTCTAATTCTCGAGAATT |
| | NM_000340 | 0043601 | | AGAAAGAGAGAACGTCGTTTTTG (SEQ ID NO: 16) |
| GLUT2 | SHCLNG- | TRCN000 | Human | CCGGGCTGAATAAGTTCTCTTGGATCTCGAGATC |
| | NM_000340 | 0043599 | | CAAGAGAACTTATTCAGCTTTTTG (SEQ ID NO: 17) |
| Aldose | SHCLNG- | TRCN000 | Human | CCGGTGTGCCCATGTGTACCAGAATCTCGAGATT |
| reductase | NM_001628 | 0046410 | | CTGGTACACATGGGCACATTTTTG (SEQ ID NO: 18) |
| Aldose | SHCLNG- | TRCN000 | Human | CCGGCCATTGGATGAGTCGGGCAATCTCGAGATT |
| reductase | NM_001628 | 0046412 | | GCCCGACTCATCCAATGGTTTTTG (SEQ ID NO: 19) |
| Aldose | SHCLNG- | TRCN000 | Human | CCGGGTTCCCAGTGACACCAACATTCTCGAGAAT |
| reductase | NM_001628 | 0046409 | | GTTGGTGTCACTGGGAACTTTTTG (SEQ ID NO: 20) |
| Aldose | SHCLNG- | TRCN000 | Human | CCGGCCATTGGATGAGTCGGGCAATCTCGAGATT |
| reductase | NM_001628 | 0288738 | | GCCCGACTCATCCAATGGTTTTTG (SEQ ID NO: 21) |
| Aldose | SHCLNG- | TRCN000 | Human | CCGGTGCTGAGAACTTTAAGGTCTTCTCGAGAAG |
| reductase | NM_001628 | 0046411 | | ACCTTAAAGTTCTCAGCATTTTTG (SEQ ID NO: 22) |
| Sorbitol | SHCLNG- | TRCN000 | Human | CCGGGCGCCTGGAGAACTATCCTATCTCGAGATA |
| dehydrogenase | NM_003104 | 0028100 | | GGATAGTTCTCCAGGCGCTTTTT (SEQ ID NO: 23) |
| Sorbitol | SHCLNG- | TRCN000 | Human | CCGGGAGAACTATCCTATCCCTGAACTCGAGTTC |
| dehydrogenase | NM_003104 | 0028069 | | AGGGATAGGATAGTTCTCTTTTT (SEQ ID NO: 24) |
| Sorbitol | SHCLNG- | TRCN000 | Human | CCGGGCCGATACAATCTGTCACCTTCTCGAGAAG |
| dehydrogenase | NM_003104 | 0028082 | | GTGACAGATTGTATCGGCTTTTT (SEQ ID NO: 25) |
| Sorbitol | SHCLNG- | TRCN000 | Human | CCGGGCCAATCGGGATGGTCACTTTCTCGAGAAA |
| dehydrogenase | NM_003104 | 0028052 | | GTGACCATCCCGATTGGCTTTTT (SEQ ID NO: 26) |
| Sorbitol | SHCLNG- | TRCN000 | Human | CCGGCGTCCAAGTCTGTGAATGTAACTCGAGTTA |
| dehydrogenase | NM_003104 | 0028106 | | CATTCACAGACTTGGACGTTTTT (SEQ ID NO: 27) |

The term “microRNA” as used herein refers to a small, non-coding RNA molecule (containing about 22 nucleotides) found in plants, animals and some viruses that functions in RNA silencing and post-transcriptional regulation of gene expression. Gene silencing may occur either via mRNA degradation or preventing mRNA from being translated. miRNAs resemble the siRNAs, except miRNAs derive from regions of RNA transcripts that fold back on themselves to form short hairpins, whereas siRNAs derive from longer regions of double-stranded RNA. An miRNA oligonucleotide can be constructed using chemical synthesis and/or enzymatic ligation reactions using procedures known in the art. Alternatively, the miRNA oligonucleotide can be transcribed biologically using an expression vector into which a nucleic acid has been subcloned in an antisense orientation (i.e., RNA transcribed from the inserted nucleic acid will be of an antisense orientation to a target nucleic acid of interest).

The term “aptamer” as used herein refers to short single-stranded oligonucleotides or a plurality of said oligonucleotides that bind to target molecules with high affinity, such as a small molecule, protein, nucleic acid, cell, tissue, or organism. Selection of aptamers that specifically bind a target mRNA may be accomplished by any suitable method known in the art, including but not limited to by an in vitro process known as whole Cell-SELEX (Systematic Evolution of Ligands by Exponential enrichment).

In another embodiment, the therapeutic agent is formulated as a pharmaceutical composition prior to administering to a subject, according to techniques known in the art. Pharmaceutical compositions of the disclosure are characterized as being at least sterile and pyrogen-free. As used herein, “pharmaceutical formulations” or “pharmaceutical compositions” include formulations for human and veterinary use.

Methods of Suppressing Liver Metastases

Another aspect of the present disclosure provides a method of treating cancer in a subject in need thereof, the method comprising administering to the subject an effective amount of a therapeutic agent capable of down-regulating and/or inhibiting a fructose enzyme or transporter in a cell of the subject such that the cancer growth is suppressed.

The terms “treating” or “treatment” as used herein refers to both therapeutic treatment and prophylactic or preventative measures. It refers to preventing, curing, reversing, attenuating, alleviating, minimizing, suppressing, or halting the deleterious effects of a disease state, disease progression, disease causative agent (e.g., bacteria or viruses), or other abnormal condition. In some embodiments, treating cancer refers to delaying metastatic onset or deterring metastatic growth of a cancer cell (e.g., a CRC cell). In other embodiments, treating cancer refers to suppressing the metastatic growth of a cancer cell (e.g., a CRC cell that has metastasized to the liver).

The term “suppress” as used herein with respect to suppressing cancer growth refers to halting, reversing, or lessening the effects of a disease state and/or halting, reversing, or shrinking the size of a tumor.

The terms “effective amount” and “therapeutically effective amount” as used herein refers to an amount of a therapeutic agent sufficient to effect beneficial or desirable biological and/or clinical results. Such response may be a beneficial result, including, without limitation, amelioration, reduction, prevention, suppression, or elimination of symptoms of a disease or disorder. Therefore, the total amount of each active component of the therapeutic agent is sufficient to demonstrate a meaningful benefit in the patient, including, but not limited to, suppressing liver metastases. A “therapeutically effective amount” may be administered through one or more preventative or therapeutic administrations. When the term “therapeutically effective amount” is used in reference to a single agent, administered alone, the term refers to that agent alone, or a composition comprising that agent and one or more pharmaceutically acceptable carriers, excipients, adjuvants, or diluents. When applied to a combination, the term refers to combined amounts of the active agents that produce the therapeutic effect, or composition(s) comprising the agents, whether administered in combination, consecutively, or simultaneously. The exact amount required will vary from subject to subject, depending, for example, on the species, age, and general condition of the subject; the severity of the condition being treated; and the mode of administration, among other factors known and understood by one of ordinary skill in the art. An appropriate “effective” amount in any individual case may be determined by one of ordinary skill in the art. Thus, a “therapeutically effective amount” will typically fall in a relatively broad range that can be determined through routine trials.

The therapeutic agents described herein can be administered by any suitable route of administration. In certain embodiments, the therapeutic agent is administered intravenously, subcutaneously, transdermally, intradermally, intramuscularly, orally, transcutaneously, intraperitoneally (IP), intravaginally, or via intrahepatic or cecal injection.

The therapeutic agent of the disclosure can be administered to the subject either naked or in conjunction with a delivery reagent. Examples of delivery reagents for administration in conjunction with the therapeutic agent include, but are not limited to, Mirus Transit TKO lipophilic reagent, lipofectin, lipofectamine, cellfectin, polycations (e.g., polylysine), micelles, PEGylated liposome or nanoparticles, oligonucleotide nanoparticles, cyclodextrin polymer (CDP)-based nanoparticles, biodegradable polymeric nanoparticles formulated with poly-(D,L-lactide-co-glycolide) (PLGA), Poly-lactic acid (PLA), or N-(2-hydroxypropyl)methacrylamide (HPMA), lipid nanoparticles (LNP), stable nucleic acid lipid particles (SNALP), vitamin A coupled lipid nanoparticles, and combinations thereof.

One skilled in the art can also readily determine an appropriate dosage regimen for administering the therapeutic agents to a given subject.

The terms “subject” and “patient” are used interchangeably herein to refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient suffering from, or at risk of developing, a metastatic cancer (e.g., metastatic liver cancer).

In some embodiments of the methods of the disclosure, the cancer is a liver cancer. In other embodiments, the cancer is a metastatic cancer (e.g., metastatic liver cancer).

In some embodiments of the methods of the disclosure, the fructose enzyme or fructose transporter is selected from aldolase B (ALDOB), ketohexokinase (KHK), GLUT5, GLUT2, aldose reductase, or sorbitol dehydrogenase, or combinations thereof.

In some embodiments, the therapeutic agent used in the methods described herein is an RNAi polynucleotide, a small molecule, or an antibody. In some embodiments, the RNAi polynucleotide can be small interfering RNA (siRNA), short hairpin RNA (shRNA), and microRNA (miRNAs) oligonucleotides.

In other embodiments, the therapeutic agent used in the methods described herein is an RNAi polynucleotide capable of knocking down ALDOB (e.g., SEQ ID NOS:1-2), an RNAi polynucleotide capable of knocking down KHK (e.g., SEQ ID NOS:3-7), an RNAi polynucleotide capable of knocking down aldose reductase (e.g., SEQ ID NOS:18-22), an RNAi polynucleotide capable of knocking down sorbitol dehydrogenase (e.g., SEQ ID NOS:23-27), an RNAi polynucleotide capable of knocking down GLUT2 (e.g., SEQ ID NOS:13-17), or an RNAi polynucleotide capable of knocking down GLUT5 (e.g., SEQ ID NOS:8-12), or combinations thereof.

In other embodiments, the therapeutic agent is a small molecule inhibitor of aldolase B (ALDOB), ketohexokinase (KHK), aldose reductase, sorbitol dehydrogenase, GLUT5, or GLUT2.

In some embodiments, the therapeutic agent blocks de novo fructose synthesis in a cell (e.g., a cancer cell and/or a non-cancer cell) of the subject. As used herein, the term “de novo fructose synthesis” refers to fructose that is formed by a chemical reaction in the cell.

In some embodiments, the small molecule is an inhibitor of KHK. Small molecule inhibitors of KHK include, but are not limited to, pyrimidinopyrimidine 1, indazole 2, pyrrolopyridine 7, pyrrolopyridine 9, pyrrolopyridine 10, pyridine 8, pyridine 11, pyridine 12, any combinations thereof, and any salts, esters, isomers, and derivatives thereof. In other embodiments, the small molecule is pyridine 12.

In some embodiments, the small molecule is an inhibitor of aldose reductase. Small molecule inhibitors of aldose reductase include, but are not limited to, alrestatin, epairestat, fidarestat, imirestat, lidoestat, minalrestat, ponalrestat, ranirestat, salfredin B₁₁, sorbinil, tolrestat, zenarestat, and zopolrestat, any combinations thereof, and any salts, esters, isomers, and derivatives thereof.

In some embodiments, the small molecule is an inhibitor of sorbitol dehydrogenase. Small molecule inhibitors of sorbitol dehydrogenase include, but are not limited to, CP-470711 (SDI-711) and WAY-135706, and any salts, esters, isomers, and derivatives thereof.

In some embodiments, the small molecule is an inhibitor of GLUT5. Small molecule inhibitors of GLUT5 include, but are not limited to, N-[4-(methylsulfonyl)-2-nitrophenyl]-1,3-benzodioxol-5-amine (MSNBA), glyco-1,3-oxazolidin-2-thiones (OZT) (e.g., D-arabinose-OZT, L-arabinose-OZT, D-xylose-OZT, D-ribose-OZT, D-fructose-Bn-OZT, D-fructose-allyl-OZT, D-fructose-OZT, L-sorbose-allyl-OZT, L-sorbose-Bn-OZT, and L-sorbose-OZT), and glyco-1,3-oxazolidin-2-ones (OZO) (e.g., D-fructose-Bn-OZO, D-fructose-OZO, L-sorbose-Bn-OZO, and L-sorbose-OZO), and any salts, esters, isomers, and derivatives thereof.

In other embodiments, the small molecule is an inhibitor of GLUT2. Small molecule inhibitors of GLUT2 include, but are not limited to, ertugliflozin, empagliflozin, canagliflozin, dapagliflozin, ipragliflozin, phloretin, myricetin, fisetin, quercetin, isoquercitrin, sappanin-type (SAP) homoisoflavonoids, and any salts, esters, isomers, and derivatives thereof.

In other embodiments, the therapeutic agent is an antibody against aldolase B (ALDOB), ketohexokinase (KHK), aldose reductase, sorbitol dehydrogenase, GLUT5, or GLUT2. In other embodiments, the therapeutic agent is a neutralizing antibody against GLUT5 (e.g., AGT-025, ab36057, ab41533, ab113931, ab87847, ab190555, ab111299, and OTI20E1) or GLUT2 (e.g., AGT-022, 600-401-GN3, LS-B15821, and LS-B4177).

In some embodiments, the method of treating cancer in a subject in need thereof further comprises restricting the dietary intake of fructose in the subject. Dietary intake of fructose refers to the fructose that the subject consumes (e.g., by eating food or being fed intravenously) that contains sucrose or fructose. In some embodiments, restricting the dietary intake of fructose, as used herein, refers to reducing the amount of fructose that the subject normally consumes on a daily, weekly, monthly, or yearly basis during the duration of the cancer treatment. In some embodiments, restricting the dietary intake of fructose refers to eliminating fructose completely from the diet of the subject (e.g., a diet devoid of fructose) for the duration of the cancer treatment. In some embodiments, restricting the dietary intake of fructose continues for a duration after the cancer treatment has ended (e.g., days, weeks, months, years after the cancer treatment has ended). An example of a fructose-high, fructose-restricted and regular diet for mice is shown in Example 7.

In some embodiments, restricting the dietary intake of fructose, implemented either alone or in combination with treatment with a therapeutic agent that is capable of down-regulating and/or inhibiting a fructose enzyme or fructose transporter in a subject suffering from liver metastases results in suppressed CRC tumors of the liver and can be more effective than 5-Fluorouracil or Oxaliplatin, both of which are frontline chemotherapy for advanced and metastatic CRC (Alberts, S. R., et al., (2005); Andre, T., et al., (2004)).

Another aspect of the present disclosure provides a method of treating cancer in a subject in need thereof, the method comprising administering to the subject an effective amount of a therapeutic agent capable of blocking de novo fructose synthesis in a cell of the subject such that the cancer growth is suppressed. In some embodiments, the cell is a cancer cell. In other embodiments, the cell is a non-cancer cell.

In some embodiments, the therapeutic agent is a small molecule or antibody inhibitor of ALDOB, KHK, aldose reductase, sorbitol dehydrogenase, GLUT5, or GLUT2.

Yet another aspect of the present disclosure provides a method of suppressing cancer growth in a subject in need thereof, the method comprising down-regulating and/or inhibiting a fructose enzyme in a cell of the subject. In some embodiments, the fructose enzyme or fructose transporter is selected from aldolase B (ALDOB), aldose reductase, sorbitol dehydrogenase, ketohexokinase (KHK), GLUT5, or GLUT2. In some embodiments, the cell is contacted with a small molecule including, but not limited to, any of pyrimidinopyrimidine 1, indazole 2, pyrrolopyridine 7, pyrrolopyridine 9, pyrrolopyridine 10, pyridine 8, pyridine 11, pyridine 12, alrestatin, epairestat, fidarestat, imirestat, lidoestat, minalrestat, ponalrestat, ranirestat, salfredin B₁₁, sorbinil, tolrestat, zenarestat, zopolrestat, CP-470711 (SDI-711), N-[4-(methylsulfonyl)-2-nitrophenyl]-1,3-benzodioxol-5-amine (MSNBA), D-arabinose-OZT, L-arabinose-OZT, D-xylose-OZT, D-ribose-OZT, D-fructose-Bn-OZT, D-fructose-allyl-OZT, D-fructose-OZT, L-sorbose-allyl-OZT, L-sorbose-Bn-OZT, and L-sorbose-OZT, D-fructose-Bn-OZO, D-fructose-OZO, L-sorbose-Bn-OZO, and L-sorbose-OZO), ertugliflozin, empagliflozin, canagliflozin, dapagliflozin, ipragliflozin, phloretin, myricetin, fisetin, quercetin, isoquercitrin, or a sappanin-type (SAP) homoisoflavonoid, and any salts, esters, isomers, and derivatives thereof, and combinations thereof. In other embodiments, the cell is contacted with an antibody including, but not limited to, an anti-ALODB antibody, an anti-KHK antibody, an anti-aldose reductase antibody, and anti-sorbitol dehydrogenase antibody, an anti-GLUT2 (e.g., AGT-022, 600-401-GN3, LS-B15821, or LS-B4177) antibody, or an anti-GLUT5 (e.g., AGT-025, ab36057, ab41533, ab113931, ab87847, ab190555, ab111299, OTI20E1) antibody, and combinations thereof.

In other embodiments, the cell is contacted with an RNAi polynucleotide (e.g., via transfection) including, but not limited to, an RNAi polynucleotide capable of knocking down ALDOB (e.g., SEQ ID NOS:1-2), an RNAi polynucleotide capable of knocking down KHK (e.g., SEQ ID NOS:3-7), an RNAi polynucleotide capable of knocking down aldose reductase (e.g., SEQ ID NOS:18-22), an RNAi polynucleotide capable of knocking down sorbitol dehydrogenase (e.g., SEQ ID NOS:23-27), an RNAi polynucleotide capable of knocking down GLUT2 (e.g., SEQ ID NOS:13-17), or an RNAi polynucleotide capable of knocking down GLUT5 (e.g., SEQ ID NOS:8-12), or combinations thereof.

The following examples are offered by way of illustration and not by way of limitation.

### Example 1: Meta-analysis of Clinical CRC Liver Metastases

To investigate the differential transcriptomic signatures, four NCBI Gene Expression Omnibus (GEO) datasets including clinical samples of normal colon, primary CRC tumor and liver metastases were selected.

Meta-Analysis

Four NCBI Gene Expression Omnibus (GEO) databases that contain transcriptomic profiling of 102 normal colon, 254 primary CRC, and 111 CRC liver metastasis samples were identified (Barrett, T., et al., (2013) Nucleic Acids Res, 41:D991-95; Del Rio, M., et al., (2013) PLos One, 8:e74599; Pizzini, S., et al., (2013) BMC Genomics, 14:589; Sheffer, M., et al., (2009) Proc Natlk Acad Sci USA, 106:7131-7136; Stange, D. E., et al., (2010) Gut, 59:1236-1244) (Table 2). Within these four datasets, 90 matched samples (normal colon, primary CRC, liver metastasis) from 30 Stage IV CRC patients from these datasets were selected and processed by standard GEO2R analysis. The microarray data were then processed by quantile normalization and log 2 transformation. Paired differential analysis were performed using the R package The Linear Models for Microarray Data (Limma) (Ritchie, M. E., et al., (2015) Nucleic Acids Res 43:e47).

TABLE 2
Information of NCBI:GEO databases used in this study.

| | | | Normal | Primary | Liver |
|---|---|---|---|---|---|
| Title | Platform | GEO ID | colon | CRC | mets |
| Expression Profile of | Illumina human-6 | GSE14297 | 7 (7) | 18 (7) | 18 (7) |
| Primary Colorectal | v2.0 expression | | | | |
| Cancers and associated | beadchip | | | | |
| Liver Metastases | | | | | |
| Impact of miRNAs | [HuEx-1_0-st] | GSE35834 | 23 (9) | 30 (9) | 27 (9) |
| modulation on | Affymetrix Human | | | | |
| regulatory networks and | Exon 1.0 ST Array | | | | |
| pathways involved in | | | | | |
| colon cancer and | | | | | |
| metastasis development | | | | | |
| Expression data from | [HG-U133A] | GSE41258 | 54 (4) | 186 (4) | 47 (4) |
| colorectal cancer | Affymetrix Human | | | | |
| patients | Genome U133A | | | | |
| | Array | | | | |
| Specific extracellular | [HG-U133A] | GSE49355 | 18 (10) | 20 (10) | 19 (10) |
| matrix remodeling | Affymetrix Human | | | | |
| signature of colon | Genome U133A | | | | |
| hepatic metastases | Array | | | | |

( ): number of matched samples

Statistical Analysis

Limma was used for the differential analyses of transcriptomic data and metabolomic data. The global metabolic maps were generated from the KEGG mapper toolbox using Interactive Pathway Explorer (iPATH2) (Yamada, T. et al., (2011) Nucleic Acid Res, 39:W412-415). Gene Set Enrichment Analysis (GSEA) of the clinical microarray data was performed using the GSEA software (Subramanian, A., et al., (2005) Proc Natl Acad Sci USA, 102:15545-15550). Heatmaps and hierarchal clustering were performed using Morpheus available from (https://software.broadinstitute.org/morpheus/).

Results

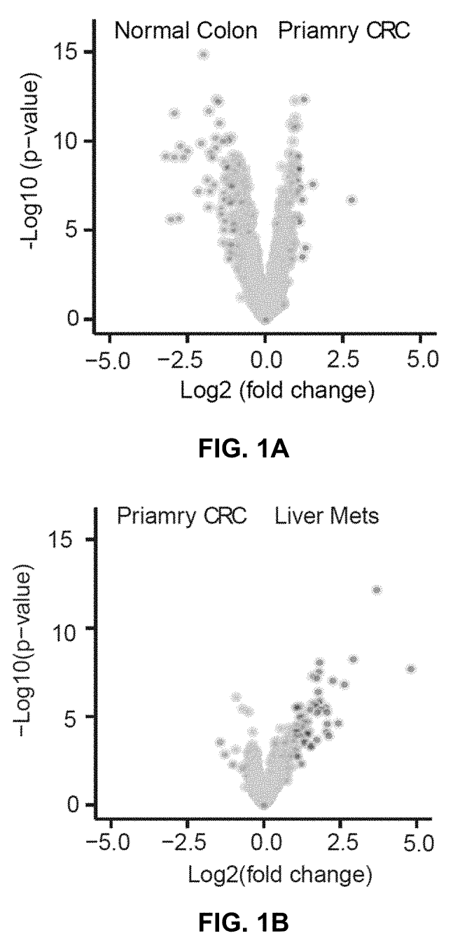

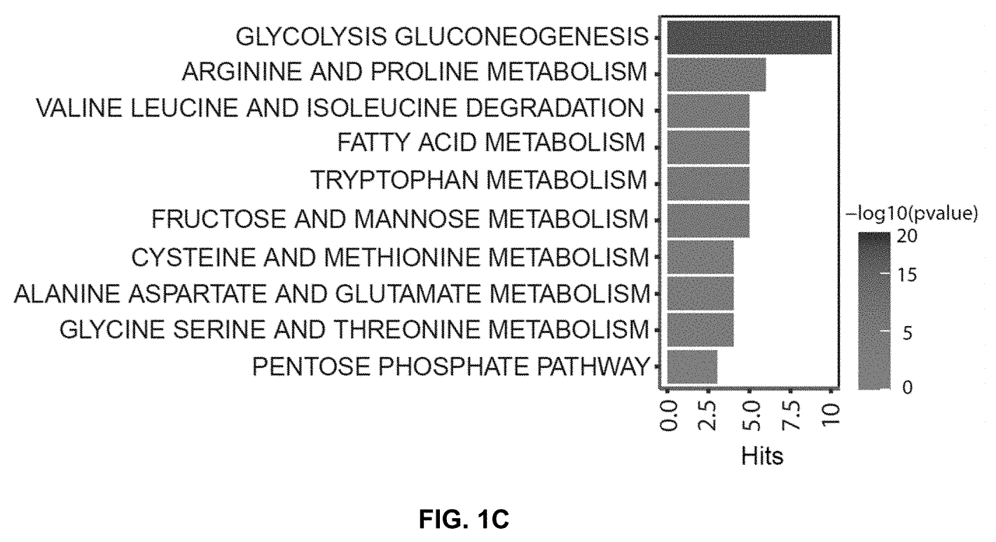

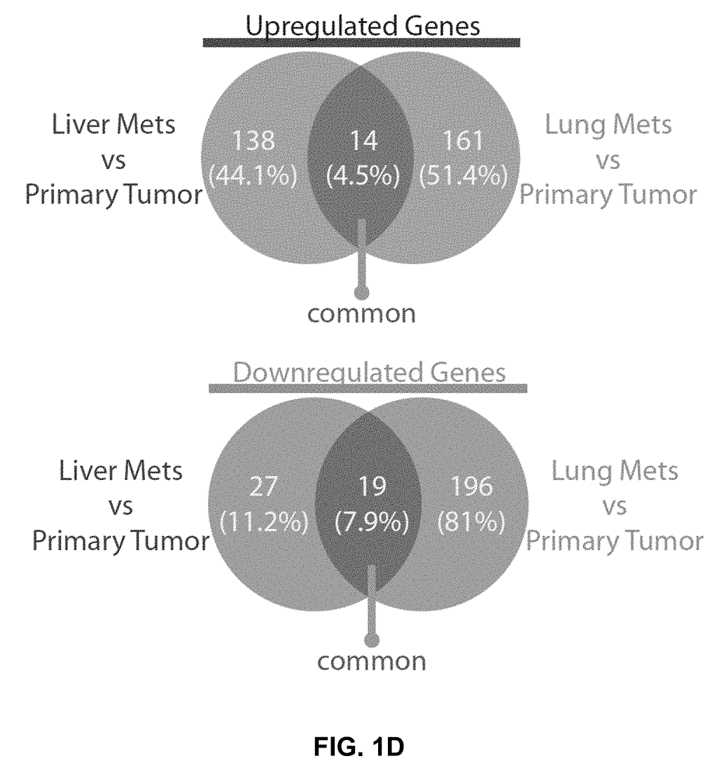

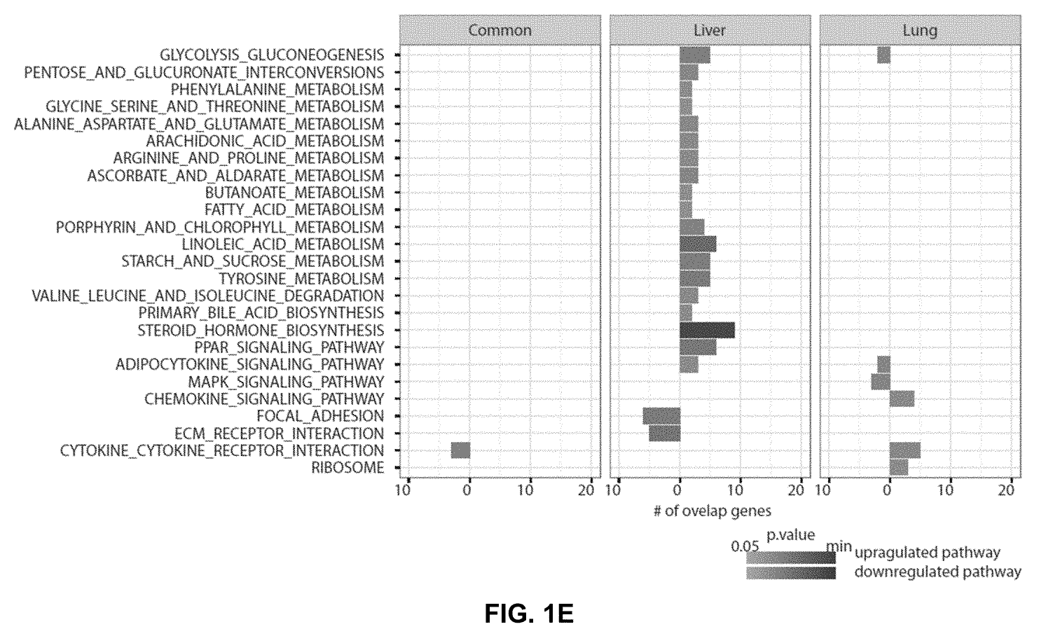

Paired differential analysis comparing matching normal colon tissues and primary CRC samples identified a set of differentially expressed genes, about 9.5% of which are involved in metabolic pathways (FIG. 1A). In comparison, 23% of differentially expressed genes between matching primary CRC and liver metastasis are metabolism related, and more than 90% of the differentially expressed metabolic genes are upregulated in liver metastasis comparing to primary CRC (FIG. 1B). Gene Set Enrichment Analysis (GSEA) between primary CRC and liver metastasis samples suggested highly altered activity levels in certain metabolic pathways (FIG. 1C) including glycolysis/gluconeogenesis, amino acid metabolism, fructose and mannose metabolism. This meta-analysis across independent clinical datasets and platforms suggests potential metabolic alternation between primary CRC and liver metastasis.

Analysis of 186 primary tumor samples, 47 liver metastatic samples, and 20 lung metastatic (unmatched) samples in the GSE41258 CRC dataset (Sheffer, M., et al., (2009) Proc Natl Acad Sci USA, 106:7131-7136) (the only set available containing adequate CRC lung metastasis) suggests that liver metastases and lung metastases have distinct transcriptomic signatures. There are few overlap between genes up or down-regulated in liver vs. lung metastases (FIG. 1D). Pathway analysis indicates that more metabolic pathways are upregulated in liver metastases compared to lung metastases (FIG. 1E).

### Example 2: Integrated Metabolomics and Transcriptomics Analysis of a CRC Liver Metastasis Model

Next, an in vivo CRC metastatic model was used by injecting mcherry- and luciferase-labeled HCT116 cells into NOD/SCID mice to study how microenvironment affect CRC cell metabolism (Bu, P., et al., (2015), Nat Commun 6:6879).

Mice and Treatments

All animal experiments were approved by The Cornell Center for Animal Resources and Education (CARE) and followed the protocol (2009-0071 and 2010-0100). 6-8 week old NOD/SCID mice and BALB/c mice were used throughout the study.

Cell Lines, Lentiviral Vector Constructs and Infection

Human CRC cell line HCT116, patient derived xenograft human CRC cell line CRC119 and CRC57 and BALB/c mouse colon cancer cell line CT26 were used in the study (Table 3). The cell lines were grown in RPMI 1640 complete medium with 10% FBS and 1% penicillin-streptomycin solution.

TABLE 3
Information of patients who CRC119 and CRC57
cell lines were derived from.

| | | | Metastatic | | |
|---|---|---|---|---|---|
| Cell line | Gender | Primary site | site | Differentiation | Stage |
| CRC119 | F | colon | liver | moderate | IV |
| CRC57 | M | colon | liver | moderate | IV |

The dual mCherry and luciferase reporter was constructed using lentiviral pFUW backbone (Addgene). Briefly, the vector was cut by restriction enzymes BamHI and EcoRI. The firefly luciferase-E2A-mCherry was amplified and connected by overlapping PCR and cut by BamHI and EcoRI. E2A is a self-cleaving peptide sequence. Immediately after luciferase-E2A-mCherry is translated into a fusion protein, it splits into separate luciferase and mCherry inside mammalian cells.

Metabolomics

Primary cecum tumors and liver metastases were harvested from the mice. The tissues were rinsed with water and immediately transferred to a tube and placed in liquid nitrogen. Frozen tissue was minced, weighed, and 5 mg was dissolved in 80% methanol. Samples were centrifuged at maximum speed at 4° C. The resulting supernatant was transferred to a fresh tube and dried using SpeedVac concentrator (SPD131DDA, Thermo Scientific). Pellets were dissolved in 50 ul water, and diluted with equal volume of acetonitrile:methanol (1:1, v:v) solution. Tubes were centrifuged again for 5 minutes to eliminate insoluble pellets, and 5 ul was injected into mass spectrometry tubes to measure polar metabolites using LC-MS as previously described (Liu, X., et al., (2014) Journal of Visualized Experiments: JoVE 51358).

Bioenergetics Assay

Liver metastases cells were purified by FACS based on mCherry expression and seeded into 24-well Seahorse XF24 cell culture microplate at a density of 40,000 cells per well in 2 steps. Frist, 100 ul of growth medium was added, cells were incubated for 4 hours to ensure the formation of a monolayer, and then another 150 ul of growth medium was added. The next day, medium was switched to XF Base medium without supplements and in the absence of glucose and glutamine. Fructose (11 mM) was added to port A for injection. Both cell plate and fructose solution were incubated at 37° C. without CO2 for 1 hour prior to assay. Baseline OCR and ECAR measurements were recorded before and after Fructose injection into the medium.

Integrated Analysis of RNA-Seq and Metabolomics Data

Cancer cells were purified by FACS based on mCherry expression, and the transcriptomes were profiled using Illumina HiSeq2000 at The Genomics Core of Weill Cornell Medical College. TopHat2 (Kim, D., et al., (2013) Genome Biol, 14:R36) and HTSeq (Anders, S., et al., (2015) Bioinformatics 31:166-169) were used for RNA-seq data analysis with UCSC hg19 as the reference genome. The differential analysis of the RNA-seq data was performed by using DESeq2 (Love, M. I., et al., (2014) Genome Biol, 15:550). The significant-differential (p value<0.05) genes were selected and integrated with the significant-differential (p value<0.05).

Results

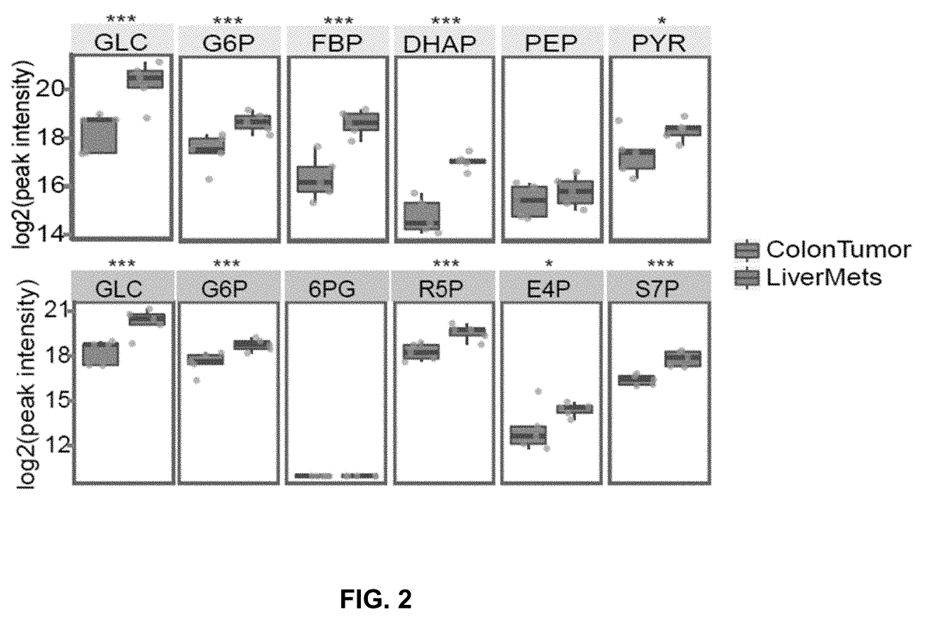

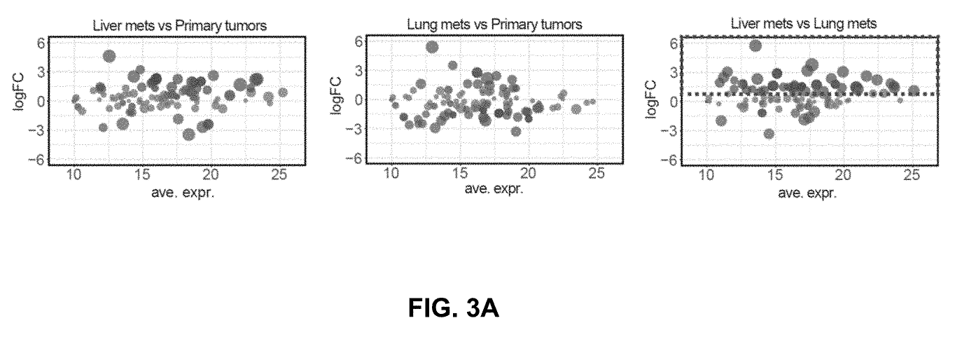

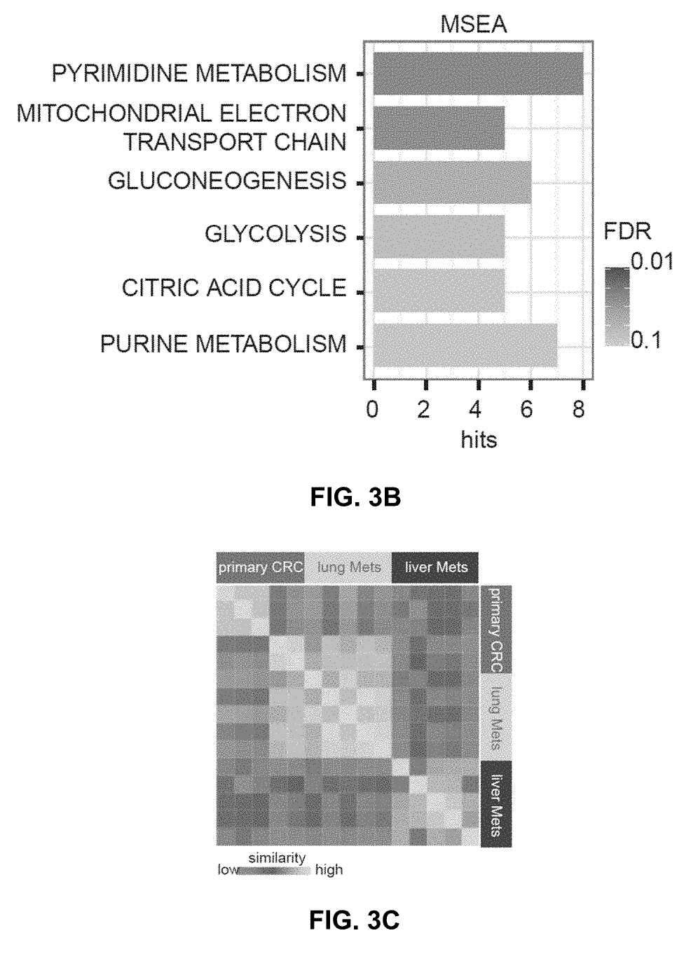

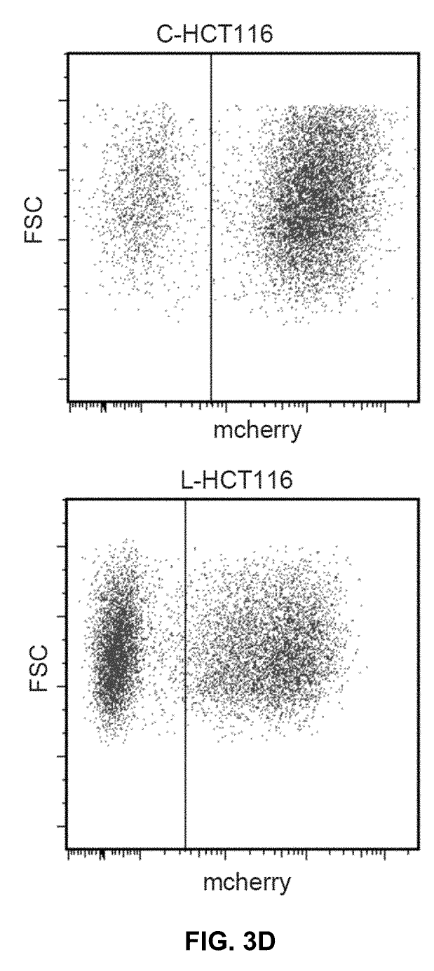

Five weeks after injection, primary, liver metastatic and lung metastatic tumors were harvested and processed for metabolomics using a high-resolution, Q Exactive liquid chromatography-mass spectrometry (LC-MS) platform and bioinformatics analytical workflow (Liu et al., 2014). Data shown in a heatmap of metabolite clusters indicated the presence of distinct metabolite clustering in primary colon tumors vs. metastatic liver tumors as measured by LC-MS based metabolomics. Differential analyses identified metabolites with levels significantly altered in liver metastases compared to primary tumors. For example, metabolites of the glycolysis/gluconeogenesis and pentose phosphate pathways were upregulated in liver metastases (FIG. 2). Differential analysis, metabolite set enrichment analysis (MSEA), and similarity matrix analysis of metabolites suggest that the lung and liver environments regulate CRC cell metabolism differently, consistent with transcriptomic analyses of the clinical GEO datasets (FIG. 3A-3C).

Metabolite levels do not necessarily indicate pathway activities, so RNA-seq was performed to measure expression levels of the involved metabolic enzymes. To remove stromal cells, HCT116 cells were purified from ceca and livers of tumor-bearing mice based on mCherry expression using fluorescence-activated cell sorting (FACS) (FIG. 3D). RNA-seq measurements were then carried out on the purified CRC cells, and a heatmap was generated of RNA-seq analysis from isolated cells from FIG. 3D.

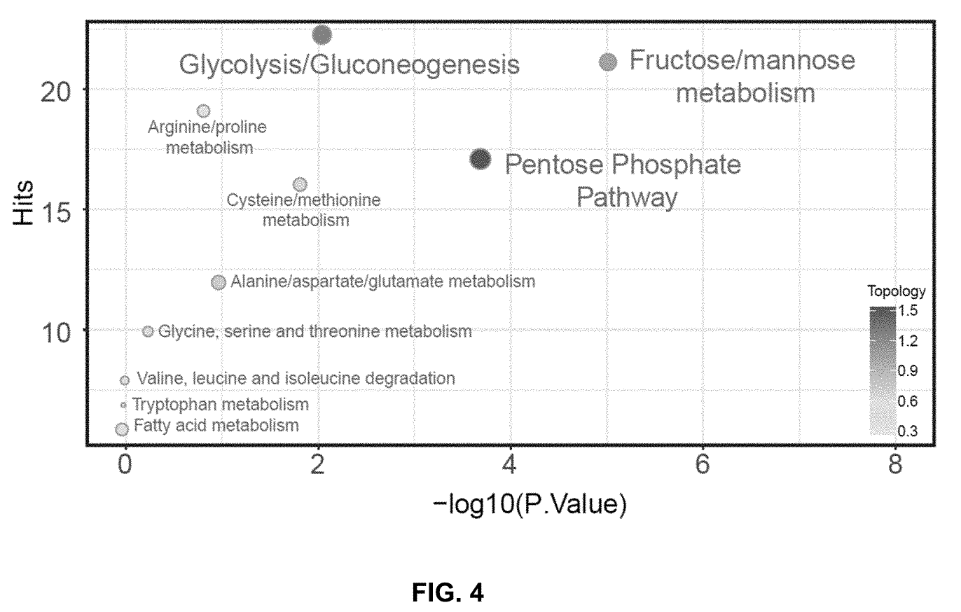

Integrated analysis of transcriptomics and metabolomics data identified metabolic pathways that were likely altered in CRC cells from liver metastases compared to primary tumors, which were further validated by the GEO datasets to highlight clinically relevant pathways (FIG. 4). The differential (p value<0.05) genes from RNAseq and differential metabolites from metabolomics were integrated and viewed in a comprehensive enrichment metabolic map using iPath2 (Letunic, I., et al., (2008) Trends Biochem Sci, 33:101-103) based on KEGG metabolic map.

For example, glycolysis, gluconeogenesis, fructose metabolism, and pentose phosphate pathways seem to be upregulated in CRC cells from liver. Pathways that only contained alterations in metabolite levels but not in enzyme expression were not included, because the variation could have been contributed by stromal cells instead of CRC cells in the lesion. The analysis suggests that CRC cells in the liver have metabolic alterations compared to their counterparts in the primary tumor.

### Example 3: ALDOB is Up-regulated in Liver Metastases

To further confirm ALDOB up-regulation in CRC liver metastases, microarray analysis was conducted.

ALDOB was among the top metabolic genes identified by our meta-analysis of matched samples in the GEO dataset (Table 4).

TABLE 4
Top metabolic genes up-regulated in liver mets vs. primary CRCs

| Gene. symbol | logFC | P. Value | adj. P. Val |
|---|---|---|---|
| G6PC | 2.13 | 1.26E−04 | 6.17E−03 |
| ALDOB | 2.08 | 2.70E−05 | 1.85E−03 |
| ADH1B | 2.03 | 3.11E−06 | 3.07E−04 |
| HPD | 1.88 | 4.08E−06 | 3.85E−04 |
| GATM | 1.82 | 8.97E−09 | 2.65E−06 |
| AOX1 | 1.80 | 3.11E−08 | 7.12E−06 |

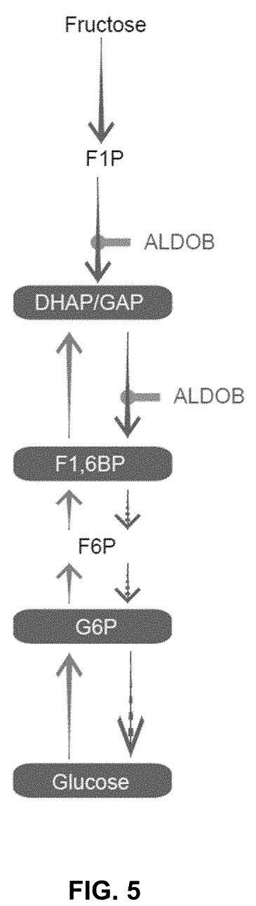

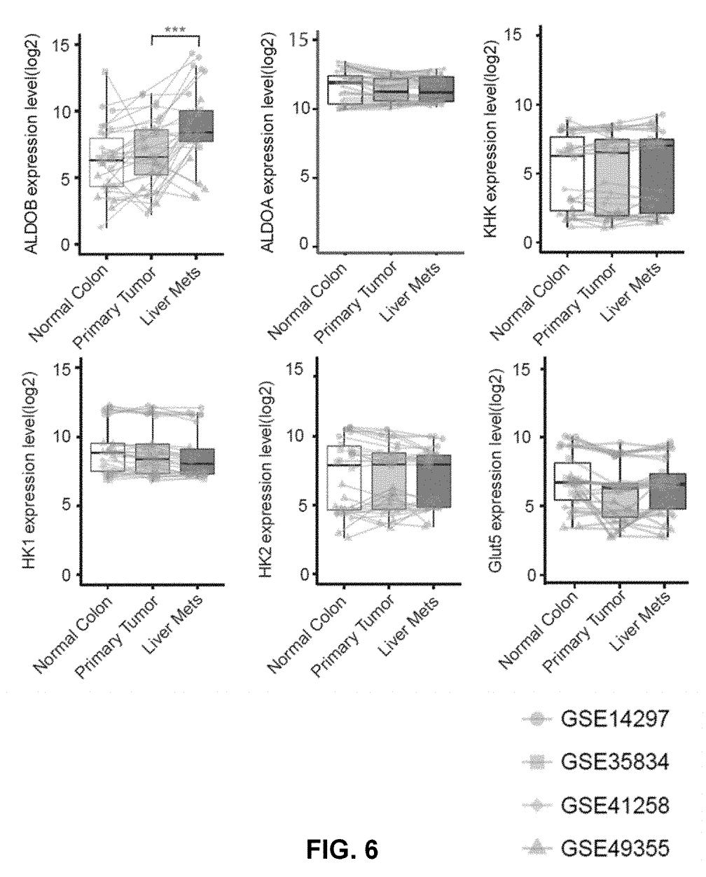

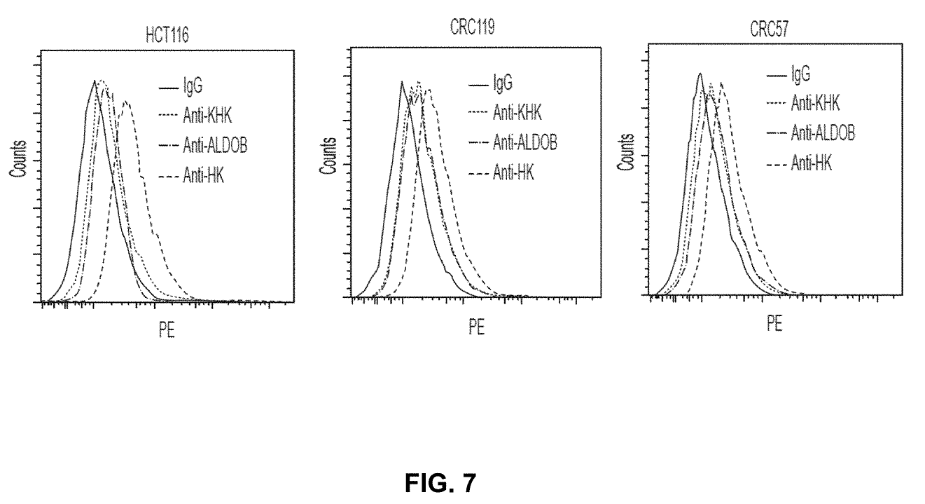

The metabolites involved with ALDOB as shown in FIG. 5 were significantly up-regulated in liver metastases (FIG. 2). A more detailed paired differential analysis of the matched normal colon, primary CRC, and liver metastasis samples from the 30 patients (90 samples in total) in GEO confirmed that ALDOB is consistently up-regulated in liver metastasis compared to matched normal colon and primary CRC, while aldolase A (the aldolase isoform that is not specific to fructose metabolism), KHK, HK1, HK2 and GLUT5 (fructose transporter) levels remain largely unchanged (FIG. 6). Analysis of the unmatched 186 primary, 47 liver, and 20 lung CRC samples from GSE41258 confirmed that ALDOB is up-regulated in liver metastasis but not in lung metastasis, while aldolase A, KHK, HK1, HK2 and GLUT5 levels remain unchanged among primary and metastatic samples (FIG. 7).

Microarray Analysis

DNA microarray measurements were meticulous collected and carried out on 39 primary colon carcinoma, 74 liver metastasis, and 8 lung metastasis samples from stage IV CRC patients at Duke Oncology Center (Table 5).

TABLE 5
Information of CRC patients included in the microarray.

| Patient ID | Primary Site | Metastatic Site | Gender | Stage |
|---|---|---|---|---|
| MET_CRC002D | Colon | Liver | Female | IV |
| MET_CRC007A | Colon | Liver | Male | IV |
| MET_CRC011 | Colon | Liver | Male | IV |
| MET_CRC015 | Colon | Liver | Female | IV |
| MET_CRC019 | Colon | Liver | Male | IV |
| MET_CRC021B | Colon | Liver | Male | IV |
| MET_CRC022 | Colon | Liver | Male | IV |
| MET_CRC024A | Colon | Liver | Female | IV |
| MET_CRC028A | Colon | Liver | Female | IV |
| MET_CRC033 | Colon | Liver | Male | IV |
| MET_CRC037A | Colon | Liver | Male | IV |
| MET_CRC039A | Colon | Liver | Male | IV |
| MET_CRC040 | Colon | Liver | Female | IV |
| MET_CRC041 | Colon | Liver | Male | IV |
| MET_CRC044 | Colon | Liver | Female | IV |
| MET_CRC045 | Colon | Liver | Male | IV |
| MET_CRC047 | Colon | Liver | Male | IV |
| MET_CRC049 | Colon | Liver | Male | IV |
| MET_CRC050 | Colon | Liver | Male | IV |
| MET_CRC051 | Colon | Liver | Male | IV |
| MET_CRC056 | Colon | Liver | Male | IV |
| MET_CRC057A | Colon | Liver | Male | IV |
| MET_CRC058 | Colon | Liver | Male | IV |
| MET_CRC060 | Colon | Liver | Female | IV |
| MET_CRC061 | Colon | Liver | Female | IV |
| MET_CRC062 | Colon | Liver | Male | IV |
| MET_CRC063 | Colon | Liver | Male | IV |
| MET_CRC066A | Colon | Liver | Female | IV |
| MET_CRC002D | Colon | Liver | Female | IV |
| MET_CRC067A | Colon | Liver | Female | IV |
| MET_CRC075A | Colon | Liver | Male | IV |
| MET_CRC077A | Colon | Liver | Male | IV |
| MET_CRC078A | Colon | Liver | Male | IV |
| MET_CRC087A | Colon | Liver | Male | IV |
| MET_CRC089A | Colon | Liver | Male | IV |
| MET_CRC092 | Colon | Liver | Female | IV |
| MET_CRC094 | Colon | Liver | Female | IV |
| MET_CRC094B | Colon | Liver | — | IV |
| MET_CRC096 | Colon | Liver | Male | IV |
| MET_CRC097 | Colon | Liver | Male | IV |
| MET_CRC098D | Colon | Liver | Female | IV |
| MET_CRC102A | Colon | Liver | Female | IV |
| MET_CRC103 | Colon | Liver | Female | IV |
| MET_CRC103C | Colon | Liver | — | IV |
| MET_CRC105B | Colon | Liver | Male | IV |
| MET_CRC107A | Colon | Liver | Male | IV |
| MET_CRC108 | Colon | Liver | Male | IV |
| MET_CRC109A | Colon | Liver | Female | IV |
| MET_CRC112 | Colon | Liver | Male | IV |
| MET_CRC118A | Colon | Liver | Female | IV |
| MET_CRC119A | Colon | Liver | Female | IV |
| MET_CRC125A | Colon | Liver | Male | IV |
| MET_CRC128A | Colon | Liver | Male | IV |
| MET_CRC133A | Colon | Liver | Male | IV |
| MET_CRC134A | Colon | Liver | Male | IV |
| MET_CRC145A | Colon | Liver | Male | IV |
| MET_CRC147A | Colon | Liver | Male | IV |
| MET_CRC148A | Colon | Liver | Female | IV |
| MET_CRC149A | Colon | Liver | Male | IV |
| MET_CRC151A | Colon | Liver | Female | IV |
| MET_CRC159 | Colon | Liver | Male | IV |
| MET_CRC160A | Colon | Liver | Male | IV |
| MET_CRC162A | Colon | Liver | Female | IV |
| MET_CRC165A | Colon | Liver | Female | IV |
| MET_CRC172A | Colon | Liver | Male | IV |
| MET_CRC174B | Colon | Liver | Female | IV |
| MET_CRC221A | Colon | Liver | Male | IV |
| MET_CRC230A | Colon | Liver | Male | IV |
| MET_CRC236A | Colon | Liver | Male | IV |
| MET_CRC241A | Colon | Liver | Female | IV |

Their transcriptomes were profiled using the Affymetrix Human Genome U133 Plus 2.0 array and pre-processed by the Affymetirx MAS5 algorithm in the R Affy package.

Results

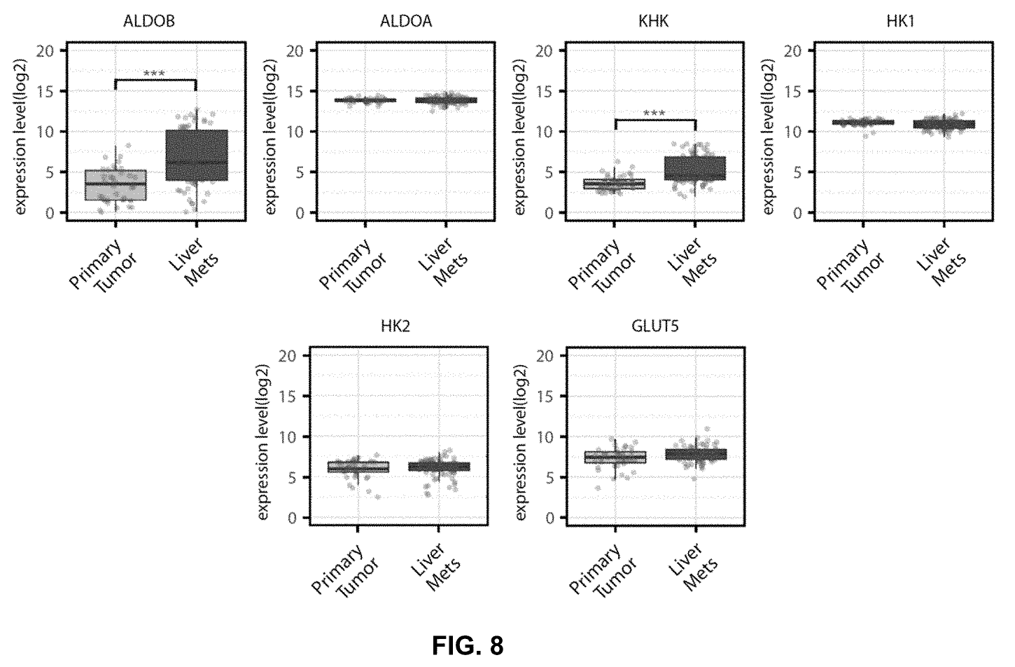

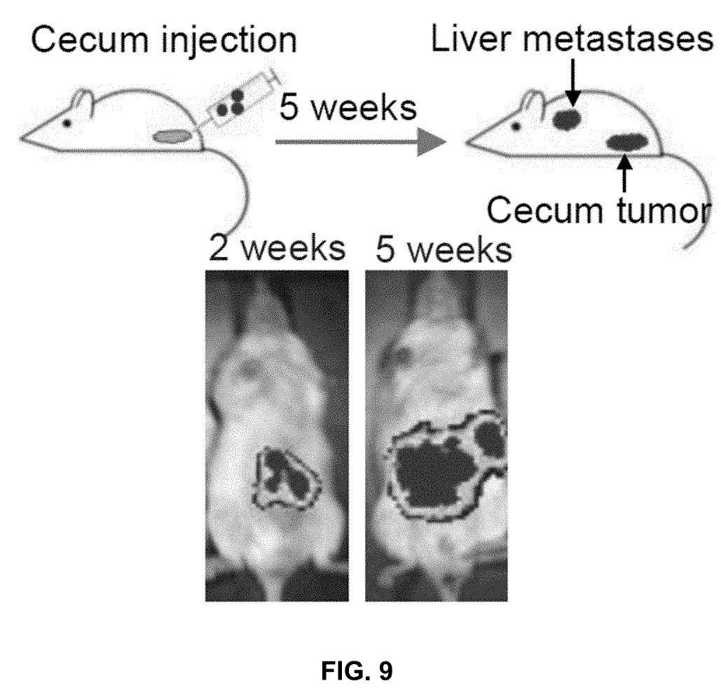

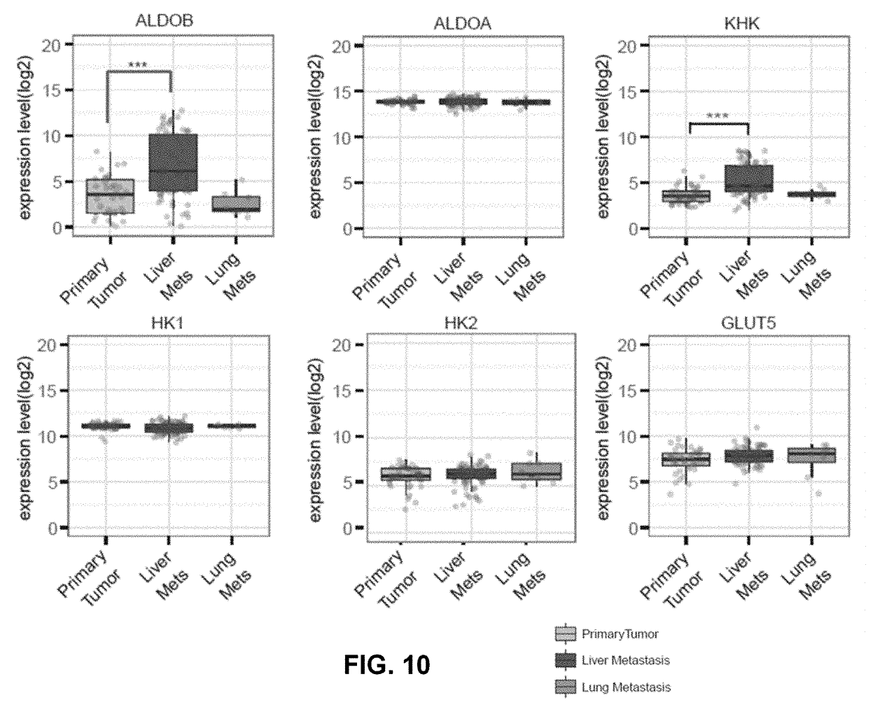

Gene expression analysis indicated upregulation of ALDOB (log(fc)=3.75, p=5.03E-08) in liver metastases compared to primary tumors, while there is no significant difference in the levels of aldolase A, HK1, HK2, and GLUT5 (FIG. 8). In this dataset, KHK level seem to alter somewhat, but not as much as ALDOB. Pathway enrichment analysis of this dataset highlights carbohydrate, glycolysis/gluconeogenesis, and pentose phosphate pathways in liver metastases (FIG. 9). ALDOB and the other enzymes are not upregulated in the lung metastases (FIG. 10), consistent with previous analyses of the GSE41258 dataset, which compared expression levels of ALDOB, ALDOA, KHK, HK1, HK2 and GLUT5 in the GEO dataset (GSE41258) including 186 primary tumor samples, 47 liver metastatic samples, and 20 lung metastatic samples from colorectal cancer patients. Gene Set Enrichment Analysis of genes up-regulated in DNA microarray data analysis on 39 primary colon carcinoma and 74 liver metastasis samples from stage IV CRC patients was also performed.

Lastly, differential analysis (unpaired) was performed for ALDOB in all five datasets (4 GEO datasets and the Duke datasets) respectively, including matched and unmatched samples, which again showed that ALDOB is consistently up-regulated in liver metastases (Table 6).

TABLE 6
Differential expression of ALDOB in liver vs primary CRC
analyzed on the base of five databases

| | | | # of | | |
|---|---|---|---|---|---|
| | | # of Liver | Primary | | |
| Title | GEO ID | mets | CRC | logFC | p. Value |
| Expression data of primary | N/A* | 74 | 39 | 3.75 | 5.03 × 10⁻⁸ |
| CRC and liver metastases | | | | | |
| from Duke Oncology Center | | | | | |
| Expression data from | GSE41258 | 47 | 186 | 2.75 | 4.66 × 10⁻¹⁴ |
| colorectal cancer patients | | | | | |
| Expression Profile of Primary | GSE14297 | 18 | 18 | 1.89 | 1.53 × 10⁻³ |
| Colorectal Cancers and | | | | | |
| associated Liver Metastases | | | | | |
| Impact of miRNAs | GSE35834 | 27 | 30 | 1.48 | 3.22 × 10⁻⁴ |
| modulation on regulatory | | | | | |
| networks and pathways | | | | | |
| involved in colon cancer and | | | | | |
| metastasis development | | | | | |
| Specific extracellular matrix | GSE49355 | 19 | 20 | 2.85 | 1.56 × 10⁻⁴ |
| remodeling signature of colon | | | | | |
| hepatic metastases | | | | | |

*microarray performed in this study.

Taken together, up-regulation of ALDOB in the liver may be common for clinical CRC liver metastases.

### Example 4: Liver Metastases Up-regulate ALDOB

To confirm ALDOB up-regulation in liver metastases, three CRC cell lines—HCT116 and two liver metastasis patient derived xenograft (PDX) cell lines CRC119 and CRC57, were implanted into cecum termini of NOD/SCID mice (Cespedes, M. V., et al., (2007) Am J Pathol, 170:1077-1085; Fu, X. Y., et al., (1991) Proc Natl Acad Sci USA 88:9345-9349) (Table 3).

The cells carried dual-labeled reporter constructs, stably expressing fluorescence protein (mCherry or GFP) and luciferase.

Mice and Treatment

Tumor-bearing mice were treated with 5-Fluorouracil (Sigma, St. Louis, Mo.) at a dose of 100 mg/kg, Oxaliplatin (Sigma, St. Louis, Mo.) at a dose of 6 mg/kg, 2-deoxyglucose (Sigma, St. Louis, Mo.) at a dose of 500 mg/kg or normal saline as vehicle control through intraperitoneal route twice a week. 2×10⁶ cells carrying a luciferase/mCherry or luciferase/GFP vector were injected into the mice for cecum injection model and intrahepatic injection model. 5×10⁵ cells were injected for intravenous injection. Luciferase signal was tracked in vivo using the IVIS luciferase imaging system 200 (Xenogen) for tumor development. Liver metastases were evaluated based on mCherry signals by an OV100 microscope (Olympus) after scarifying the mice.

Western Blot

Western blot was performed as described previously (Bu, P. et al., (2015) Nat Commun, 6:6879). Samples were prepared using the cancer cells purified by FACS based on mcherry expression. Antibodies used included anti-human ALDOB antibody (PA5-30218, 1:2000, Pierce), anti-Hexokinase (C35C4, 1:1000, Cell Signaling), anti-ketohexokinase (4B8, 1:2000, Abcam), anti-Gata6 (1:1000, Abcam) and anti-actin (13E5, 1:1000, Cell Signaling).

Results

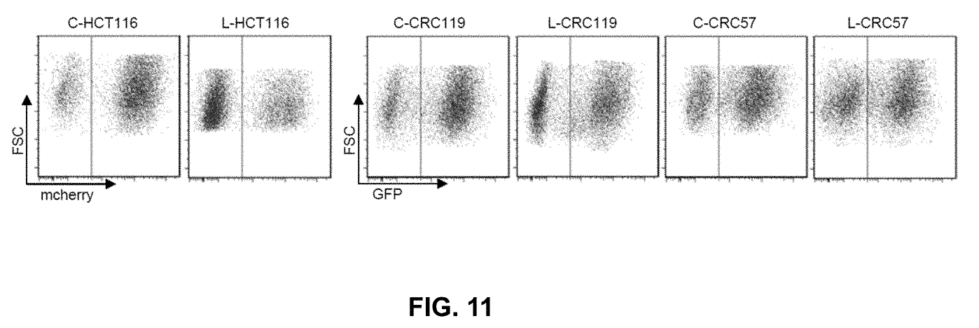

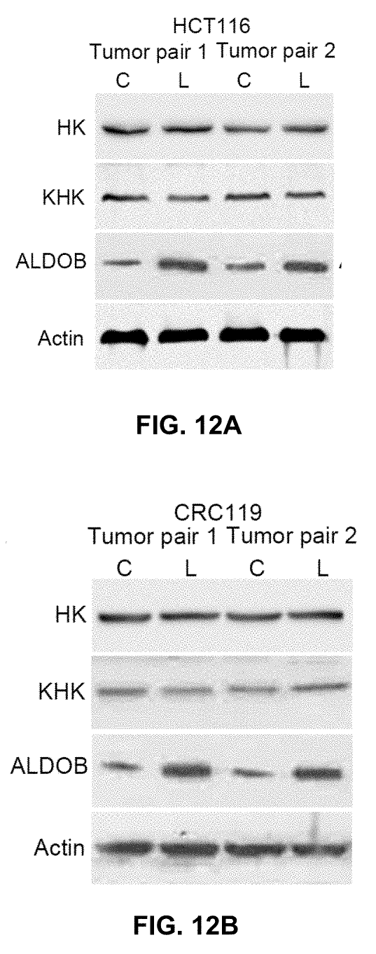

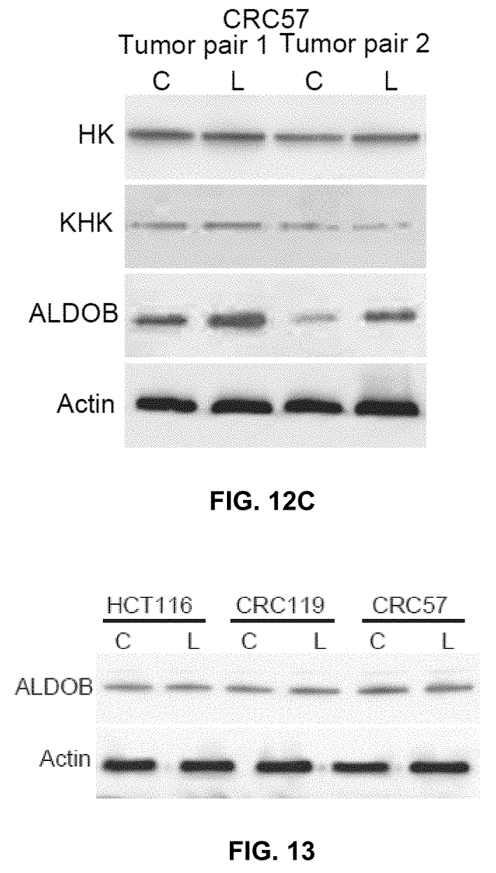

Before cecal injection, FACS analysis showed low levels of KHK and ALDOB, and slightly higher levels of HK in these CRC lines (FIG. 7). After cecal injections, the CRC cells first formed orthotopic tumors within 2 weeks, subsequently developed CRC liver metastases in 5 weeks, when both primary cecal tumors and liver metastases were harvested and CRC cells were isolated by FACS based on fluorescence (FIG. 11 and FIG. 10). Liver metastases had significantly higher ALDOB levels than their primary counterparts, while KHK and HK levels remained largely unchanged (FIGS. 12A-12C). 20%-40% of the mice also developed lung metastases, although ALDOB was not upregulated in lung metastases compared to the primary cecum tumors (FIG. 13A), even after culturing in vitro for 3 days (FIG. 13B).

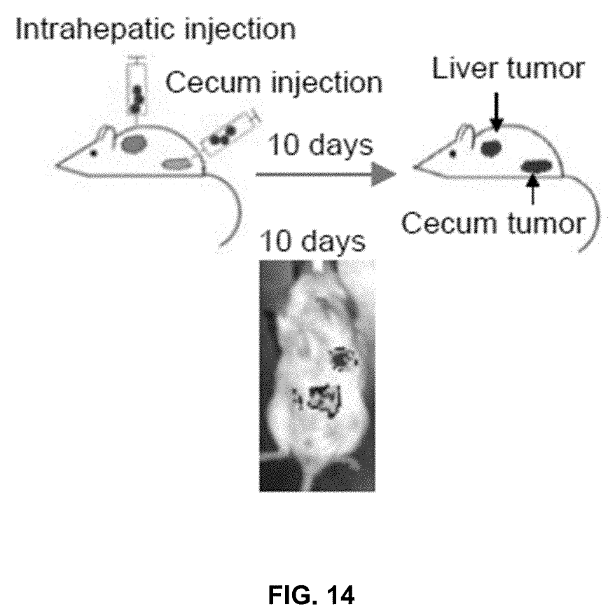

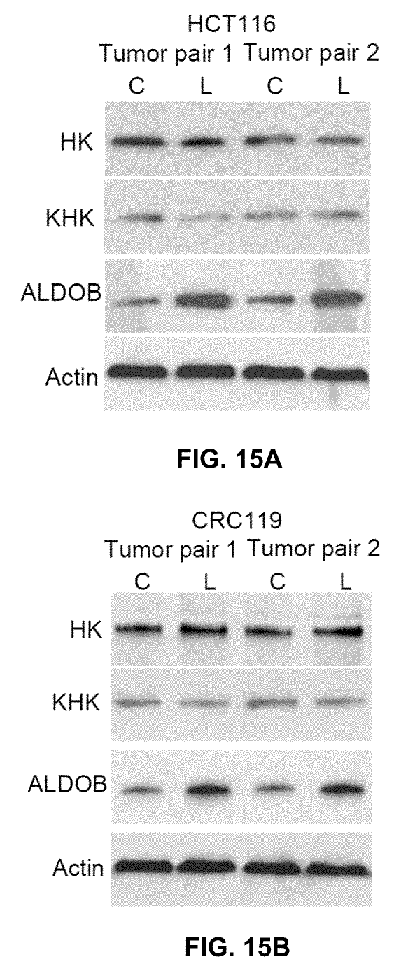

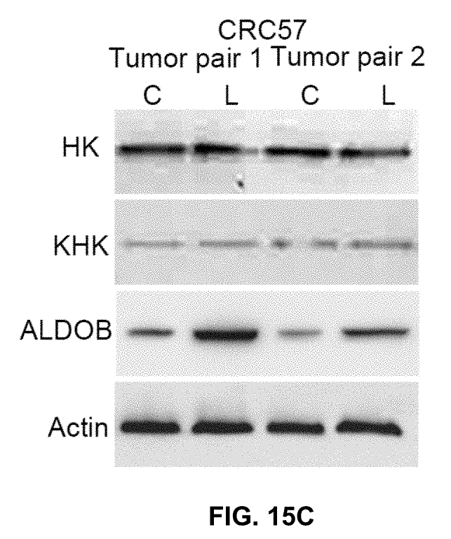

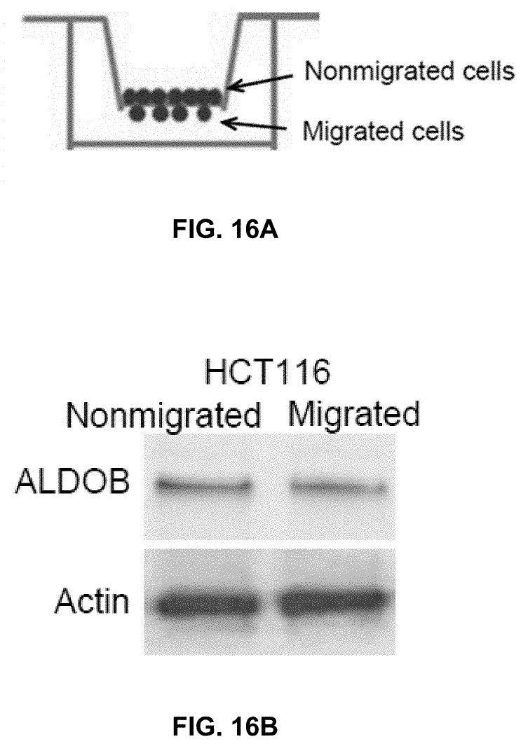

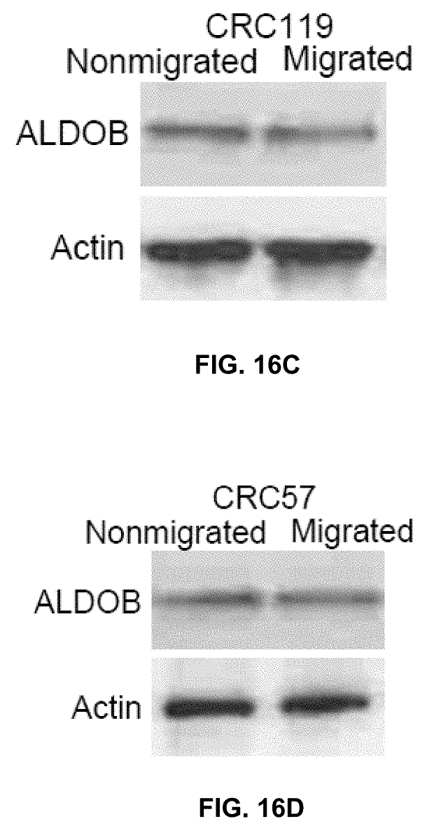

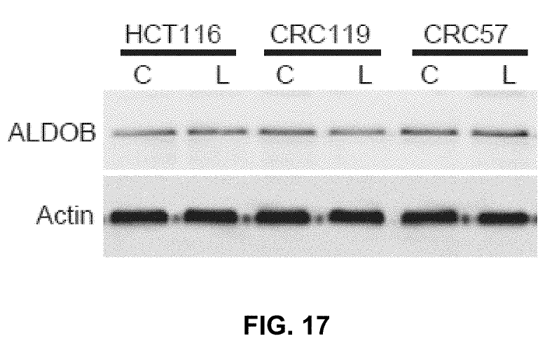

To investigate whether the liver environment can cause ALDOB upregulation in CRC cells, we injected HCT116, CRC119, and CRC57 cells directly into the mouse liver and cecum simultaneously. CRC tumors promptly formed in the livers and ceca, and we harvested the respective tumors 10 days after the injection, before metastases from cecum to liver could form, which takes 3-5 weeks in the cecum-injection model (FIG. 14). From the harvested tumors, CRC cells were isolated by FACS based on fluorescence. Western blot confirmed higher ALDOB levels in CRC cells isolated from the liver than from the cecum, while KHK and HK levels remained similar (FIGS. 15A-15C). On the other hand, migrated and non-migrated CRC cells in the transwell migration assay expressed similar ALDOB levels, suggesting that ALDOB is not associated with enhanced migration capability (FIGS. 16A-16D). Furthermore, after being cultured in vitro, disassociated tumor cells from liver and cecum express similar ALDOB levels (FIG. 17). Taken together, these data suggest that the liver environment can cause CRC cells to upregulate ALDOB.

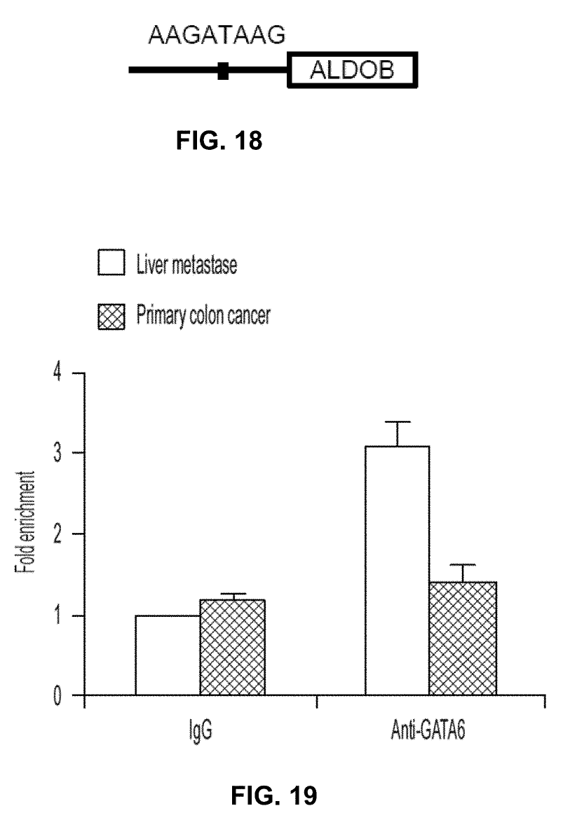

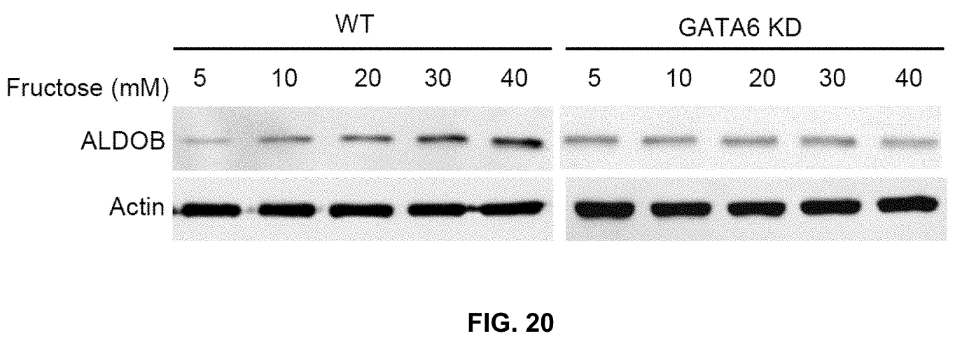

We analyzed 2 kb sequences of the ALDOB promoter and identified a putative GATA6 binding motif at −255 to −262 (FIG. 18). GATA6 expression has been reported to be significantly higher in CRC liver metastasis and correlates with poor prognosis and liver metastasis (Shen, F., et al., (2013) Oncol Rep, 30:1355-1361). ChIP-qPCR was then performed to validate this putative GATA6 binding motif, which showed that GATA6 binding to the ALDOB promoter was significantly enriched in CRC cells isolated from the liver than from the cecum (FIG. 19). We then cultured CRC cells in fructose-containing medium under hypoxia to mimic the liver environment. As shown by Western blot, ALDOB levels were up-regulated by fructose in a dose dependent manner, which was abrogated by GATA6 knockdown, indicating that ALDOB up-regulation in response to fructose is dependent on GATA6 (FIG. 20).

### Example 5: ALDOB Enhances Fructose Metabolism

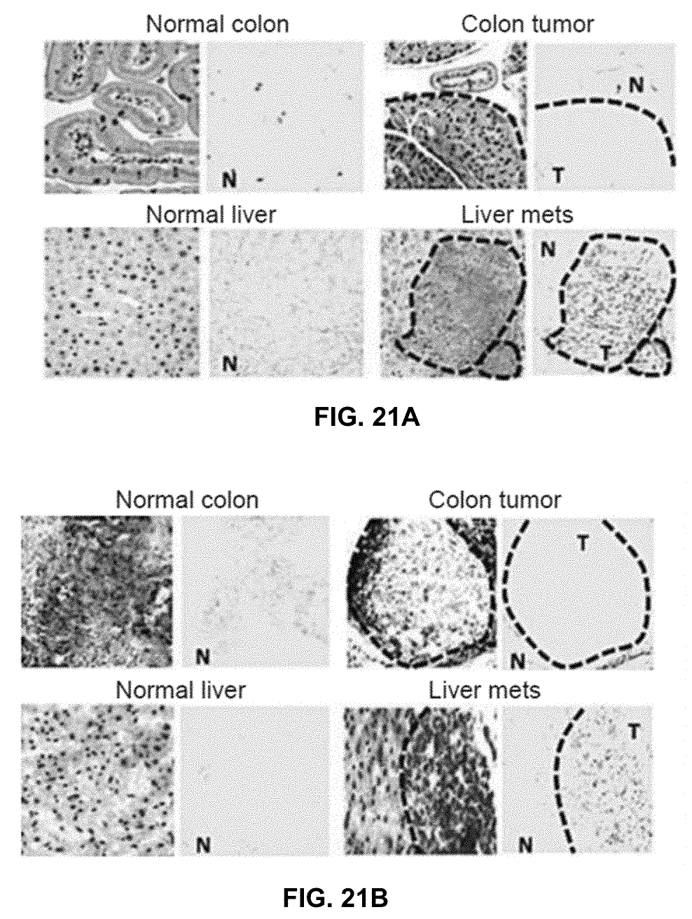

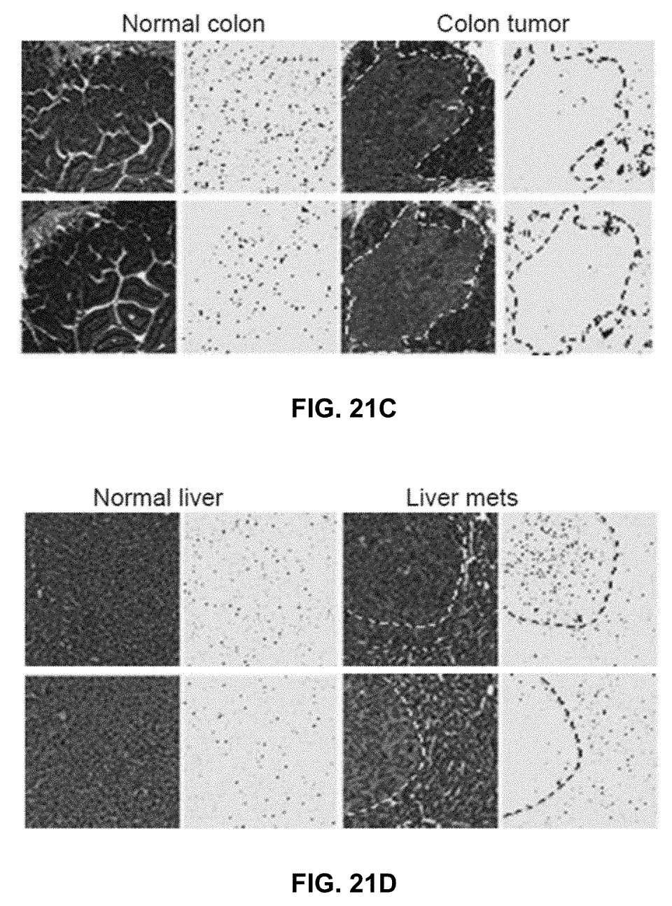

To determine if the products of ALDOB-mediated reaction contribute to glucose, glycogen, lactate, and lipid synthesis, all essential for sustaining highly proliferative cells, immunohistochemical staining was performed on tumor and normal tissues from primary and metastatic lesions to look for glycogen accumulation validated by periodic acid Schiff (PAS) with amylase digestion, and lipid deposits using Oil Red O (FIG. 21A-21D). Tumors growing in the liver were more productive in terms of glycogen and lipid synthesis.

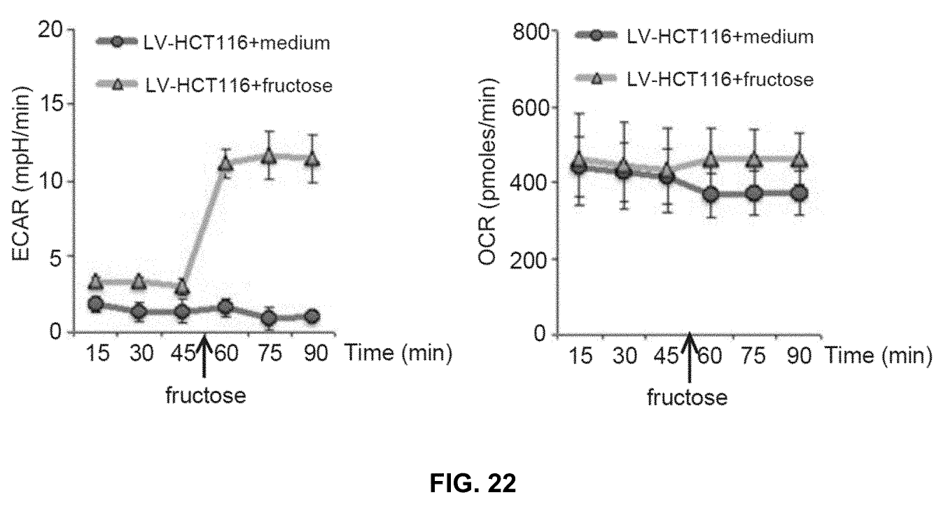

HCT116 cells were purified from the liver metastases based on mCherry expression (LVHCT116) and studied phenotypic changes in these cells by measuring cellular energetics parameters including extracellular acidification rate (ECAR, indicative of lactate production from glycolytic energy metabolism) and oxygen consumption rate (OCR, indicative of mitochondrial respiration) in the presence or absence of 11 mM fructose. The cells showed significant increase in ECAR and no change in OCR, which suggests that liver-derived CRC cells are capable of utilizing fructose to perform glycolytic functions (FIG. 22).

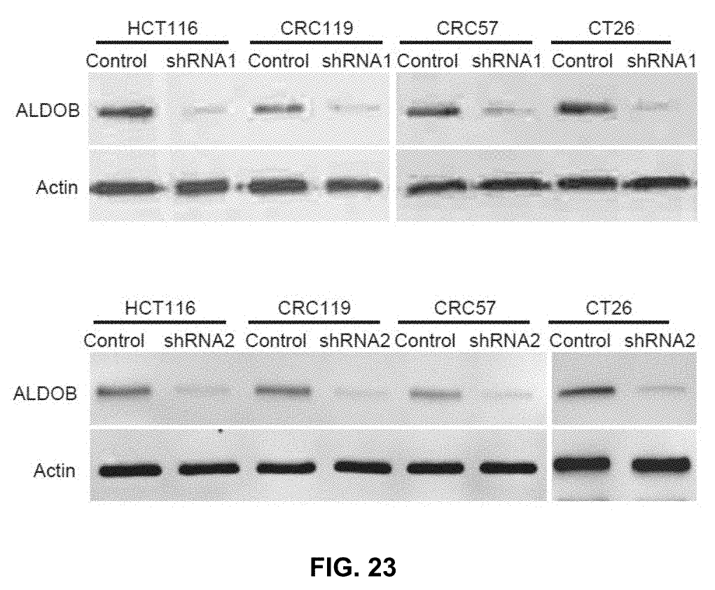

To assess the contribution of ALDOB to fructose metabolism, ALDOB was knocked down in CRC cells using two shRNAs and validated the knockdown efficiencies by Western blot (FIG. 23). ALDOB lentiviral shRNA constructs were purchased from Sigma Mission shRNA dataset. The lentiviral vectors were co-transfected with helper plasmids into 293T cells. The lentiviral vectors were transfected into 293T cells. The viral supernatant was collected 48 hours after transfection and was used to infect CRC cells.

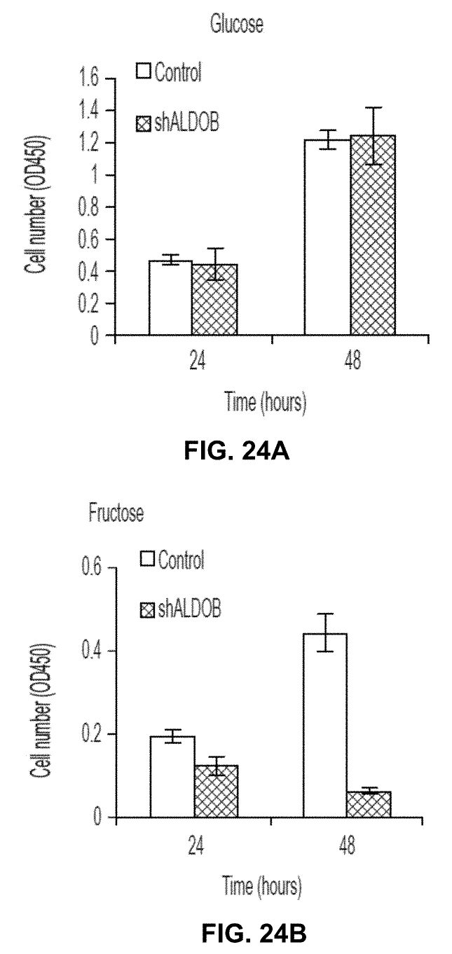

The control and ALDOB KD (ALDOB knockdown) cells grew equally well in glucose containing media with dialyzed FBS (FIG. 24A). However, ALDOB KD cells stopped growing in fructose-containing media with dialyzed FBS, while control cells grew normally and still doubled in 48 hours (FIG. 24B), suggesting that ALDOB plays an essential role in fructose metabolism for CRC cell growth.

Stable isotope tracing analysis was performed by adding [U-¹³C]fructose to culture medium with dialyzed FBS under hypoxia and tracing the labeled ¹³C in metabolites using Gas Chromatography Mass Spectrometry (GC-MS).

Isotope Tracing Analysis with ¹³C-Labeled Fructose

Cells were cultured in unlabeled fructose-containing medium for 24 hours, then switched into ¹³C labeled fructose-containing medium for 9 hours and 24 hours, respectively. To trace intracellular metabolite derivatization, metabolites were extracted using 80% cold methanol and dried under N2 gas-flow at 37° C. using an evaporator. Tert-butyldimethylsilyl (TBDMS) derivatized metabolites were performed as previously described (Ahn, W. S., et. al., (2011) Metab Eng, 13:598-609) with slight modifications. Briefly, metabolites were resuspended in 25 μL of methoxylamine hydrochloride (2% (w/v) in pyridine) and incubated at 40° C. for 90 minutes on a heating block. After brief centrifugation, 35 μL of MTBSTFA+1% TBDMS was added and the samples were incubated at 60° C. for 30 minutes. The derivatized samples were centrifuged for 5 minutes at 14,000 g and the supernatants were transferred to GC vials for GC-MS analysis. To measure ¹³C-enrichment of monomer sugars from acid hydrolysis of cell pellets, Cell pellet hydrolysis was performed in a two-step acid mediated process as previously described (McConnell, B. O., et. al., (2016) Anal Chem, 88:4624-4628). Labeling of monomer sugars was determined after aldonitrile propionate derivatization as previously described (Antoniewicz, M. R. et al., (2011) Anal Chem, 83:3211-3216). The derivatized samples were centrifuged for 5 minutes at 14,000 g and the supernatants were transferred to GC vials for GCMS analysis. GC-MS analysis was performed on an Agilent 7890B GC system equipped with a HP-5MS capillary column connected to an Agilent 5977A Mass Spectrometer. Mass isotopomer distributions were obtained by integration of ion chromatograms (Antoniewicz, M. R., et al., (2007) Anal Chem, 79:7554-7559) and corrected for natural isotope abundances (Fernandez, C. A., et al., (1996) J Mass Spectrom, 31:255-262).

Results

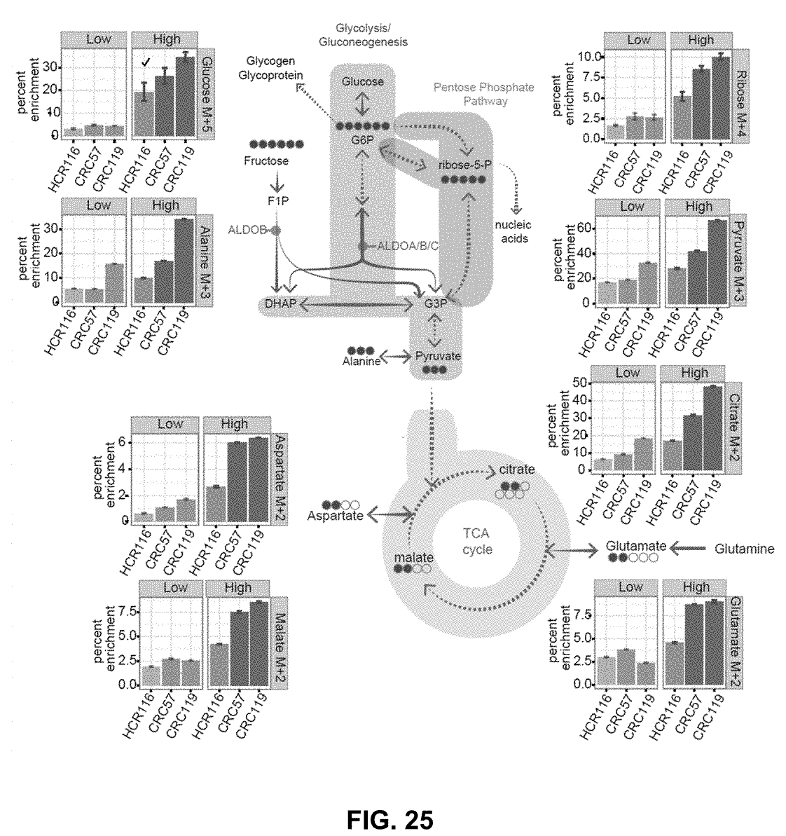

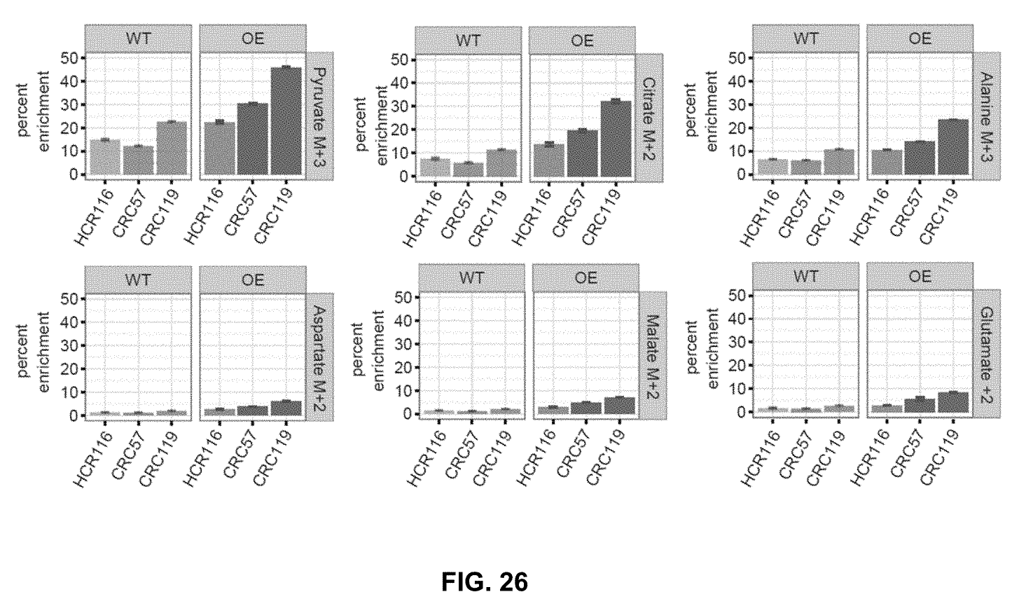

ALDOB KD cells had little [U-¹³C]fructose labeling, consistent with their quiescence in fructose-only dialyzed FBS media. Cells were compared with or without ectopic expression of ALDOB (ALDOB OE). Cecum-derived and liver-derived CRC cells in in vitro culture showed identical labeling results, so only tracing data from liver-derived CRC cells are presented below. As fructose enters glycolysis at the triose phosphate level, it contributes to lower glycolysis, as illustrated by enrichments in label incorporation of pyruvate, the terminal intermediate of glycolysis (FIG. 25 and FIG. 26). Label incorporation of Alanine, an amino acid closely downstream of pyruvate, is also enriched (FIG. 25 and FIG. 26). Label incorporation of M+2 citrate indicates that fructose contributes directly to acetyl CoA entry into the TCA cycle (FIG. 25 and FIG. 26). M+2 label incorporation decreases several folds from citrate to glutamate, and M+3 aspartate is <1% for all conditions tested, suggesting that pyruvate anaplerosis through carboxylation into TCA cycle is minimal and glutamine in the medium is likely the predominant anaplerotic carbon source (FIG. 25 and FIG. 26).

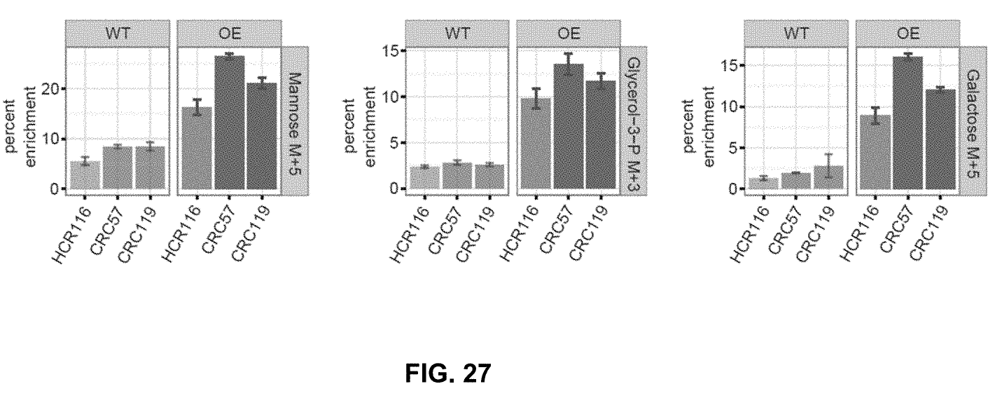

To assess whether upper glycolytic intermediates (e.g. G6P) and nucleotide precursors (e.g. ribose-5-phosphate) were labeled from [U-¹³C]fructose, cell pellet-derived glycogen and RNA were hydrolyzed into the monomer sugars (glucose and ribose, respectively) and measured the ¹³C-enrichment. Both glucose and ribose displayed enrichment from [U-¹³C]fructose (fragmentation in mass spectrometry results in loss of one carbon from the sugar, hence M+5 for glucose and M+4 for ribose, see methods (Long, C. P., et al., (2016) Metabl Eng, 37:102-113; McConnell, B. O., et al., (2016)) (FIG. 25). Other sugar monomers were also labeled (FIG. 27). Hence fructose is a source for upper glycolytic and the pentose phosphate pathway intermediates.

ALDOB expression greatly enhanced label incorporation of the above metabolites (FIG. 25 and FIG. 26), consistent with its important role in fructose metabolism. Overall, fructose, especially upon ALDOB expression, contributes to major pathways of central carbon metabolism (glycolysis/gluconeogenesis, PPP, and Pyruvate entry into TCA).

### Example 6: ALDOB Promotes CRC Liver Metastases

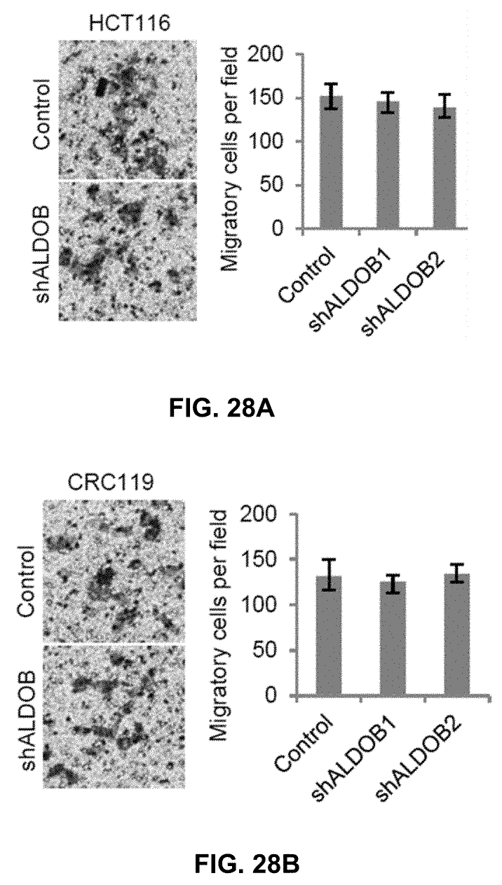

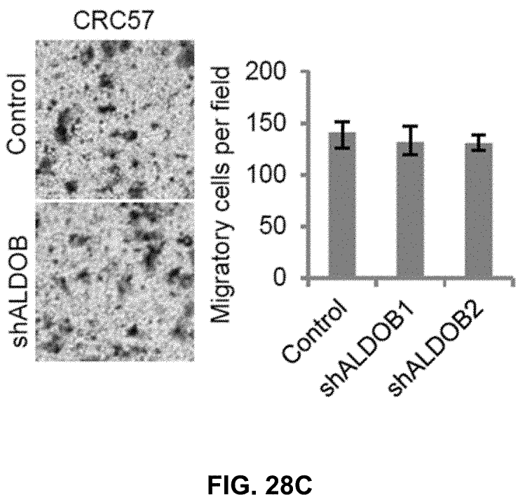

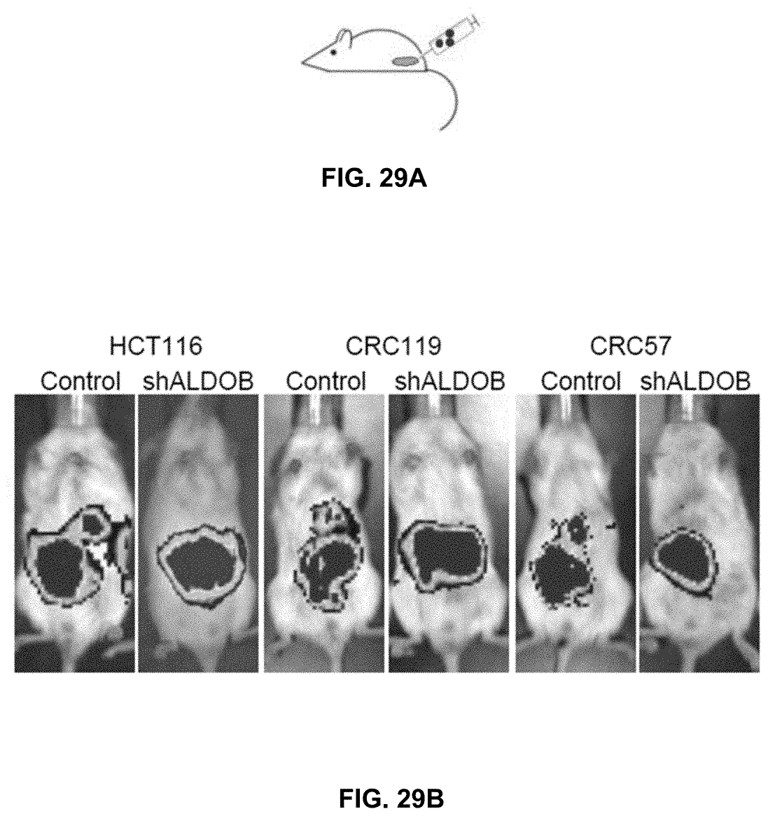

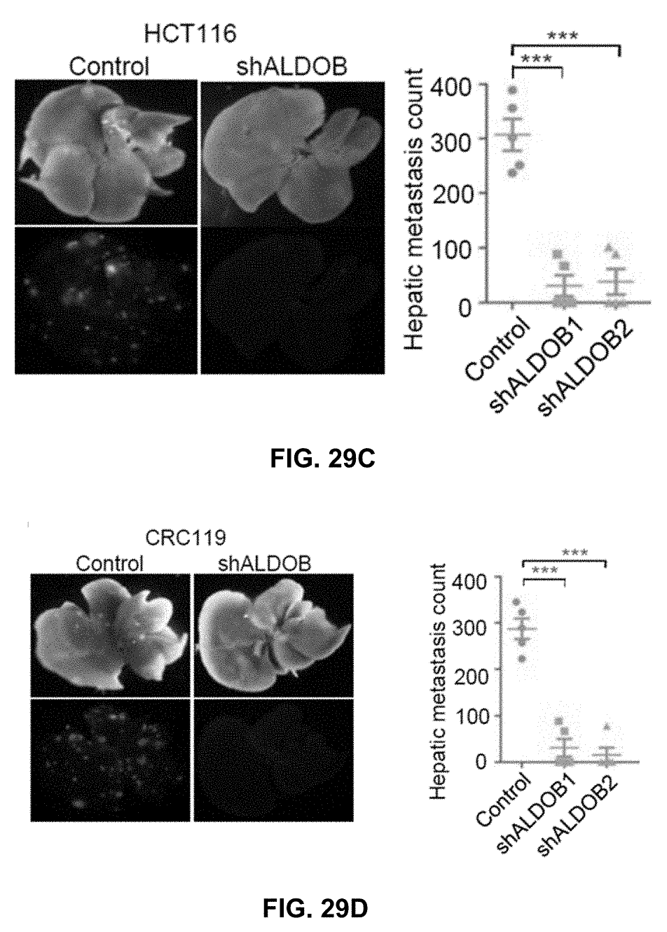

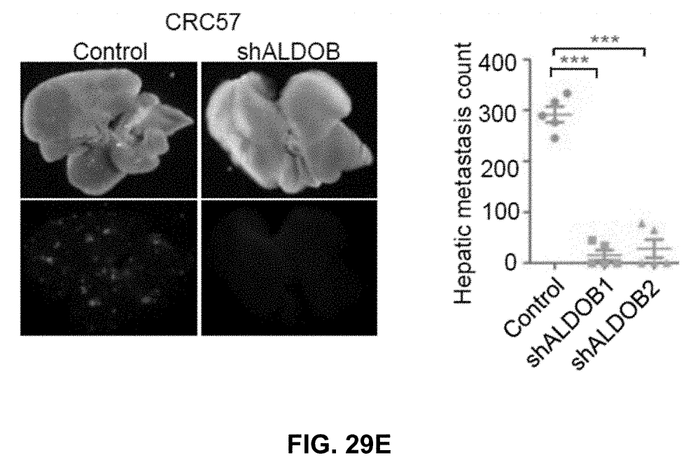

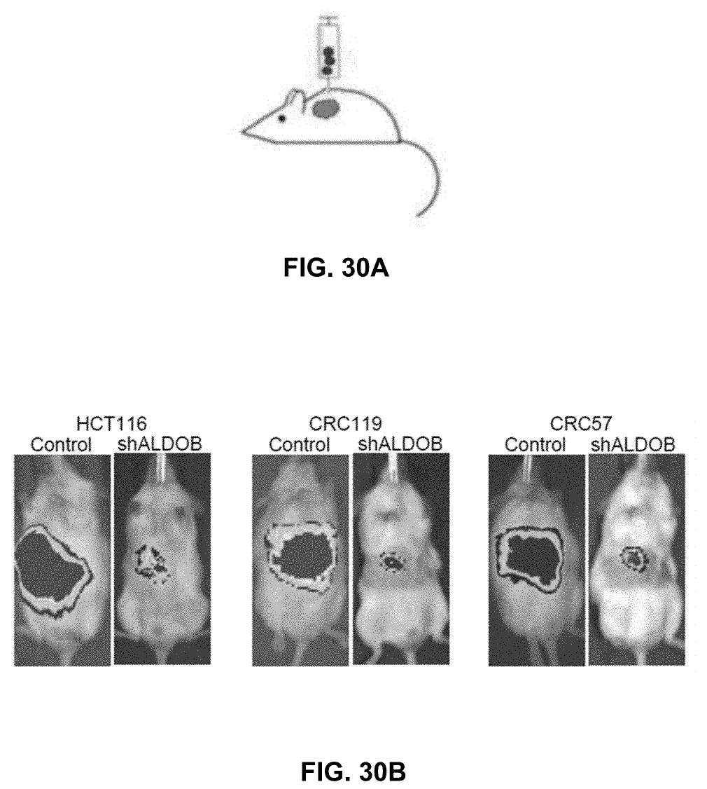

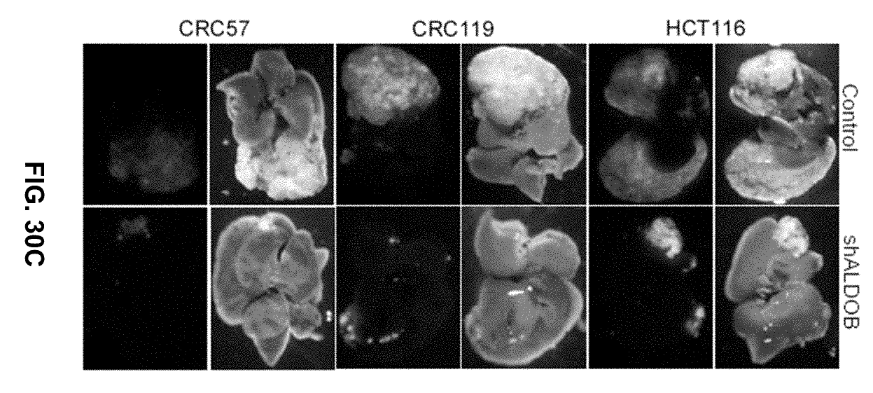

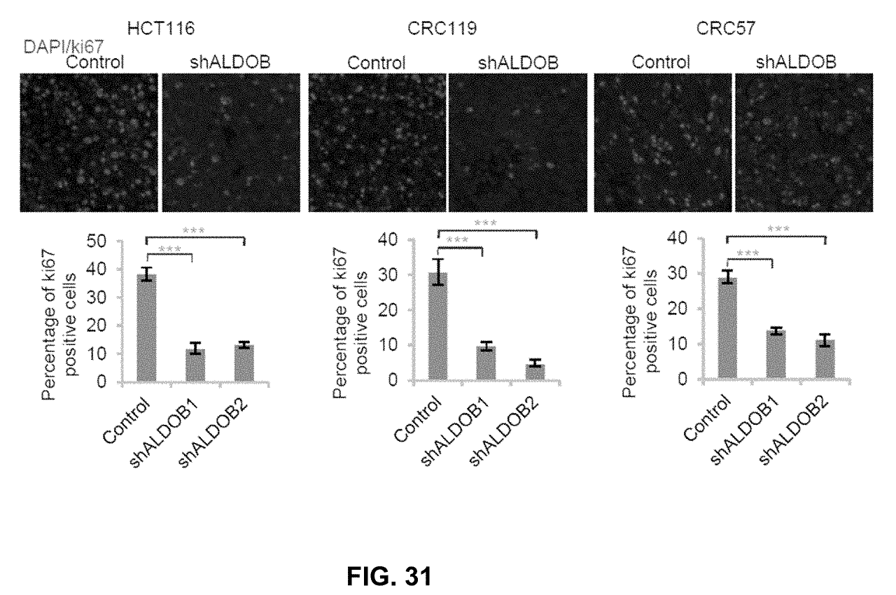

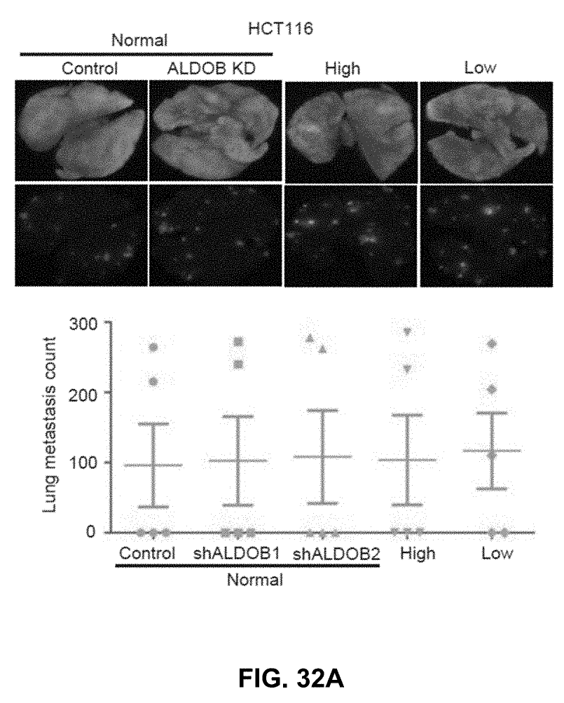

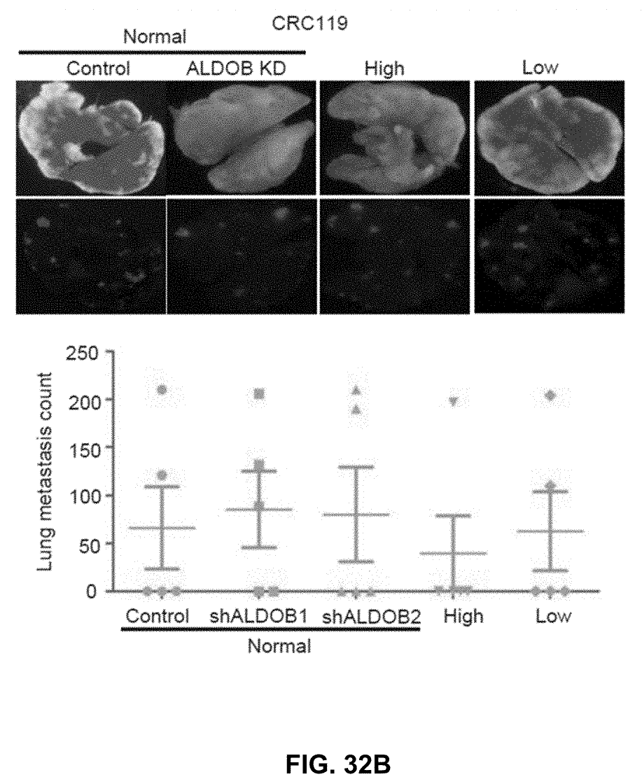

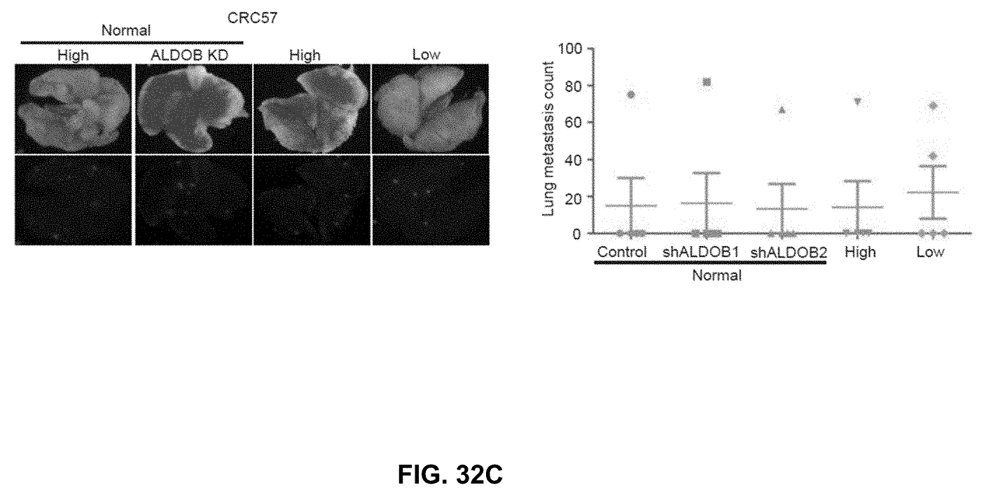

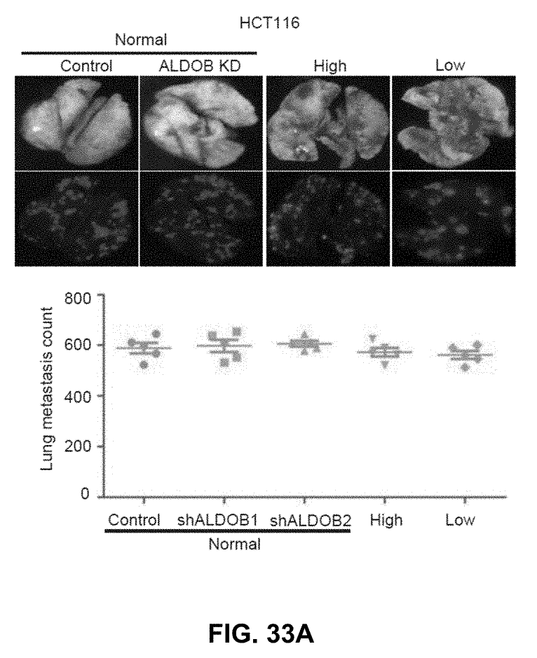

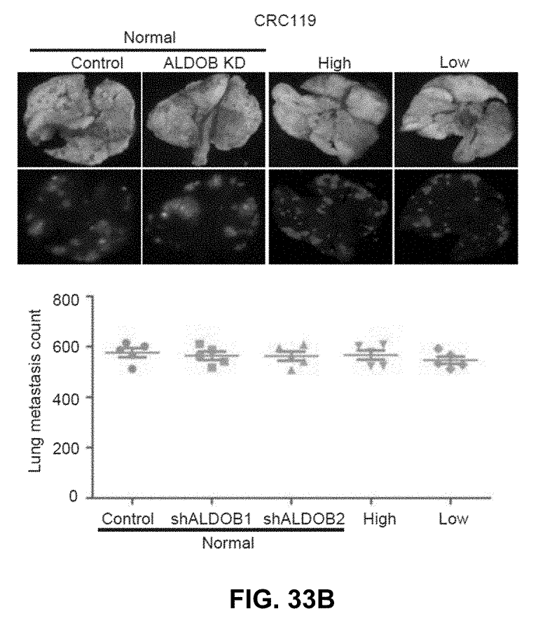

ALDOB knockdown in HCT116, CRC119 and CRC57 cells did not affect cell migration in vitro (FIGS. 28A-28C). However, while cecal-transplanted HCT116, CRC119, or CRC57 cells with control vectors developed liver metastases efficiently (5 out of 5 mice for all three cell lines), ALDOB knockdown suppressed CRC liver metastasis in the cecum injection model-injected HCT116, CRC119, or CRC57 cells with ALDOB knockdown by shRNA1 (SEQ ID NO: 1) developed detectable liver metastases in only 2, 2, and 2 out of 5 mice respectively and 2, 1, and 2 out of 5 mice by shRNA2 (SEQ ID NO:2) respectively (FIG. 29A-29E). Furthermore, the liver metastases grown from ALDOB knockdown cells were far fewer and much smaller than those grown from control cells. Intrahepatic injection was then performed to see whether ALDOB promotes CRC growth in the liver. HCT116, CRC119, and CRC57 cells with control vectors grew significantly bigger tumors than cells with ALDOB knockdown in the liver (FIGS. 30A-30C). Ki67 staining indicated that loss of ALDOB decreased proliferative rates of tumor cells in the liver (FIG. 31). ALDOB knockdown did not seem to affect CRC lung metastasis in the cecum injection model-control cells and ALDOB knockdown cells developed similar number of lung metastases (1-3 out of 5 mice); moreover, the sizes of the metastatic lung lesions were similar between the control group and the ALDOB knockdown group (FIG. 32A-32C). An alternative lung metastasis model via tail vein injection was then used of the control and ALDOB knockdown cells. All 5 mice injected with either control cells or ALDOB knockdown cells developed lung metastases of similar sizes (FIG. 33A-33B).

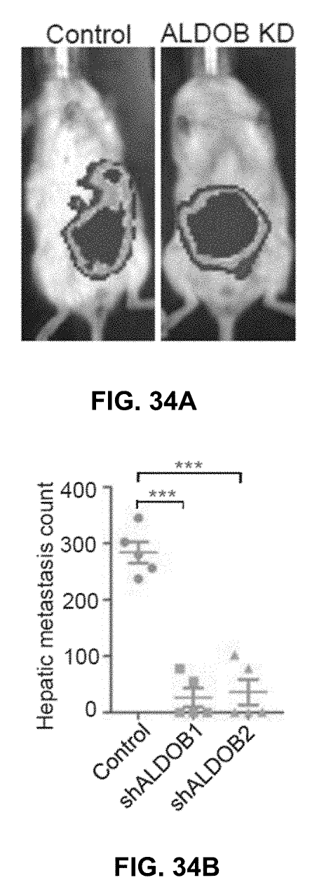

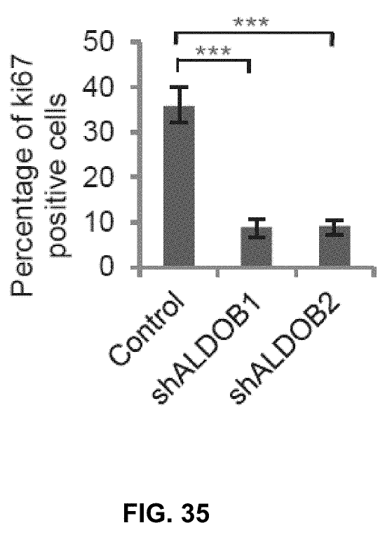

To take into consideration the effect of host immune system, ALDOB was knocked down in the mouse CRC cell line CT26, and injected them into immunocompetent BALB/c mice. Both cecum injection and intrahepatic injection models confirmed that loss of ALDOB suppressed CRC growth in the livers of immunocompetent mice compared to the control groups (5 mice per group) (FIG. 34A-34B). Ki67 staining indicated that loss of ALDOB decreased proliferation of CT26 cells in the liver (FIG. 35).

In sum, intrahepatic implantation indicates that the liver environment causes CRC cells to up-regulate ALDOB. Metabolomics and ¹³C-labeled fructose tracing studies indicate that ALDOB promotes fructose metabolism to fuel glycolysis, gluconeogenesis and the pentose phosphate pathway. ALDOB knockdown or dietary fructose restriction suppresses growth of CRC liver metastases, but not primary tumors or lung metastases, highlighting the importance of tumor environment.

### Example 7: Targeting Fructose Metabolism Suppresses Liver Metastases

It was next considered whether manipulating the level of fructose intake would impact tumor growth specifically in the liver.

Mice and Treatments

Select groups of mice were fed with a fructose-restricted and fructose-high diet purchased from Research Diets (New Brunswick, N.J.). Diet ingredients are available in Table 7.

TABLE 7
Ingredient of fructose-high, fructose-restricted and regular diet.

| | Diet | | |
|---|---|---|---|
| | Fructose-high | Fructose-restrict | Regular chow |
| Ingredient | diet | diet | diet |
| kcal/gm | 3.8 | 3.8 | 3.1 |
| Protein | 19% | 19% | 25% |
| Carbohydrate | 69% | 69% | 58% |
| Fat | 3% | 3% | 17% |
| Casein | 200 g | 200 g | |
| L-Cystine | 3 g | 3 g | |
| Corn Starch | 0 | 1590 g | |
| Maltodextrin | 0 | 528 g | |
| Sucrose | 0 | 0 | |
| Fructose | 720 g | 0 | |
| Cellulose, BW | 50 g | 50 g | |
| Soybean Oil | 30 g | 30 g | |
| t-Butylhydroquinone | 0.014 g | 0.014 g | |
| Mineral Mix | 35 g | 35 g | |
| S10022G | | | |
| Vitamin Mix | 10 g | 10 g | |
| V10037 | | | |
| Choline Bitartrate | 2.5 g | 2.5 g | |

Results

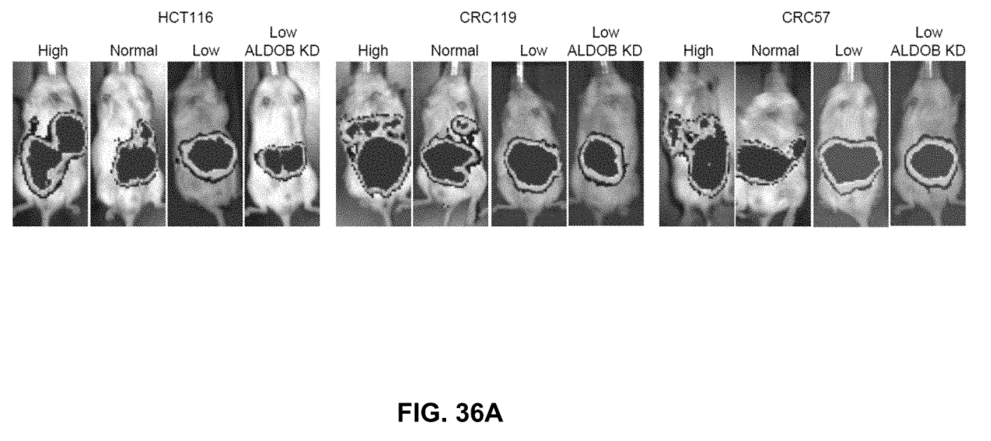

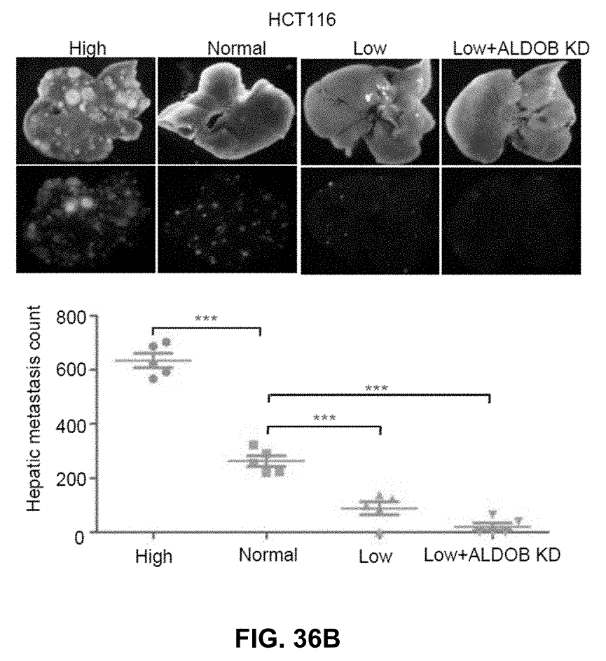

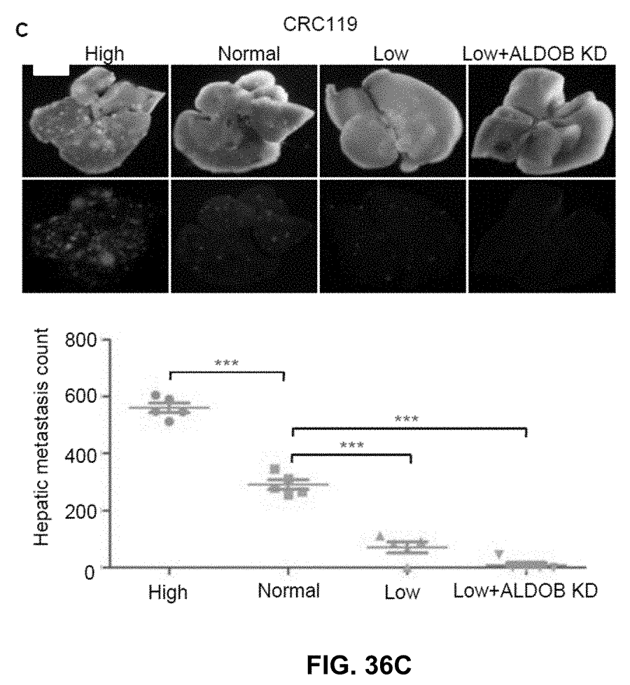

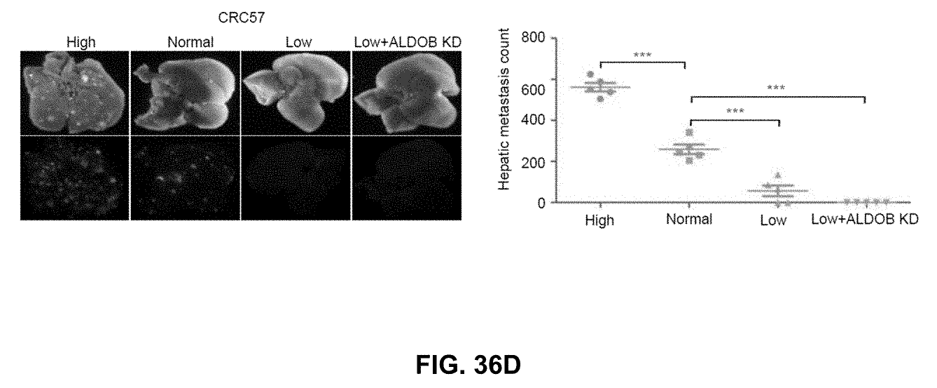

After cecum injection (5 mice per group), mice fed with a regulated diet with high fructose showed increased CRC liver metastases, while mice fed with a regulated diet devoid of fructose showed reduced liver metastases, compared to the control mice (FIGS. 36A-36D and Table 7).

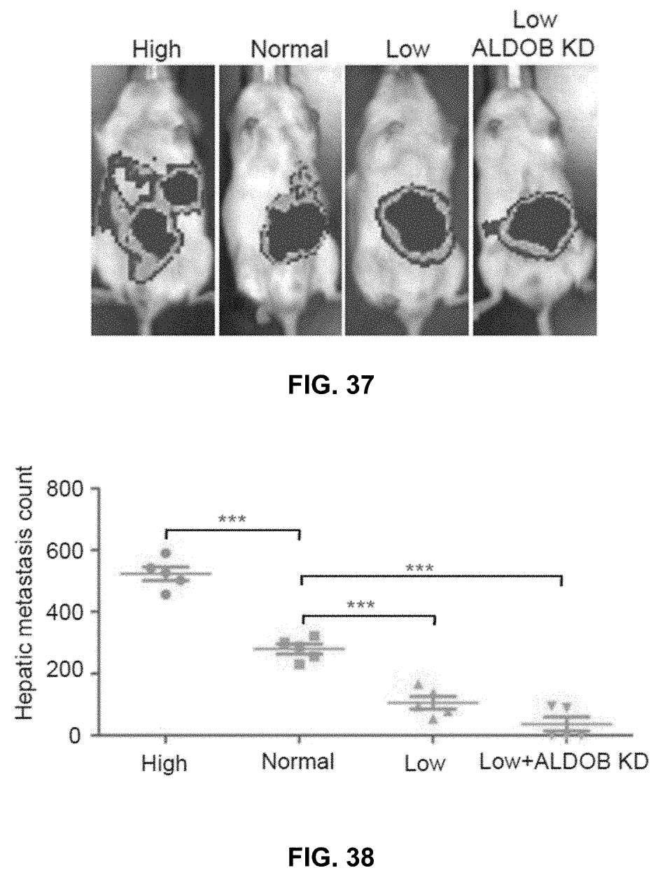

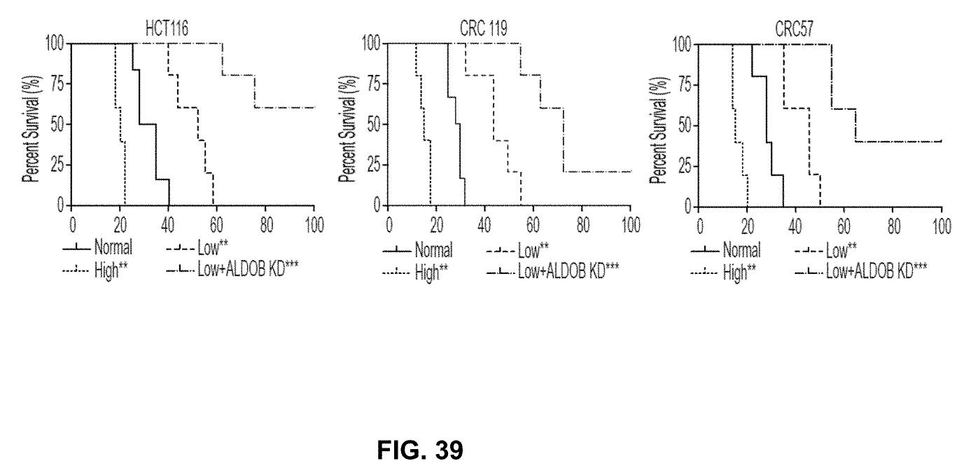

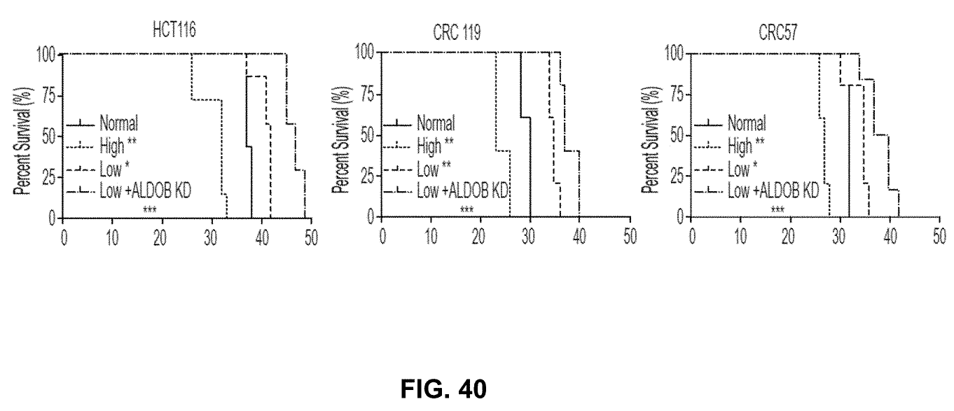

The two treatments were subsequently combined—mice were injected with ALDOB knockdown followed by a regulated diet devoid of fructose, which suppressed liver metastases as expected (FIGS. 36A-36D). Cecum injection of CT26 cells into immunocompetent BALB/c mice showed similar results with regard to the effect of fructose diets on liver metastases (FIG. 37 and FIG. 38). Consistently, high fructose diets reduced mouse survival, while low fructose diet and ALDOB knockdown prolonged mouse survival—the survival benefits were stronger in the intrahepatic injection model than in the cecum-injection model, presumably because the latter group was dying from their cecum tumors, which were largely unaffected by fructose diet or ALDOB knockdown (FIG. 39 and FIG. 40). Consistent with ALDOB knockdown, high fructose diet or fructose restriction did not affect lung metastasis in both cecum injection and tail vein injection model (FIGS. 32A-32C; FIGS. 33A-33B).

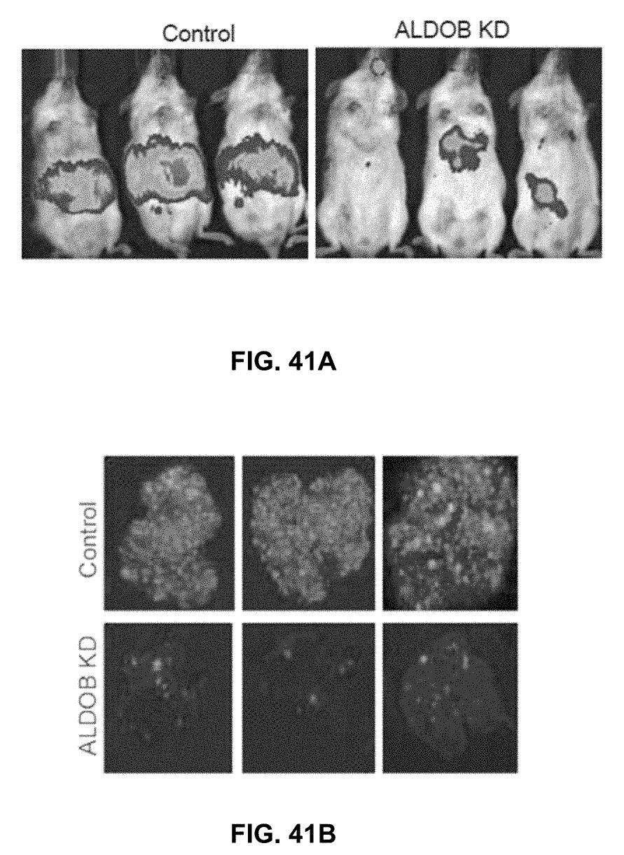

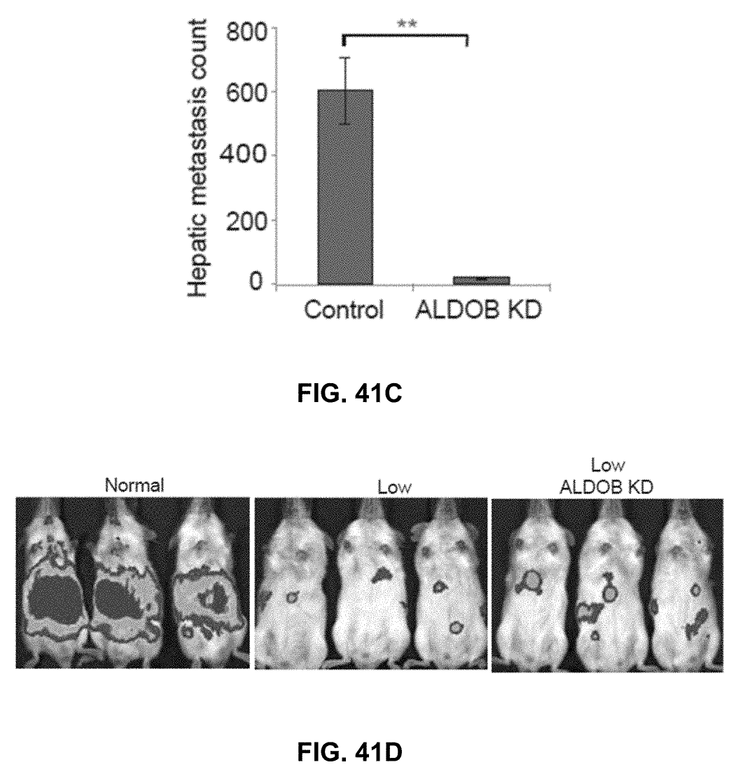

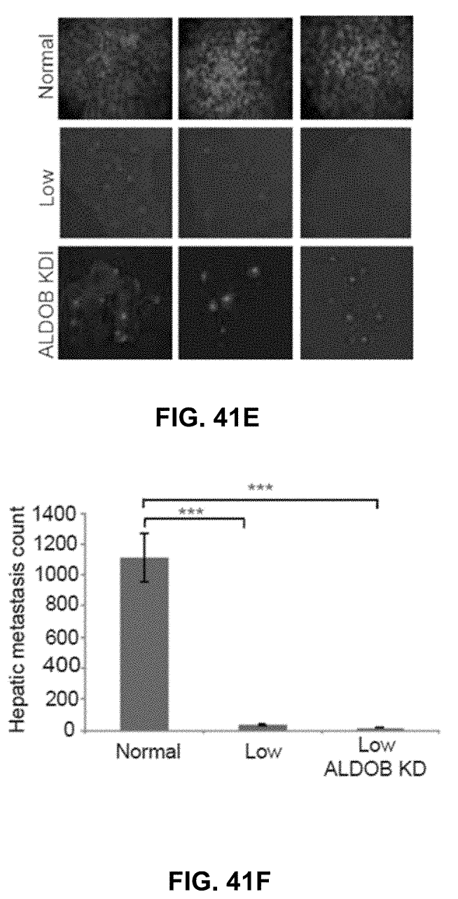

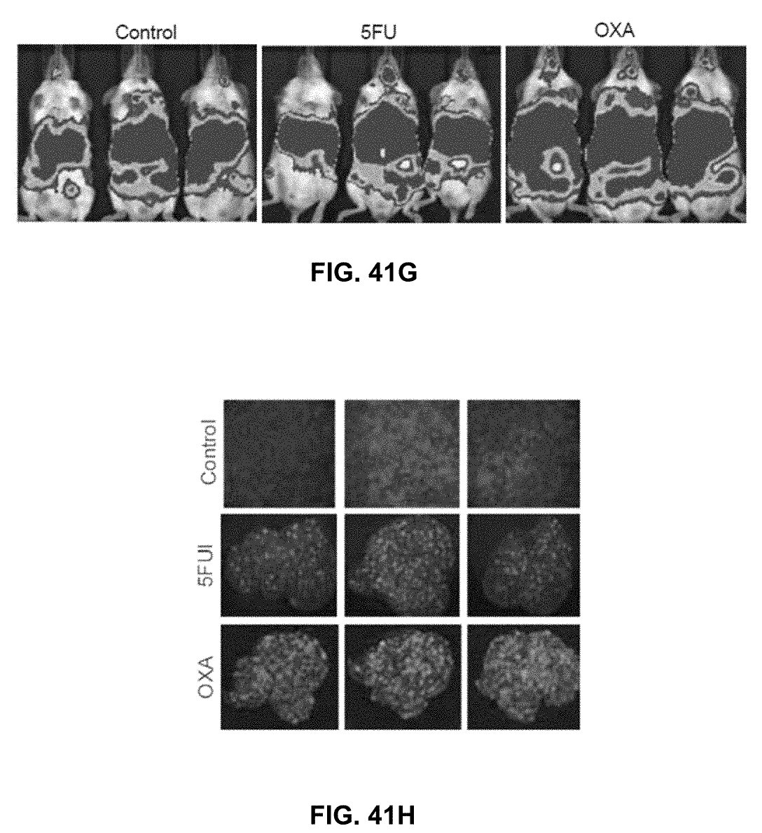

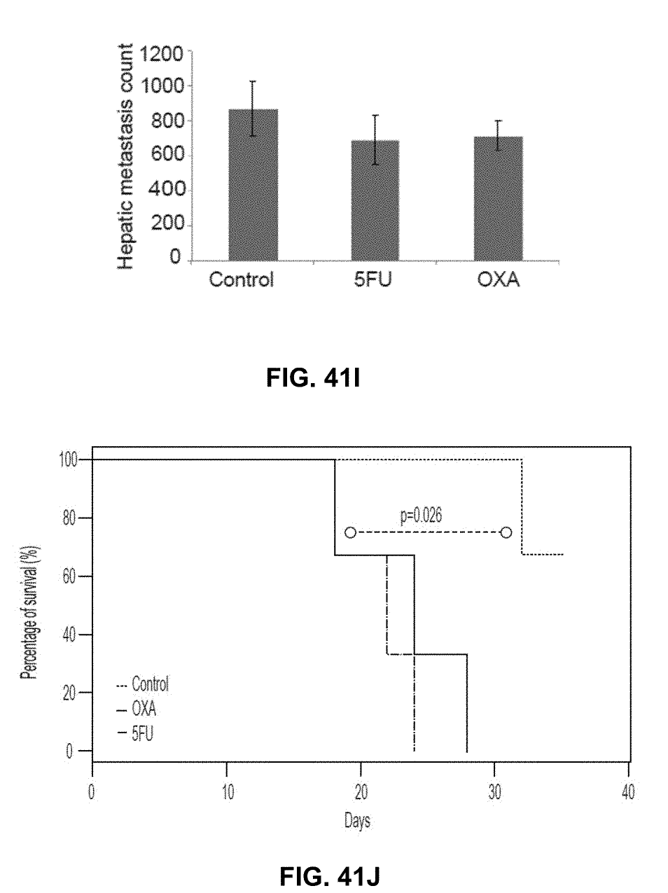

The role of ALDOB in promoting CRC tumor growth in the liver was validated using an alternative model. For this HCT116 lines (LV-HCT116) were generated that exhibited specific tropism to the liver through sequential passaging in the livers of NOD/SCID mice. ALDOB knockdown was carried out by transfecting LV-HCT116 cells with the same shRNA constructs. Consistent with the cecum injection model, ALDOB knockdown and fructose restriction suppressed CRC tumor in the liver (FIG. 41A-41F). With regard to suppression of LVHCT116 tumors in the liver, ALDOB knockdown and fructose restriction seem to be more effective than 5-Fluorouracil or Oxaliplatin, both of which are frontline chemotherapy for advanced and metastatic CRC (FIG. 41G-41J) (Alberts, S. R., et al., (2005) J Clin Oncology, 23:9243-9249; Andre, T., et al., (2004) New England J of Med, 350:2342-2351). Unlike ALDOB knockdown or fructose-restricted diet, 5-Fluorouracil or Oxaliplatin provided less benefit in terms of tumor suppression or survival. Hence, targeting ALDOB and fructose metabolism has the potential to impact the growth of liver metastases and be complementary to current chemotherapies.

Discussion

Current CRC chemotherapies do not distinguish the site of metastasis. As shown herein, metastatic CRC cells are capable of adjusting to nutrients change in their colonized organ. The diverse metabolites in the liver present a particularly intriguing case, given that colon epithelial cells probably depend more on alternative nutrients such as short chain fatty acids (SCFAs), e.g., butyrate. Fructose has been implicated in an array of metabolic diseases and positioned as a potentially harmful carbohydrate (Cantley, L. C. (2014) BMC Biology, 12:8-8). Fructose consumption has also been associated with clinical liver fibrosis (Abdelmalek, M. F., et al., (2012); Abdelmalek, M. F., et al., (2010)). Here, a role was identified of fructose in driving liver metastatic disease mediated by ALDOB. Inhibition of ALDOB or restriction of fructose can be effective in delaying metastatic onset or deterring metastatic growth.

Current CRC chemotherapies tend not to distinguish the organ environment of metastatic lesion. Our work suggests that metastatic cancer cells are capable of adjusting to nutrients change in their colonized organ. The diverse metabolites in the liver present a particularly intriguing case, given that liver is an important organ for cancer metastasis. Besides being the dominant site for CRC metastasis (70%) and seeding tertiary tumors in the lungs of CRC patients, liver is also a common metastatic site for breast, lung, kidney, esophagus, melanoma, ovary, uterus, pancreas, stomach cancer, and others. These studies demonstrate that the unique fructose-rich and hypoxic liver environment may contribute to its popularity for cancer metastasis, and liver metastasis of other cancer types may also up-regulate ALDOB in the liver. Fructose restriction and blocking ALDOB can be a viable strategy to suppress liver metastasis of other cancer types.

### Example 8: Ketohexokinase Silencing Suppresses Liver Metastases

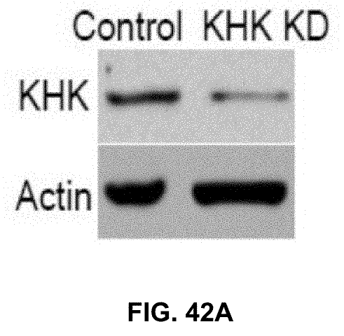

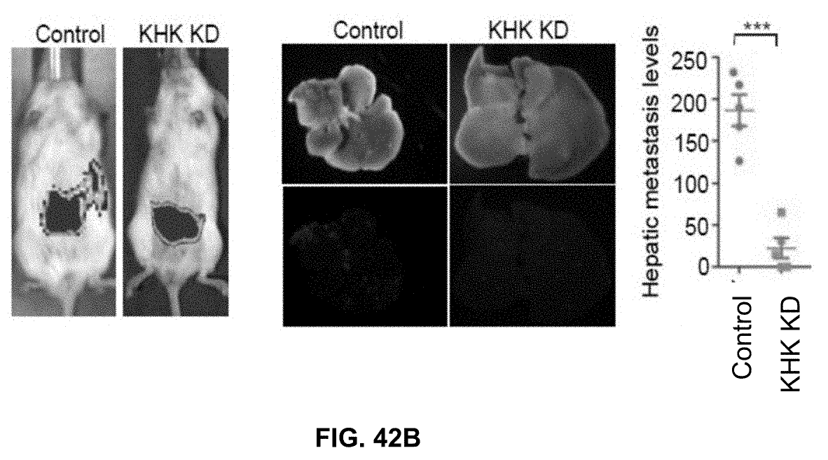

To determine if silencing ketohexokinase (KHK) can suppress liver metastases, KHK was knocked down using shRNA nucleic acid sequence SEQ ID NO:4 (FIG. 42A) in CRC cells and the cells were transplanted into the mouse cecum using the methods described in Example 6 with respect to ALDOB knockdown. As observed by IVIS luciferase in vivo images, bright field and fluorescent images of livers, and quantification of liver metastasis, KHK knockdown significantly suppressed metastatic CRC growth in the liver (FIG. 42B).

### Example 9: GLUT5 Silencing Suppresses Liver Metastases

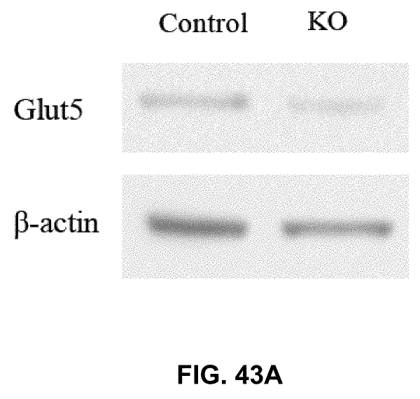

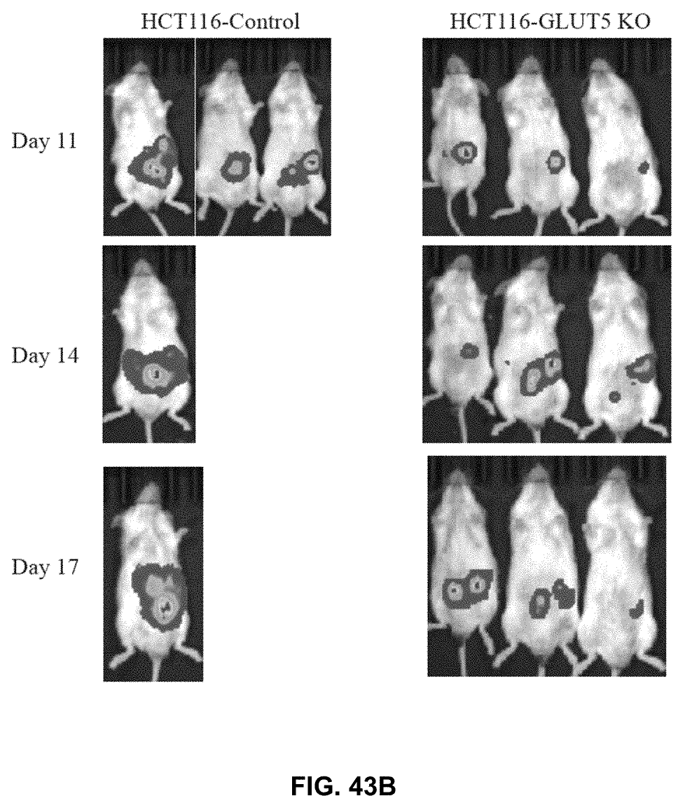

To determine if silencing GLUT5 can suppress liver metastases, GLUT5 was knocked down in HCT116 CRC cells and the cells were transplanted into the mouse cecum (FIG. 43A) using the methods described in Example 6 with respect to ALDOB knockdown. As observed by IVIS luciferase in vivo images of mouse livers on Day 11, Day 14, and Day 17 following the CRC transplant, GLUT5 knockdown suppressed metastatic CRC growth in the liver (FIG. 43B).

### Example 10: Inhibitors of Fructose Enzymes and Transporters Suppress Liver Metastases

To determine if various inhibitors of fructose enzymes and fructose transporters can suppress liver metastases, mice having metastatic liver cancer are administered the following inhibitors:

(1) KHK inhibitors (pyrimidinopyrimidine 1, indazole 2, pyrrolopyridine 7, pyrrolopyridine 9, pyrrolopyridine 10, pyridine 8, pyridine 11, and pyridine 12),

(2) aldolase inhibitors (phosphoric acid mono-(2,3-dioxo-butyl) ester),

(3) aldose reductase inhibitors (alrestatin, epairestat, fidarestat, imirestat, lidoestat, minalrestat, ponalrestat, ranirestat, salfredin B₁₁, sorbinil, tolrestat, zenarestat, and zopolrestat),

(4) sorbitol dehydrogenase inhibitors (CP-470711 (SDI-711) and WAY-135706),

(5) GLUT5 inhibitors (AGT-025, ab36057, ab41533, ab113931, ab87847, ab190555, ab111299, and OTI20E1), and,

(6) GLUT2 inhibitors (AGT-022, 600-401-GN3, LS-B15821, and LS-B4177).

Mice will first undergo cecum injection of CRC cells. A specific compound (e.g., pyridine 12) will then be administered to mice at week 4 following the cecum injection of the CRC cells when liver metastases are clearly detectable by IVIS in vivo imaging. In week 6, half of the mice in each group will be sacrificed, and their liver metastases will be imaged and scored compared to the control group. The other half of mice will continue receiving treatment with the inhibitor compound for survival studies (to generate Kaplan-Meyer survival curves). The protocol can be repeated using any of the inhibitors disclosed herein.

### Example 11: Inhibitors of Fructose Enzymes and Transporters Prevent or Reduce the Risk of Liver Metastases from a Primary Tumor

To determine if various inhibitors of fructose enzymes and fructose transporters can prevent or reduce the risk of liver metastases, mice having metastatic liver cancer are administered the following inhibitors:

(1) KHK inhibitors (pyrimidinopyrimidine 1, indazole 2, pyrrolopyridine 7, pyrrolopyridine 9, pyrrolopyridine 10, pyridine 8, pyridine 11, and pyridine 12),

(2) aldolase inhibitors (phosphoric acid mono-(2,3-dioxo-butyl) ester),

(3) aldose reductase inhibitors (alrestatin, epairestat, fidarestat, imirestat, lidoestat, minalrestat, ponalrestat, ranirestat, salfredin B₁₁, sorbinil, tolrestat, zenarestat, and zopolrestat),

(4) sorbitol dehydrogenase inhibitors (CP-470711 (SDI-711) and WAY-135706),

(5) GLUT5 inhibitors (AGT-025, ab36057, ab41533, ab113931, ab87847, ab190555, ab111299, and OTI20E1), and,

(6) GLUT2 inhibitors (AGT-022, 600-401-GN3, LS-B15821, and LS-B4177).

Mice will first undergo cecum injection of CRC cells. A specific compound (e.g., pyridine 12) will then be administered to mice at week 2 when the primary (cecal) tumors are detectable by IVIS in vivo imaging. In week 4 and week 6, the presence and growth of the metastatic tumors in the liver will be imaged and scored compared to the control group. In week 8, half of the mice in each group will be sacrificed, and their liver metastases will be imaged and scored. The other half of mice will continue receiving treatment for survival studies (to generate Kaplan-Meyer survival curves). The protocol can be repeated using any of the inhibitors disclosed herein.

Any patents or publications mentioned in this specification are indicative of the levels of those skilled in the art to which the disclosure pertains. These patents and publications are herein incorporated by reference to the same extent as if each individual publication was specifically and individually indicated to be incorporated by reference.

One skilled in the art will readily appreciate that the present disclosure is well adapted to carry out the objects and obtain the ends and advantages mentioned, as well as those inherent therein. The present examples along with the methods described herein are presently representative of preferred embodiments, are exemplary, and are not intended as limitations on the scope of the disclosure. Changes therein and other uses will occur to those skilled in the art which are encompassed within the spirit of the disclosure as defined by the scope of the claims.

We claim:

1. A method of treating cancer in a subject in need thereof, the method comprising administering to the subject an effective amount of a therapeutic agent capable of down-regulating and/or inhibiting a fructose enzyme or fructose transporter in a cell of the subject such that the cancer growth is suppressed.

2. The method of claim 1, wherein the cancer is a metastatic cancer.

3. The method of claim 1, wherein the cancer is a liver cancer.

4. The method of claim 1, wherein the cancer is a metastatic liver cancer.

5. The method of claim 1, wherein the therapeutic agent is an RNAi polynucleotide, a small molecule, or an antibody.

6. The method of claim 5, wherein the RNAi polynucleotide is selected from the group consisting of small interfering RNA (siRNA), short hairpin RNA (shRNA), and microRNA (miRNAs) oligonucleotides.

7. The method of claim 1, wherein the fructose enzyme or fructose transporter is selected from the group consisting of aldolase B (ALDOB), ketohexokinase (KHK), aldose reductase, sorbitol dehydrogenase, GLUT5, or GLUT2.

8. The method of claim 5, wherein the small molecule is an inhibitor of aldolase B (ALDOB), ketohexokinase (KHK), aldose reductase, sorbitol dehydrogenase, GLUT5, or GLUT2.

9. The method of claim 5, wherein the small molecule blocks de novo fructose synthesis in a cell of the subject.

10. The method of claim 5, wherein the small molecule is selected from the group consisting of pyrimidinopyrimidine 1, indazole 2, pyrrolopyridine 7, pyrrolopyridine 9, pyrrolopyridine 10, pyridine 8, pyridine 11, pyridine 12, any combinations thereof, and any salts, esters, isomers, and derivatives thereof.

11. The method of claim 10, wherein the small molecule is pyridine 12.

12. The method of claim 5, wherein the small molecule is selected from the group consisting of alrestatin, epairestat, fidarestat, imirestat, lidoestat, minalrestat, ponalrestat, ranirestat, salfredin B₁₁, sorbinil, tolrestat, zenarestat, zopolrestat, any combinations thereof, and any salts, esters, isomers, and derivatives thereof.

13. The method of claim 5, wherein the small molecule is CP-470711 (SDI-711) and any salts, esters, isomers, and derivatives thereof.

14. The method of claim 1, further comprising restricting the dietary intake of fructose in the subject.

15. The method of claim 14, wherein the subject has no dietary intake of fructose.

16. A method of treating cancer in a subject in need thereof, the method comprising administering to the subject an effective amount of a therapeutic agent capable of blocking de novo fructose synthesis in the subject such that the cancer growth is suppressed.

17. The method of claim 16, wherein the therapeutic agent is a small molecule inhibitor of or antibody against aldose reductase or sorbitol dehydrogenase.

18. A method of suppressing cancer growth in a subject in need thereof, the method comprising down-regulating and/or inhibiting a fructose enzyme in a cell of the subject.

19. The method of claim 18, wherein the fructose enzyme or fructose transporter is selected from aldolase B (ALDOB), aldose reductase, sorbitol dehydrogenase, ketohexokinase (KHK), GLUT5, or GLUT2.

20. The method of claim 18, wherein the cell is contacted with a fructose enzyme or fructose transporter inhibitor selected from the group consisting of pyrimidinopyrimidine 1, indazole 2, pyrrolopyridine 7, pyrrolopyridine 9, pyrrolopyridine 10, pyridine 8, pyridine 11, pyridine 12, alrestatin, epairestat, fidarestat, imirestat, lidoestat, minalrestat, ponalrestat, ranirestat, salfredin B₁₁, sorbinil, tolrestat, zenarestat, zopolrestat, CP-470711 (SDI-711), AGT-025, ab36057, ab41533, ab113931, ab87847, ab190555, ab111299, OTI20E1, AGT-022, 600-401-GN3, LS-B15821, LS-B4177, and any salts, esters, isomers, and derivatives thereof.


SEQUENCE LISTING (27 sequences)

SEQ ID NO: 1 - DNA, 58 bp, Artificial Sequence
  Synthetic oligonucleotide
ccggctcagaaattgcccagagcatctcgagatgctctgggcaatttctgagtttttg

SEQ ID NO: 2 - DNA, 58 bp, Artificial Sequence
  Synthetic oligonucleotide
ccgggtgggaatcaagttagaccaactcgagttggtctaacttgattcccactttttg

SEQ ID NO: 3 - DNA, 58 bp, Artificial Sequence
  Synthetic oligonucleotide
ccgggctacagactttgagaaggttctcgagaaccttctcaaagtctgtagctttttg

SEQ ID NO: 4 - DNA, 58 bp, Artificial Sequence
  Synthetic oligonucleotide
ccggcctaaggaggactcggagatactcgagtatctccgagtcctccttaggtttttg

SEQ ID NO: 5 - DNA, 58 bp, Artificial Sequence
  Synthetic oligonucleotide
ccgggcagcggatagacgcacacaactcgagttgtgtgcgtctatccgctgctttttg

SEQ ID NO: 6 - DNA, 59 bp, Artificial Sequence
  Synthetic oligonucleotide
ccgggactcggagataaggtgtttgctcgagcaaacaccttatctccgagtcttttttg

SEQ ID NO: 7 - DNA, 59 bp, Artificial Sequence
  Synthetic oligonucleotide
ccgggccagatgtgtctgctacagactcgagtctgtagcagacacatctggcttttttg

SEQ ID NO: 8 - DNA, 58 bp, Artificial Sequence
  Synthetic oligonucleotide
ccgggcactgctcatgcaacaatttctcgagaaattgttgcatgagcagtgctttttg

SEQ ID NO: 9 - DNA, 58 bp, Artificial Sequence
  Synthetic oligonucleotide
ccggcgccacatcatttgagcttatctcgagataagctcaaatgatgtggcgtttttg

SEQ ID NO: 10 - DNA, 58 bp, Artificial Sequence
  Synthetic oligonucleotide
ccggcccgtacagcttcattgtcttctcgagaagacaatgaagctgtacgggtttttg

SEQ ID NO: 11 - DNA, 58 bp, Artificial Sequence
  Synthetic oligonucleotide
ccggccttgctgttcaacaacatatctcgagatatgttgttgaacagcaaggtttttg

SEQ ID NO: 12 - DNA, 58 bp, Artificial Sequence
  Synthetic oligonucleotide
ccggcaagacgttcatagagatcaactcgagttgatctctatgaacgtcttgtttttg

SEQ ID NO: 13 - DNA, 58 bp, Artificial Sequence
  Synthetic oligonucleotide
ccgggcccacaatctcatactcaatctcgagattgagtatgagattgtgggctttttg

SEQ ID NO: 14 - DNA, 58 bp, Artificial Sequence
  Synthetic oligonucleotide
ccgggcaaacattctgtcattagttctcgagaactaatgacagaatgtttgctttttg

SEQ ID NO: 15 - DNA, 58 bp, Artificial Sequence
  Synthetic oligonucleotide
ccgggcacctcaacaggtaataatactcgagtattattacctgttgaggtgctttttg

SEQ ID NO: 16 - DNA, 58 bp, Artificial Sequence
  Synthetic oligonucleotide
ccggcgacgttctctctttctaattctcgagaattagaaagagagaacgtcgtttttg

SEQ ID NO: 17 - DNA, 58 bp, Artificial Sequence
  Synthetic oligonucleotide
ccgggctgaataagttctcttggatctcgagatccaagagaacttattcagctttttg

SEQ ID NO: 18 - DNA, 58 bp, Artificial Sequence
  Synthetic oligonucleotide
ccggtgtgcccatgtgtaccagaatctcgagattctggtacacatgggcacatttttg

SEQ ID NO: 19 - DNA, 58 bp, Artificial Sequence
  Synthetic oligonucleotide
ccggccattggatgagtcgggcaatctcgagattgcccgactcatccaatggtttttg

SEQ ID NO: 20 - DNA, 58 bp, Artificial Sequence
  Synthetic oligonucleotide
ccgggttcccagtgacaccaacattctcgagaatgttggtgtcactgggaactttttg

SEQ ID NO: 21 - DNA, 58 bp, Artificial Sequence; identical to SEQ ID NO: 19
  Synthetic oligonucleotide

SEQ ID NO: 22 - DNA, 58 bp, Artificial Sequence
  Synthetic oligonucleotide
ccggtgctgagaactttaaggtcttctcgagaagaccttaaagttctcagcatttttg

SEQ ID NO: 23 - DNA, 57 bp, Artificial Sequence
  Synthetic oligonucleotide
ccgggcgcctggagaactatcctatctcgagataggatagttctccaggcgcttttt

SEQ ID NO: 24 - DNA, 57 bp, Artificial Sequence
  Synthetic oligonucleotide
ccgggagaactatcctatccctgaactcgagttcagggataggatagttctcttttt

SEQ ID NO: 25 - DNA, 57 bp, Artificial Sequence
  Synthetic oligonucleotide
ccgggccgatacaatctgtcaccttctcgagaaggtgacagattgtatcggcttttt

SEQ ID NO: 26 - DNA, 57 bp, Artificial Sequence
  Synthetic oligonucleotide
ccgggccaatcgggatggtcactttctcgagaaagtgaccatcccgattggcttttt

SEQ ID NO: 27 - DNA, 57 bp, Artificial Sequence
  Synthetic oligonucleotide
ccggcgtccaagtctgtgaatgtaactcgagttacattcacagacttggacgttttt